(19) United States
(12) Patent Application Publication
(10) Pub. No.: US 2024/0310363 A1
(43) Pub. Date: Sep. 19, 2024

(54) METHODS FOR USING ENRICHED EXOSOMES AS A PLATFORM FOR MONITORING ORGAN STATUS

(71) Applicant: THE TRUSTEES OF THE UNIVERSITY OF PENNSYLVANIA, Philadelphia, PA (US)

(72) Inventor: Prashanth Vallabhajosyula, Bryun Mawr, PA (US)

(73) Assignee: THE TRUSTEES OF THE UNIVERSITY OF PENNSYLVANIA, Philadelphia, PA (US)

(21) Appl. No.: 18/625,699

(22) Filed: Apr. 3, 2024

Related U.S. Application Data
(62) Division of application No. 15/767,577, filed on Apr. 11, 2018.
(60) Provisional application No. 62/326,536, filed on Apr. 22, 2016.
(60) Provisional application No. 62/241,029, filed on Oct. 13, 2015.

(51) Int. Cl. G01N 33/50; C12Q 1/6883; G01N 33/68

(52) U.S. Cl. CPC G01N 33/5076; C12Q 1/6883; G01N 33/6893; C12Q 2600/106; C12Q 2600/158; C12Q 2600/178; G01N 2800/042; G01N 2800/245; G01N 2800/50; G01N 2800/52

(57) ABSTRACT

This present disclosure relates to the use of one or more biomarkers to monitor the conditional state of an organ or tissue, including transplanted tissue, or disease state, including diabetes, in a biological sample of a subject. Accordingly, this disclosure provides for: methods and kits for determining the presence of one or more biomarkers for organ or tissue rejection/injury or diabetes in a biological sample of a subject; methods for using the presence of such biomarkers to predict or diagnose organ or tissue rejection/injury or diabetes in a subject; and methods to select or modify a therapeutic regimen for a subject based on the use of such biomarkers.

### CROSS-REFERENCE TO RELATED APPLICATIONS

This application is a Divisional Application of U.S. patent application Ser. No. 15/767,577, filed Apr. 11, 2018, which is a U.S. National Stage Patent Application under 35 U.S.C. § 371 of International Application No. PCT/US2016/056752, filed on Oct. 13, 2016, which claims priority to U.S. Provisional Application Ser. No. 62/241,029, filed on Oct. 13, 2015 and U.S. Provisional Application Ser. No. 62/326,536, filed on Apr. 22, 2016, the contents of each of which are incorporated by reference herein in their entirety and to each of which priority is claimed.

### STATEMENT REGARDING FEDERALLY FUNDED RESEARCH

This invention was made with government support under 5R21AI111010-02 awarded by NIH/NIAID. The government has certain rights in this invention.

### SEQUENCE LISTINGS

The specification further incorporates by reference the Sequence Listing submitted herewith on Apr. 3, 2024. The Sequence Listing file, identified as 0814060564.xml, is 16,384 bytes and was created on Apr. 3, 2024. The Sequence Listing, electronically filed herewith, does not extend beyond the scope of the specification and thus does not contain new matter.

### BACKGROUND

Organ and tissue transplantation remains the only curative therapy in patients with end stage failure of the heart, lung, kidney, liver and pancreatic ß cells. Transplant organ rejection and immunosuppressive regimen related complications, however, remain the major causes of morbidity and mortality in transplant patients. With solid organ transplants such as kidney transplantation, serum markers such as creatinine are utilized to monitor rejection but their sensitivity and specificity remain low, e.g., creatinine sensitivity is still under 80% (Josephson M. A., 2011).

In islet transplant patients, once transplanted, the islets can produce insulin and actively regulate the level of glucose in the blood. If the cells are not from a genetically similar donor, however, the recipient may identify them as foreign, which may lead to an immune response and ultimately cause rejection of the transplanted tissue. As such, early detection of donor islet rejection is a clinical concern in the care of transplant recipients. Detection of rejection prior to the onset of graft dysfunction may allow for treatment of this condition with aggressive immunosuppression. Conversely, the ability to reduce immunosuppression in patients who are not experiencing donor islet rejection is desirable in order to minimize drug toxicity, infection, and malignancy. Existing biomarker platforms do not enable titration of immunosuppression. The current standard of clinical practices for transplant organ monitoring emphasize the critical need for development of more accurate, time-sensitive, non-invasive biomarker platforms in transplantation.

Exosomes are extracellular microvesicles (EV) released by many tissues into bodily fluids, including blood, urine, breast milk, and cerebrospinal fluid. Exosomes are tissue and major histocompatibility complex (MHC) specific, about 30-200 nm in diameter, and have in certain contexts been identified as carrying cargo reflecting the conditional state of the tissue releasing them. Exosomes also take part in the communication between cells by functioning as transport vehicles for proteins and nucleic acids. Since their discovery, a growing number of therapeutic applications are in development using exosomes derived from various producing cells, such as dendritic cells (DC), T lymphocytes, tumor cells and cell lines (Thery et al., 2002 and Delcayre et al., 2005).

### SUMMARY

The present disclosure provides methods for determining the presence of a biomarker indicative of organ or tissue rejection or tolerance in a subject undergoing transplantation. In certain embodiments, the detection of a change in one or more biomarkers of the present disclosure indicates the presence of donor organ or tissue rejection and/or injury in a subject. Thus, the methods of the present disclosure can also provide for early prognosis and diagnosis of organ or tissue rejection (e.g., identification of a biomarker prior to organ or tissue dysfunction).

In certain embodiments, the present disclosure provides methods for assessing the efficacy of a therapeutic or prophylactic therapy for preventing, inhibiting or treating organ or tissue rejection and/or injury in a subject, comprising determining the presence and/or level of a biomarker in a biological sample obtained from a subject prior to therapy and determining the presence and/or level of a biomarker in a biological sample obtained from the subject at one or more time points during therapeutic or prophylactic therapy, wherein the therapy is efficacious for preventing, inhibiting or treating organ or tissue rejection and/or injury in a subject when there is a change in the presence and/or level of the biomarker in the second or subsequent samples, relative to the first sample. In certain embodiments, the first sample can be obtained after therapeutic treatment has begun when there is no rejection or injury of the transplanted tissue.

In certain embodiments, the transplant or donor tissue can be pancreatic islet cells. In certain embodiments, the transplant or donor tissue can be beta islet cells. In certain embodiments, the transplant or donor tissue can be cultured beta islet cells.

In certain embodiments, the transplant or donor organ or tissue can be lung, pancreas, stomach, intestine, bladder, kidney, ovary, testis, skin, colorectal, breast, prostate, brain, esophagus, liver, placenta or fetus tissue. In certain embodiments, the transplant or donor organ can be kidney.

In certain embodiments, the present disclosure provides methods for predicting, diagnosing, or monitoring a disease in a subject, comprising determining the presence and/or level of a biomarker in a biological sample obtained from a subject. In certain embodiments, the present disclosure provides methods for predicting, diagnosing, or monitoring disease in a subject, comprising determining the presence and/or level of a biomarker in a biological sample obtained from a subject and comparing the presence and/or level to that obtained from a healthy subject. The methods of the present disclosure can also provide for early prognosis and diagnosis of a disease (e.g., identification of a biomarker prior to the onset of a disease).

In certain embodiments, the present disclosure provides methods for assessing the efficacy of a therapeutic or prophylactic therapy for preventing, inhibiting, or treating a disease in a subject, comprising determining the presence and/or level of a biomarker in a biological sample obtained from a subject prior to therapy and determining the presence and/or level of a biomarker in a biological sample obtained from the subject at one or more time points during therapeutic or prophylactic therapy, wherein the therapy is efficacious for preventing, inhibiting or treating the disease in a subject when there is a change in the presence and/or level of the biomarker in the second or subsequent samples, relative to the first sample. In some embodiments, the first sample is obtained after therapeutic treatment has begun.

In certain embodiments, the disease is a pancreatic disorder. In certain embodiments, the disease is pancreatitis. In certain embodiments the disease is a metabolic disease (e.g., morbid obesity related disorders, metabolic syndrome, etc.). In certain embodiments, the disease is diabetes. In certain embodiments, diabetes is type I diabetes, type II diabetes, or gestational diabetes.

In certain embodiments, the subject tissue can be pancreatic islet cells. In certain embodiments, the subject tissue can be beta islet cells.

In certain non-limiting embodiments, the biological sample can be a bodily fluid such as blood, urine, saliva, nasotracheal secretions, amniotic fluid, breast milk or ascites. In certain embodiments, the biological sample can be a blood sample. In specific non-limiting embodiments, one or more biomarkers can be detected in one or more biological samples from a subject.

In certain embodiments, the biomarker is a pool of one or more organ- or tissue-derived microvesicles (from the donor and/or subject), e.g., exosomes, wherein a change in a physical characteristic, e.g., the size and/or number, and/or the profile of the microvesicles is prognostic of and/or indicative of donor organ or tissue rejection and/or injury or diabetes in a subject.

In certain embodiments, the biomarker is the change in the number of donor organ specific exosomes. In certain embodiments, the biomarker is a decrease in the number of donor organ specific exosomes. In certain embodiments, the donor organ specific exosomes are donor islet specific exosomes. In certain embodiments, the donor organ specific exosomes are donor kidney specific exosomes.

In certain embodiments, the decrease in the number of donor specific exosomes occurs prior to the reoccurrence of hyperglycemia and/or diabetes. In certain embodiments, the decrease in the number of donor specific exosomes occurs prior to the reoccurrence of hyperglycemia and/or diabetes without the occurrence of beta islet cell rejection. In certain embodiments, the decrease in the number of donor specific exosomes can provide an early prognosis and/or diagnosis of hyperglycemia and/or diabetes. In certain embodiments, the decrease in the number of donor specific exosomes can provide an early prognosis and/or diagnosis of hyperglycemia and/or diabetes without the occurrence of beta islet cell rejection. In certain embodiments, the early prognosis and/or diagnosis can result in the treatment of the patient to prevent the immune response before the induction of hyperglycemia and/or diabetes.

In certain embodiments, the biomarker is a protein isolated from a pool of one or more organ- or tissue-derived microvesicles (from the donor and/or subject), e.g., exosomes, and the presence of the protein is detected, e.g., using a reagent which directly or indirectly binds the protein. In certain embodiments, the reagent can be an antibody, an antibody derivative, an antigen-binding antibody fragment and a non-antibody peptide which specifically binds the protein. In certain embodiments, the antibody or antigen-binding antibody fragment is a monoclonal antibody or antigen-binding fragment thereof, or a polyclonal antibody or antigen-binding fragment thereof. In certain embodiments, the protein biomarker can be detected by biophysical techniques such as mass spectrometry.

In certain embodiments, the protein isolated from a pool of one or more organ- or tissue-derived microvesicles (from the donor and/or subject) can be heat shock cognate protein 71 (Hsc-70), angiopoietin-1, hemopexin, and/or complement C3. In certain embodiments, the change in the expression levels of heat shock cognate protein 71 (Hsc-70), angiopoietin-1, hemopexin, and/or complement C3 in the pool of one or more organ- or tissue-derived microvesicles can be a biomarker for the rejection of the donor organ or tissue. In certain embodiments, the change in expression of at least one, at least two, at least three, or at least four of heat shock cognate protein 71 (Hsc-70), angiopoietin-1, hemopexin, and complement C3 in the pool of one or more organ- or tissue-derived microvesicles can be a biomarker for the rejection of the donor organ or tissue. In certain embodiments, the change in expression of heat shock cognate protein 71 (Hsc-70) is a decrease in expression as compared to a control (e.g., before rejection or normal patient). In certain embodiments, the change in expression of angiopoietin-1 is a decrease in expression as compared to a control (e.g., before rejection or normal patient). In certain embodiments, the change in expression of hemopexin is an increase in expression as compared to a control (e.g., before rejection or normal patient). In certain embodiments, the change in expression of complement C3 is an increase in expression as compared to a control (e.g., before rejection or normal patient).

In certain embodiments, the biomarker can also be a transcribed polynucleotide or portion thereof, e.g., a mRNA, miRNA snRNA, piRNA, lncRNA (long non-coding), or a cDNA, isolated from a pool of one or more organ- or tissue-derived microvesicles (from the donor and/or subject), e.g., exosomes. In certain embodiments, detecting a transcribed polynucleotide includes amplifying the transcribed polynucleotide. In certain non-limiting embodiment, the nucleic acid biomarker can be detected by RT-PCR and/or microarray analysis.

The disclosure also provides kits for diagnosing or assessing the conditional state of the donor organ or tissue, for monitoring the condition of a donor organ or tissue of a subject and for assessing the efficacy of a therapeutic treatment regime of a subject, where the kit containing means or reagents useful for detecting the biomarkers in a biological sample. In certain embodiments, the donor tissue can be pancreatic islet cells. In certain embodiments, the donor tissue can be islet beta cells. In certain embodiments, the donor tissue can be cultured beta islet cells. In certain embodiments, the tissue can be the subject's native pancreatic islet beta cells.

The disclosure also provides kits for predicting, diagnosing or monitoring diabetes in a subject and for assessing the efficacy of a therapeutic treatment regime of a subject, where the kit containing means or reagents useful for detecting the biomarkers in a biological sample.

### BRIEF DESCRIPTION OF THE DRAWINGS

FIG. 1. The xenoislet transplantation mouse model. Athymic mice (Nu/J strain) were made diabetic with streptozotocin treatment followed by transplantation with cultured human islets to correct the diabetic state.

FIGS. 2A-2D. A) Western blot analysis of exosomes isolated from supernatants of in vitro cultures of human pancreatic islets and naive, athymic mouse (recipient animal) plasma exosomes for human MHC class I molecules, HLA-A, HLA-B, and HLA-C is shown, along with β-actin control. B) NanoSight fluorescence images of exosomes from human pancreatic islet culture are shown for human MHC molecules, HLA-A, HLA-B, and HLA-C. IgG isotype control is also shown. C) Representative blood glucose curves for six diabetic recipient animals after islet transplantation showed normoglycemia. D) Extracellular microvesicles were analyzed on Western blot for exosome markers CD63 and flotillin-1 and apoptotic body marker cytochrome c.

FIG. 3. Recipient plasma total exosome pool analyzed on NanoSight nanoparticle detector on light scatter and fluorescence modes for donor human islet specific MHC signal using anti-HLA-A quantum dot.

FIGS. 4A-4C. A) Recipient plasma total exosome pool analyzed on NanoSight nanoparticle detector on light scatter and fluorescence modes for donor human islet specific MHC signal using anti-HLA-C quantum dot. B) Western blot analysis of total plasma exosomes showed HLA-A signal in xenoislet sample, but not the naive mouse sample. Positive controls included exosomes from human islet culture supernatant and human plasma. C) NanoSight analysis of recipient plasma exosomes from day 14 after islet graftectomy for human MHC signal using anti-HLA-A quantum dot were negative for TISE (p<0.01). Representative image from 1 out of 6 animals shown.

FIG. 5. Representative histology of the transplanted islet mass is shown from N-xeno animal. Hematoxylin and cosin histology staining of transplanted human islet mass under the mouse renal capsule showed islet clusters without leukocytic infiltration. Immunohistochemistry for insulin (red) and glucagon (green) confirmed presence of viable islet cells.

FIG. 6. Schematic of the method of enriching transplant islet specific exosomes (TISE) from recipient mouse plasma.

FIG. 7. Transmission electron microscopy of anti-HLA-A bound fraction of exosomes. Nanovesicles (arrow) primarily in the 40 nm to 100 nm range were seen.

FIG. 8. NanoSight light scatter and fluorescence analysis of HLA-A unbound exosome (EV) fractions from naive mouse and xenoislet plasma samples tested for HLA-A positive signal.

FIG. 9. Western blot analysis of HLA-A bound and unbound EV fractions assessed for the expression of HLA-A and HLA-B showed that HLA-A bound exosomes in xenoislet recipient plasma were enriched for the human specific MHC signal.

FIG. 10. NanoSight light scatter and fluorescence analysis of HLA-A bound and unbound EV fractions from xenoislet plasma samples tested for MHC class I and IgG isotype signal.

FIG. 11. NanoSight light scatter and fluorescence analysis of HLA-A bound exosome fraction tested for human specific leukocytes markers using quantum dots conjugated to anti-human CD3 (T cell), anti-human CD19 (B cell), and anti-human CD14 (monocyte). HLA-A bound exosomes were negative for human leukocyte markers in xenoislet samples.

FIG. 12. NanoSight light scatter and fluorescence analysis of HLA-A bound versus unbound EV fractions for: normoglycemic xenoislet plasma, naive human plasma, naive mouse plasma, human islet culture supernatant. For each sample, HLA-A unbound fraction was analyzed for HLA-A absence using anti-HLA-A quantum dot to make sure that there was optimal capture of HLA-A expressing exosomes.

FIG. 13. Western blot analysis of HLA-A bound EV fractions examining FXYD2 expression show that only xenoislet samples express beta cell specific markers.

FIG. 14. NanoSight and Western blot analysis of HLA-A bound plasma EV fraction 21 days after islet graftectomy tested for FXYD2 signal and expression, respectively, shows loss of beta cell markers with islet graft removal.

FIG. 15. Western blot analysis of HLA-A unbound EV fractions analyzed for the presence of islet endocrine hormones insulin, glucagon, and somatostatin. HLA-A unbound exosomes did not show the presence of islet specific hormone markers. Protein from islet graftectomy lysate (labeled as Islet graft) served as positive tissue control.

FIG. 16. Western blot analysis of HLA-A bound EV fractions analyzed for the presence of islet endocrine hormones insulin, glucagon, and somatostatin. Protein from islet graftectomy lysate (labeled as Islet graft) served as positive tissue control.

FIG. 17. Western blot analysis of HLA-A bound EV fraction from islet graftectomy samples analyzed for the presence of insulin. Protein from islet graftectomy lysate (labeled as Islet graft) served as positive tissue control.

FIG. 18. RT-PCR analysis of insulin, glucagon, and somatostatin expression in the HLA-A bound EV fractions. RNA extracted from islet graftectomy tissue (labeled as Islet graft) served as positive control. This showed that HLA-A bound exosomes in xenoislet samples also express islet endocrine hormone specific mRNA.

FIG. 19. RT-PCR analysis of FXYD2_{γa} expression in the HLA-A bound EV fractions. RNA extracted from islet graftectomy tissue (labeled as Islet graft) served as positive control.

FIG. 20. Proteomic and RNA profiling of TISE in the xenoislet model. TISE were analyzed via mass spectrometry for profiling of its protein cargo. Results of two independent experiments from N-xeno animals receiving human islets from different donors.

FIG. 21. Electrophoresis gel of total RNA cargo and bioanalyzer size analysis showing enrichment of small RNA in the transplant islet specific exosomes compared to the islet graft tissue RNA cargo. Levels are shown as fold expression over the median value for the microarray set.

FIG. 22. Long RNA data showing differential expression of islet endocrine hormone mRNAs in transplant islet specific exosomes compared to the islet tissue. FXYD2 mRNA expression was also determined in transplant islet specific exosomes. Levels are shown as fold expression over the median value for the microarray set. This confirmed that isloet exosome express insulin and FXYD2.

FIG. 23. Twenty highest expressing long RNAs in transplant islet specific exosomes and islet graft tissue showing similar profiles. Levels are shown as expression over median value for the microarray set.

FIG. 24. Twenty highest expressing microRNAs in transplant islet specific exosomes and islet graft showing distinct profiles.

FIG. 25. Twenty highest upregulated transplant islet specific exosomes microRNA compared to islet graft tissue (expression transplant islet specific exosomes/expression islet graft tissue) showing >1000 fold enrichment of certain microRNAs in transplant islet specific exosomes.

FIG. 26. Twenty highest upregulated microRNA in islet graft tissue compared to transplant islet specific exosomes (Expression islet tissue/expression transplant islet specific exosomes) showing down regulation of one of the most abundant islet beta cell specific microRNA, miR-375, in transplant islet specific exosomes (2922 fold).

FIGS. 27A-27D. TISE signal tracked and quantified in the clinical setting of human allogeneic islet transplantation over long term follow-up. A-D) Light scatter and fluorescence signals for patients A (HLA-B13), B (HLA-A2), C (HLA-B8), and D (HLA-B8) over long term follow-up are shown.

FIGS. 28A-28E. A-D) Plasma samples from islet transplant recipients, patients A-D, were analyzed on NanoSight using anti-donor HLA class I specific antibody quantum dot, and the transplant islet exosome signal (primary y axis, blue (top) line) was quantified over long term follow-up (up to 1848 days post-transplant). Recipient plasma [C-peptide (ng/ml) to glucose (mg/dl) ratio]×100 values over the follow-up period is also shown (secondary y axis, black (bottom) line). E) Mean transplant islet exosome signal in patients B, C, and D is shown along with the signal in Patient A separately, as the latter subsequently developed diabetes.

FIGS. 29A-29B. A) NanoSight light scatter and fluorescence analysis of recipient total plasma EVs analyzed for donor specific HLA-A2 signal in a type I diabetic patient undergoing allogeneic islet transplantation. B) NanoSight light scatter and fluorescence analysis of HLA-A2 bound exosomes tested for FXYD2 signal show that post-transplant recipient plasma donor exosomes are positive for islet beta cell marker expression.

FIGS. 30A-30B. A) Western blot analysis of exosome markers CD63 and flotillin-1, and cellular/apoptotic body marker cytochrome C. B) Western blot analysis of the HLA-A2 bound EV fractions analyzed for the presence of endocrine hormones and FXYD2 validated the findings seen in the xenoislet model.

FIG. 31. Daily exogenous insulin requirements in the human recipient during the peri-transplant period.

FIG. 32. RT-PCR analysis of RNA cargo from HLA-A2 bound EV fractions showing expression of insulin, glucagon, somatostatin, and FXYD2 isoforms (γa and γb) in the post-transplant sample.

FIG. 33. Serum C peptide levels for the human recipient showing insulin production by the transplanted islets.

FIG. 34. NanoSight light scatter and fluorescence analysis of total recipient total plasma pool tested for HLA-A2 and FXYD2 signal after islet rejection. Representative six week sample is shown. Islet rejection correlated with loss of donor specific HLA-A2 signal.

FIG. 35. Western blot analysis of the HLA-A2 bound EV fractions from the three different post-islet rejection time points and HLA-A2 positive human plasma. Donor islet culture supernatant EVs and xenoislet graft tissue served as positive controls.

FIG. 36. Western blot analysis of the HLA-A bound EV fractions analyzed for the presence of GAD65. Lane 1: marker, Lane 2: B6 mouse plasma exosomes (negative control), Lane 3: Xenoislet transplant: purified human islet exosomes from recipient mouse plasma, Lane 4: Xenoislet transplant: mouse plasma exosomes unpurified fraction, Lane 5: Xenoislet transplant: purified human islet exosomes from recipient mouse plasma one week after removal of human islet graft, Lane 6: Human plasma exosomes (control), and Lane 7: Xenoislet transplant: removed human islet graft whole cell protein lysate.

FIG. 37. Western blot analysis of the HLA-A bound EV fractions analyzed for the presence of ZnT8. Lane 1: B6 naive mouse plasma exosomes (negative control), Lane 2: Xenoislet transplant: purified human islet exosomes from recipient mouse plasma, Lane 3: Islet culture supernatant exosomes, Lane 4: Xenoislet transplant: purified human islet exosomes from recipient mouse plasma one week after removal of human islet graft, and Lane 5: Human plasma exosomes (control).

FIG. 38. NanoSight light scatter and fluorescence analysis of total recipient total plasma pool tested for HLA-A2. Representative fluorescence for HLA-A positive exosomes is shown in two xenoislet animals without rejection (xenoislet 1, 2). IgG control represents isotype antibody signal in a xenoislet animal, and represents background fluorescence signal.

FIG. 39. Western blot analysis of the HLA-A bound and unbound EV fractions analyzed for the presence of Insulin. Naive mouse plasma and human plasma served as negative controls. Protein from islet graftectomy lysate (labeled as Islet graft) served as positive tissue control.

FIG. 40. Representative glucose curves in 6 animals are shown, with the time of leukocyte injection labeled as lymphocytes.

FIG. 41. Histology of the transplanted islet mass from R-xeno animal is shown. Immunohistochemistry for insulin (red) showed decreased islet mass underneath the renal capsule, along with dense infiltration into the islet graft by T-cells (marked by arrow). Staining for T-cells was performed using anti-CD3 antibody (brown).

FIG. 42. Total plasma exosome numbers were quantified on the NanoSight light scatter mode and expressed as number of nanoparticles/ml per microgram of exosome protein. Box-Whisker plot analysis showed similar total exosome numbers in N-xeno versus R-xeno samples (p=0.91, n=15 in each study arm).

FIG. 43. TISE signal, quantified by normalizing the HLA-A positive exosome signal to total exosome number, was significantly decreased in all the R-xeno animals (p<0.0001). Data is shown in a Box-Whisker plot.

FIG. 44. NanoSight fluorescence of HLA-A bound exosomes from R-xeno plasma showing decreased co-expression of FXYD2.

FIG. 45. Western blot analysis showed decreased levels of FXYD2 and insulin proteins compared to N-xeno sample. Naive mouse plasma HLA-A bound exosomes were included as negative control, and N-xeno islet graft tissue was positive control.

FIG. 46. RT-PCR analysis showed decreased levels of FXYD2 and insulin mRNAs. compared to N-xeno sample. Naive mouse plasma HLA-A bound exosomes were included as negative control, and N-xeno islet graft tissue was positive control.

FIGS. 47A-47D. Proteomic data from mass spectrometry from two N-xeno and three R-xeno TISE samples were analyzed for consistent differences between the two groups. This revealed four proteins: angiopoietin-1 (A), Hsc-70 (B), hemopexin (C), and complement C3 (D) to be differentially expressed. Quantitative normalized value from spectral counting on the Scaffold program software for each protein in the two N-xeno and three R-xeno samples are shown.

FIG. 48. NanoSight fluorescence for HLA-A2 and HLA-B27 showed strong signals in the Donor plasma (positive control) and Recipient post-transplant d4 (day 4) plasma samples, but not in the Recipient pre-transplant sample.

FIG. 49. Western Blot analysis of HLA-A2 bound exosomes analyzed for expression of renal collecting duct apical membrane protein, aquaporin 2.

FIG. 50. NanoSight fluorescence of recipient urine exosome analysis for HLA-A2 presence.

FIG. 51. Western blot analysis of HLA-A2 bound exosomes from urine samples post-transplant day 4 and day 30 showed expression of renal glomerular protein, podocalyxin-1, but the glomerular marker protein was undetectable in the pre-transplant sample.

FIG. 52. Western blot analysis of bound and unbound HLA-A2 exosomes from post-transplant recipient urine showed CD3 expression (Post-transplant day 4, day 30) but not the pre-transplant sample.

FIG. 53. NanoSight fluorescence of Enriched CD3+ exosome subset analyzed for co-expression of helper T-cell (CD4) and cytotoxic T-cell (CD8) surface markers.

FIG. 54. NanoSight fluorescence of HLA-A2 unbound, CD3 unbound urine exosomes analyzed for CD19 (B cell surface marker) expression. Post-transplant day 4 sample (bottom panel) showed CD19 signal but not the Pre-transplant sample.

FIGS. 55A-55B. A) Western blot analysis of exosomes from supernatant media of in vitro cultured islets. B) Western blot analysis of exosomes from supernatant media of in vitro cultured islets from a second patient.

FIGS. 56A-56B. A) RT-PCR analysis of exosomes from supernatant media of in vitro cultured islets. B) RT-PCR analysis of mRNA expression of islet endocrine hormones from in vitro cultured islets.

FIG. 57. Western blot analysis of FXYD2 γA bead bound exosomes show enrichment of insulin containing exosomes in vitro.

FIGS. 58A-58B. A) Western blot analysis of FXYD2 γA bead bound exosomes show enrichment of insulin containing exosomes in vitro. B) RT-PCR analysis of FXYD2 γA bead bound exosomes show enrichment of insulin mRNA containing exosomes in vitro.

FIG. 59. Western blot analysis of FXYD2 γA bead bound exosomes show enrichment of insulin containing exosomes in human plasma.

FIGS. 60A-60B. A) Western blot analysis of FXYD2 γA bead bound exosomes show enrichment of of FXYD2 γA containing exosomes. B) Western blot analysis of FXYD2 γA bead bound exosomes show no enrichment of insulin.

FIGS. 61A-61B. A) Western blot analysis of HLA-A2 bead bound exosomes in a type 1 diabetic patient undergoing single donor islet cell transplantation. B) Western blot analysis of FXYD2 γA bead bound exosomes in a type 1 diabetic patient undergoing single donor islet cell transplantation.

FIG. 62. Kinetics of HLA exosome signal in xenoislet recipients during evolution of acute rejection is shown in scatter plot. Donor sensitized syngeneic leukocytes were infused to induce rejection and HLA exosome signal (squares, y axis) was profiled at following days: 0 (4 hours), 1, 2, 3, 5, 7 (n=8 animals/time point). Controls included athymic mouse, C57BL/6, xenoislet (n=8 for each). HLA exosome signal post-placebo infusion shown as black circles. Mean fasting glucose shown as solid line. Summarized data (mean±SD) from two independent experiments is shown.

FIG. 63. Daily intraperitoneal glucose tolerance tests are shown (mean values, n=8 per time point).

FIG. 64. Receiver operating characteristic curves for HLA exosome signal (1), total plasma exosome quantity (2), and median exosome size (3) is shown.

FIG. 65. Representative islet graft histology (1 of 4) is shown for Day 1, 2, 3, and 5. H&E and immunohistochemistry for insulin (brown, red arrow) and T cells (CD3, pink, black arrow) are shown.

FIG. 66. Plasma T cell exosome signal (CD3 signal) is shown (mean±SD). Compared to xenoislet.

### DETAILED DESCRIPTION

Tissue specific exosome profiling from a subject's bodily fluids has broad clinical implications for development of non-invasive biomarker platforms for monitoring the status of an organ/tissue or disease. For example, transplant tissue specific exosome from a recipient's bodily fluid can be used to monitor transplanted organ or tissue rejection and/or injury. Similarly, tissue specific EV in a subject's bodily fluid can be used to diagnose or monitor disease.

The present disclosure provides techniques related to the use of one or more biomarkers identified herein to monitor the conditional state of a transplanted organ or tissue in a subject. To this end, the present disclosure provides applications for isolating and analyzing microvesicles released from a transplanted donor organ or tissue from the bodily fluids of a subject. In certain embodiments, this method of detecting, purifying, and profiling transplant tissue specific exosomes from recipient bodily fluids can be applied to all transplanted tissues and organs.

In certain embodiments, the transplanted tissue can be pancreatic islets. In certain embodiments, the transplanted tissue can be islet beta cells. In certain embodiments, the bodily fluid can be blood.

In certain embodiments, the transplanted organ can be kidney. In certain embodiments, the bodily fluid can be blood and/or urine.

Accordingly, this disclosure provides for methods and kits for determining the presence and/or levels of one or more biomarkers for organ or tissue rejection/injury in a biological sample of a subject, and methods for using the presence and/or levels of such biomarkers to predict or diagnose organ or tissue rejection/injury in a subject, and to select or modify a therapeutic regimen for a subject with a transplanted organ or tissue. Exemplary biomarkers that can be used in the methods of the present disclosure are presented below.

Furthermore, the effectiveness of immunosuppression therapy can be monitored by evaluating the presence and/or levels of the one or more biomarkers over the course of a therapy, and decisions can be made regarding the type, duration and course of therapy based on these evaluations.

The present disclosure also provides techniques related to the use of one or more biomarkers identified herein to diagnose or monitor at least one disease in a subject. To this end, the present disclosure provides applications for isolating and analyzing microvesicles released into the bodily fluids of the subject. In certain embodiments, such isolation is accomplished via exosome selection based on the presence of one or more exosome surface proteins.

Accordingly, this disclosure provides for methods and kits for determining the presence and/or levels of one or more biomarkers for a disease in a biological sample of a subject, and methods for using the presence and/or levels of such biomarkers to predict, diagnose, or monitor the disease in a subject, and to select or modify a therapeutic regimen for a subject. The biomarkers that can be used in the methods of the present disclosure are set forth below.

Furthermore, the effectiveness of a disease therapy can be monitored by evaluating the presence and/or levels of the one or more biomarkers over the course of a therapy, and decisions can be made regarding the type, duration, and course of therapy based on these evaluations.

In certain embodiments, the disease is a pancreatic disorder. In certain embodiments, the disease is pancreatitis. In certain embodiments the disease is a metabolic disease. In certain embodiments, the disease is type I diabetes, metabolic syndrome, type II diabetes, and gestational diabetes. In certain embodiments, diabetes is type I diabetes. In certain embodiments, the disease is a kidney disorder.

Current medical practices rely on clinical parameters to monitor the function of beta islet cells. Detection of either donor organ- or tissue-derived or native microvesicles circulating in a biological fluid of a recipient, as described herein, provides for a non-invasive, time-sensitive, direct, and reliable biomarker assay to determine the conditional state of beta islet cells.

Given the role of islet beta cells in glucose metabolism, and central role that insulin (released by beta cells) plays in nutrition metabolism, the beta cell exosomes platform from a subject's bodily fluid (e.g., plasma) may provide for a noninvasive biomarker assay for nutridynamics.

### Definitions

As used herein, “transplantation” refers to the process of taking a tissue or organ, called a “transplant” or “graft” from one subject and placing it or them into a (usually) different subject. The subject who provides the transplant is called the “donor” and the subject who received the transplant is called the “recipient.” An organ, or graft, transplanted between two genetically different subjects of the same species is called an “allograft.” A graft transplanted between subjects of different species is called a “xenograft.” Examples of transplanted organs that can be monitored by the methods disclosed herein include, but are not limited to, heart, lungs, kidney, liver, islets, and pancreas.

As used herein, the term “transplant rejection,” is defined as functional and/or structural deterioration of an organ or tissue. Transplant rejection can include functional and/or structural deterioration due to an active immune response expressed by the recipient, and independent of non-immunologic causes of organ or tissue dysfunction. Transplant rejection can include donor organ or tissue injury, such as an infection of the transplant organ or tissue.

As used herein, the term “biomarker” refers to a marker (e.g., an expressed gene, including mRNA, microvesicle pool profile, and/or protein) that allows detection of a disease in an individual, including detection of disease in its early stages. Biomarkers, as used herein, include microvesicles (e.g., exosomes), nucleic acid, and/or protein markers or combinations thereof. In certain non-limiting embodiments, the expression level of a biomarker as determined by mRNA and/or protein levels in a biological sample from an individual to be tested is compared with respective levels in a biological sample from the same individual, another healthy individual, or from the transplanted tissue or organ. In certain non-limiting embodiments, the presence or absence of a biomarker as determined by mRNA and/or protein levels in a biological sample from an individual to be tested is compared with the respective presence or absence in a biological sample from the same individual, another healthy individual, or from the transplanted tissue or organ. In certain non-limiting embodiments, the presence or absence of a biomarker in a biological sample of a subject is compared to a reference control.

The terms “reference sample” or “reference,” as used interchangeably herein, refers to a control for a biomarker that is to be detected in a biological sample of a subject. For example, a control can be the level of a biomarker from a healthy individual that underwent organ or tissue transplantation, wherein the organ or tissue is in a tolerance state. In certain embodiments, a reference can be the level of a biomarker detected in a healthy individual that did not undergo an organ or tissue transplant. In certain embodiments, a control can be the level of a biomarker from a healthy individual that underwent treatment for a disease, wherein the healthy individual is non-symptomatic. In certain embodiments, a reference can be the level of a biomarker detected in a healthy that has never had the disease. In certain embodiments, a control can be the level of a biomarker from a healthy individual that underwent diabetes treatment, wherein the healthy individual is normoglycemic. In certain embodiments, a reference can be the level of a biomarker detected in a healthy non-diabetic individual. In other embodiments, the reference can be a predetermined level of a biomarker that indicates transplanted organ or tissue tolerance. In other embodiments, the reference can be a predetermined level of a biomarker that indicates transplanted organ or tissue rejection/tolerance. In other embodiments, the reference can be a predetermined level of a biomarker that indicates a subject is not diabetic. In certain embodiments, the reference can be an earlier sample taken from the same subject. In certain embodiments, the reference can be a sample taken from the transplanted organ or tissue, either before or after transplantation.

As used herein, the term “biological sample” refers to a sample of biological material obtained from a subject, e.g., a human subject, including a biological fluid, e.g., blood, plasma, scrum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, bronchoalveolar fluid, biliary fluid and combinations thereof. In certain non-limiting embodiments, the presence of one or more biomarkers is determined in a blood sample obtained from a subject. In certain non-limiting embodiments, the presence of one or more biomarkers is detected in a urine sample obtained from a subject.

The term “patient” or “subject,” as used interchangeably herein, refers to any warm-blooded animal, e.g., a human. Non-limiting examples of non-human subjects include non-human primates, dogs, cats, mice, rats, guinea pigs, rabbits, fowl, pigs, horses, cows, goats, sheep, etc.

The term “microvesicle” as used herein, refers to vesicles that are released from a cell. In certain embodiments, the microvesicle is a vesicle that is released from a cell by exocytosis of intracellular multivesicular bodies. In certain embodiments, the microvesicles can be exosomes. In certain embodiments, the microvesicles can be in the size range from about 30 nm to 1000 nm.

### Prognostic and Diagnostic Methods

Certain embodiments of the present disclosure relate to methods for assessing the conditional state of a transplanted organ or tissue in a subject. In certain embodiments, the method of assessing the conditional state of a transplanted organ or tissue in a subject comprises diagnosing or monitoring the transplanted organ or tissue for rejection and/or injury. In certain embodiments, a method for diagnosing or monitoring transplanted organ or tissue rejection and/or injury in a subject is disclosed, wherein the method includes: (a) obtaining a biological sample from the subject; (b) isolating, purifying, and/or identifying one or more donor organ- or tissue-derived microvesicles from the biological sample; (c) detecting the presence or level of one or more biomarkers from the pool of isolated, purified, or identified microvesicles; and (d) diagnosing organ or tissue rejection and/or injury in the subject, wherein the change in the presence and/or level of the one or more biomarkers indicates transplant organ or tissue rejection and/or injury in the subject. In certain embodiments, the change in presence and/or level is determined by comparing the sample to a reference sample. Exemplary biomarkers that can be used in connection with certain embodiments of the present disclosure are presented below.

In certain embodiments, a method for diagnosing or monitoring transplanted organ or tissue rejection in a subject is disclosed, wherein the method includes: (a) obtaining a biological sample from a subject; (b) isolating, purifying, and/or identifying one or more donor organ- or tissue-derived microvesicles from the biological sample; and (c) diagnosing organ or tissue rejection and/or injury in the subject, wherein the change in the size and/or number of the microvesicles indicates transplant organ or tissue rejection and/or injury in the subject. In certain embodiments, the change in size and/or number is determined by comparing the sample to a reference sample. In certain embodiments, the microvesicles can be exosomes.

In certain embodiments, a method for diagnosing or monitoring transplanted organ or tissue rejection in a subject is disclosed, wherein the method includes: (a) obtaining a biological sample from a subject; (b) isolating, purifying, and/or identifying one or more donor organ- or tissue-derived microvesicles from the biological sample; and (c) diagnosing organ or tissue rejection and/or injury in the subject, wherein the change in the size and/or number of the microvesicles indicates a change in the conditional state of a transplanted organ or tissue of the subject. In certain embodiments, the change in size and/or number is determined by comparing the sample to a reference sample. In certain embodiments, the microvesicles can be exosomes.

In certain embodiments, a method for diagnosing or monitoring transplanted organ or tissue rejection in a subject is disclosed, wherein the method includes: (a) obtaining a biological sample from a subject; (b) isolating, purifying, and/or identifying one or more biomarkers from the biological sample; and (c) diagnosing organ or tissue rejection and/or injury in the subject, wherein the change in the presence and/or level of the one or more biomarkers indicates transplant organ or tissue rejection and/or injury in the subject. In certain embodiments, the change in presence and/or level is determined by comparing the sample to a reference sample.

In certain embodiments, a method for diagnosing or monitoring transplanted organ or tissue rejection in a subject is disclosed, wherein the method includes: (a) obtaining a biological sample from a subject; (b) isolating, purifying, and/or identifying one or more biomarkers from the biological sample; and (c) diagnosing organ or tissue rejection and/or injury in the subject, wherein the change in the presence and/or level of the one or more biomarkers indicates a change in the conditional state of a transplanted organ or tissue of the subject. For example, a change in the presence and/or level of a biomarker from a biological sample can indicate that the transplanted organ or tissue is being tolerated and/or maintained. In certain embodiments, the change in presence and/or level is determined by comparing the sample to a reference sample.

In certain embodiments, the transplanted tissue can be pancreatic islet cells. In certain embodiments, the transplanted tissue can be islet beta cells. In certain embodiments, the transplanted tissue can be cultured islet beta cells. In certain embodiments, the transplanted organ can be a kidney. In certain embodiments, the microvesicles can be exosomes.

Certain embodiments of the present disclosure relate to methods for assessing the disease state of a subject. In certain embodiments, a method for predicting the risk of, diagnosing, or monitoring a disease in a subject is disclosed, wherein the method includes: (a) obtaining a biological sample from the subject; (b) isolating, purifying, and/or identifying one or more subject organ- or tissue-derived microvesicles from the biological sample; (c) detecting the presence or level of one or more biomarkers from the pool of isolated, purified, or identified microvesicles; and (d) predicting or diagnosing the disease in the subject, wherein the change in the presence and/or level of the one or more biomarkers indicates the subject has or will develop the disease. In certain embodiments, the change in presence and/or level is determined by comparing the sample to a reference sample. Exemplary biomarkers that can be used in connection with certain embodiments of the present disclosure are presented below.

In certain embodiments, a method for predicting the risk of, diagnosing, or monitoring a disease in a subject is disclosed, wherein the method includes: (a) obtaining a biological sample from a subject; (b) isolating, purifying, and/or identifying one or more subject organ- or tissue-derived microvesicles from the biological sample; and (c) predicting the risk of or diagnosing the disease in the subject, wherein the change in the size and/or number of the microvesicles indicates the subject has or will develop the disease. In certain embodiments, the change in size and/or number is determined by comparing the sample to a reference sample. In certain embodiments, the microvesicles can be exosomes.

In certain embodiments, a method for predicting the risk of, diagnosing, or monitoring a disease in a subject is disclosed, wherein the method includes: (a) obtaining a biological sample from a subject; (b) isolating, purifying, and/or identifying one or more biomarkers from the biological sample; and (c) predicting the risk or diagnosing the disease in the subject, wherein the change in the presence and/or level of the one or more biomarkers indicates the subject has or will develop the disease. In certain embodiments, the change in presence and/or level is determined by comparing the sample to a reference sample.

In certain embodiments, the disease is a pancreatic disorder. In certain embodiments, the disease is pancreatitis. In certain embodiments the disease is a metabolic disease. In certain embodiments, the disease is diabetes. In certain embodiments, diabetes is type I diabetes, type II diabetes, or gestational diabetes. In certain embodiments, diabetes is type I diabetes.

In certain embodiments, the tissue can be pancreatic islet cells. In certain embodiments, the tissue can be islet beta cells. In certain embodiments, the microvesicles can be exosomes.

In certain embodiments, the methods for detection of one or more biomarkers can be used to monitor the response in a subject to prophylactic or therapeutic treatment (for example, diabetes therapy or immunosuppression therapy to prevent organ or tissue rejection). In certain non-limiting embodiments, the disclosed subject matter further provides a method of treatment including measuring the presence and/or level of one or more biomarkers of the present disclosure in a subject at a first time point, administering a therapeutic agent, re-measuring the one or more biomarkers at a second time point, comparing the results of the first and second measurements and optionally modifying the treatment regimen based on the comparison. In certain embodiments, the first time point is prior to an administration of the therapeutic agent, and the second time point is after said administration of the therapeutic agent. In certain embodiments, the first time point is prior to the administration of the therapeutic agent to the subject for the first time. In certain embodiments, the dose (defined as the quantity of therapeutic agent administered at any one administration) is increased or decreased in response to the comparison. In certain embodiments, the dosing interval (defined as the time between successive administrations) is increased or decreased in response to the comparison, including total discontinuation of treatment. In addition, the method of the present disclosure can be used to determine the efficacy of immunosuppression therapy, wherein a change in the level and/or presence of a biomarker in a biological sample of a subject can indicate that the immunosuppressive therapy regimen can be increased, maintained, reduced, or stopped.

Additionally, the method of the present disclosure can be used to determine the efficacy of a disease therapy, wherein a change in the level and/or presence of a biomarker in a biological sample of a subject can indicate that the therapy regimen can be increased, maintained, reduced, or stopped. In certain embodiments, the disease is a pancreatic disorder. In certain embodiments, the disease is pancreatitis. In certain embodiments the disease is a metabolic disease. In certain embodiments, the disease is diabetes. In certain embodiments, diabetes is type I diabetes, type II diabetes, or gestational diabetes. In certain embodiments, diabetes is type I diabetes.

In certain embodiments, the one or more biomarkers can be detected in blood (including plasma or serum) or in urine, or alternatively at least one biomarker can detected in one sample, e.g., the blood, plasma or serum, and at least one other biomarker is detected in another sample, e.g., in urine. The step of collecting a biological sample can be carried out either directly or indirectly by any suitable technique. For example, a blood sample from a subject can be carried out by phlebotomy or any other suitable technique, with the blood sample processed further to provide a serum sample or other suitable blood fraction.

In certain embodiments, the information provided by the methods described herein can be used by the physician in determining the most effective course of treatment (e.g., preventative or therapeutic). A course of treatment refers to the measures taken for a patient after the assessment of increased risk for disease (e.g., diabetes) or organ or tissue rejection is made. For example, when a subject is identified to have an increased risk of disease (e.g., diabetes) or organ or tissue rejection, the physician can determine whether frequent monitoring for biomarker detection is required as a prophylactic measure.

### Biomarkers

In certain embodiments, the biomarker is a pool of one or more organ- or tissue-derived microvesicles (e.g., from the subject and/or a donor).

In certain embodiments, the disclosure provides for methods for assessing the conditional state or status of a transplanted organ or tissue in a subject, comprising isolating microvesicles from the donor organ or tissue in a biological sample of the subject, determining the size and/or number of isolated microvesicles, wherein a change in the size and/or number of the microvesicles compared to a reference is an indication of transplant organ or tissue rejection and/or injury in the subject. In certain embodiments, the change in a physical characteristic, e.g., the size and/or number, of the microvesicles, from a biological sample of a subject, compared to a reference is indicative of a change in the conditional state or status of the transplanted organ or tissue in the subject. In certain embodiments, the change in the size and/or number of microvesicles compared to a reference is detected before clinical signs of the return of the disease treated by the transplantation is detected. In certain embodiments, the change in the size and/or number of microvesicles compared to a reference is detected before the clinical onset of hyperglycemia.

In certain embodiments, the transplanted tissue can be pancreatic islet cells. In certain embodiments, the transplanted tissue can be islet beta cells. In certain embodiments, the transplanted tissue can be cultured islet beta cells. In certain embodiments, the transplanted organ can be a kidney. In certain embodiments, the microvesicles can be exosomes.

In certain embodiments, an increase or decrease of at least about 1.5 times, at least about 2 times, at least about 2.5 times, at least about 3 times, at least about 3.5 times, at least about 4.0 times, at least about 4.5 times, or at least about 5 times the number of microvesicles as compared to a reference sample is indicative of a change in the conditional state of the transplanted organ or tissue in the subject or an indication of transplant organ or tissue rejection and/or injury in the subject. In certain embodiments, an increase or decrease of at least about 2 times the number of microvesicles as compared to a reference sample is indicative of a change in the conditional state of the transplanted organ or tissue in the subject or an indication of transplant organ or tissue rejection and/or injury in the subject. In certain embodiments, an increase or decrease of at least about 2.5 times the number of microvesicles as compared to a reference sample is indicative of a change in the conditional state of the transplanted organ or tissue in the subject or an indication of transplant organ or tissue rejection and/or injury in the subject. In certain embodiments, an increase or decrease of at least about 0.5 times the number of microvesicles as compared to a reference sample is indicative of a change in the conditional state of the transplanted organ or tissue in the subject or an indication of transplant organ or tissue rejection and/or injury in the subject. In certain embodiments, an increase or decrease of at least about 0.3 times the number of microvesicles as compared to a reference sample is indicative of a change in the conditional state of the transplanted organ or tissue in the subject or an indication of transplant organ or tissue rejection and/or injury in the subject. In certain embodiments, an increase or decrease of at least about 0.2 times, at least about 0.3 times, at least about 0.4 times, at least about 0.5 times, at least about 0.6 times the number of microvesicles as compared to a reference sample is indicative of a change in the conditional state of the transplanted organ or tissue in the subject or an indication of transplant organ or tissue rejection and/or injury in the subject. In certain embodiments, an increase or decrease of between about 0.2 to about 0.5, about 0.22 to about 0.48, about 0.24 to about 0.46, about 0.26 to about 0.44, about 0.28 to about 0.42, about 0.3 to about 0.4, about 0.32 to about 0.38, about 0.34 to about 0.36, about 0.25 to about 5, about 0.5 to about 4.75, about 0.75 to about 4.5, about 1 to about 4.25, about 1.25 to about 4, about 1.5 to about 3.75, about 1.75 to about 3.5, about 2 to about 3.25, about 2.25 to about 3, or about 2.5 to about 2.75 times the number of microvesicles as compared to a reference sample is indicative of a change in the conditional state of the transplanted organ or tissue in the subject or an indication of transplant organ or tissue rejection and/or injury in the subject. In certain embodiments, it is a decrease in the number of microvesicles that is indicative of a change in the conditional state of the transplanted organ or tissue in the subject or an indication of transplant organ or tissue rejection and/or injury in the subject. In certain embodiments, it is a increase in the number of microvesicles that is indicative of a change in the conditional state of the transplanted organ or tissue in the subject or an indication of transplant organ or tissue rejection and/or injury in the subject.

In certain embodiments, the biomarker is a pool of one or more organ- or tissue-derived microvesicles (from the donor and/or subject), e.g., exosomes, wherein a change in a physical characteristic, e.g., the size and/or number, and/or the profile of the microvesicles is prognostic of and/or indicative of donor organ or tissue rejection and/or injury or disease in a subject.

In certain embodiments, the biomarker is the change in the number of donor organ specific exosomes. In certain embodiments, the biomarker is a decrease in the number of donor organ specific exosomes. In certain embodiments, the donor organ specific exosomes are donor islet specific exosomes. In certain embodiments, the donor organ specific exosomes are donor kidney specific exosomes.

In certain embodiments, the biomarker is the change in the number of recipient specific exosomes. In certain embodiments, the biomarker is an increase or decrease in the number of recipient specific exosomes.

In certain embodiments, the disclosure provides for methods for assessing the diabetic state of a subject and the islet beta cell mass, comprising isolating microvesicles from an organ or tissue in a biological sample of the subject, determining the size and/or number of the isolated microvesicles, wherein a change in the size and/or number of the microvesicles compared to a reference is an indication of diabetes or dysfunctional beta cell function in the subject. In certain embodiments, the tissue can be pancreatic islet cells. In certain embodiments, the tissue can be islet beta cells. In certain embodiments, the microvesicles can be exosomes.

In certain embodiments, the biomarker is a protein isolated from a pool of one or more isolated organ- or tissue-derived microvesicles from the subject or transplant recipient.

In certain embodiments, the disclosure provides for methods for assessing the conditional state or status of a transplanted organ or tissue in subject, comprising isolating microvesicles from the donor organ or tissue from a biological sample of the subject, isolating the one or more protein biomarkers from the donor organ- or tissue-derived microvesicles, wherein a change in the level and/or presence of the protein biomarker compared to a reference sample is an indication that the transplanted organ or tissue is being rejected and/or is injured. In certain embodiments, the change in the level and/or presence of a protein biomarker, from a biological sample of a subject, compared to a reference is indicative of a change in the conditional state or status of the transplanted organ or tissue in the subject. In certain embodiments, the transplanted tissue can be pancreatic islet cells. In certain embodiments, the transplanted tissue can be islet beta cells. In certain embodiments, the transplanted tissue can be cultured islet beta cells. In certain embodiments, the microvesicles can be exosomes.

In certain embodiments, the disclosure provides for assessing the diabetic state of a subject, comprising isolating microvesicles from the organ or tissue from a biological sample of the subject, isolating the one or more protein biomarkers from the organ- or tissue-derived microvesicles, wherein a change in the level and/or presence of the protein biomarker compared to a reference sample is an indication that the subject has or will develop diabetes. In certain embodiments, the change in the level and/or presence of a protein biomarker, from a biological sample of a subject, compared to a reference is indicative of a change in the diabetic state of the subject. In certain embodiments, the tissue can be pancreatic islet cells. In certain embodiments, the tissue can be islet beta cells. In certain embodiments, the microvesicles can be exosomes.

In certain embodiments, the presence of the protein is detected using a reagent which specifically binds with the protein. For example, the reagent can be an antibody, an antibody derivative, an antigen-binding antibody fragment and a non-antibody peptide which specifically binds the protein. In certain embodiments, the antibody or antigen-binding antibody fragment is a monoclonal antibody or antigen-binding fragment thereof, or a polyclonal antibody or antigen-binding fragment thereof. In certain embodiments, the protein biomarker can be detected by biophysical platforms such as mass spectrometry.

As outlined in detail in the examples disclosed below, islet cell-derived exosomes express FXYD2 (including the γa isoform), insulin, GAD65, and/or ZnT8 protein, which allow for islet cell-specific characterization of exosomes.

In certain embodiments, FXYD2, FXYD2γ2a, and/or FXY2γb enriched exosomes/microvesicles allow for characterization of islet/islet beta cell specific exosomes from a subject's bodily fluid.

As outlined in detail in the examples disclosed below, T-cell-derived exosomes express cluster of differentiation (CD) proteins CD3, CD4, and/or CD8, which allow for T-cell-specific characterization of exosomes.

In certain embodiments, CD3, CD4, and/or CD8 enriched exosomes/microvesicles allow for characterization of T-cell specific exosomes from a subject's bodily fluid.

As outlined in detail in the examples disclosed below, B-cell-derived exosomes express cluster of differentiation (CD) proteins CD5, CD19, CD20, CD22, CD23, CD24, CD27 and/or CD38, which allow for B-cell-specific characterization of exosomes.

In certain embodiments, CD5, CD19, CD20, CD22, CD23, CD24, CD27 and/or CD38 enriched exosomes/microvesicles allow for characterization of B-cell specific exosomes from a subject's bodily fluid.

In certain embodiments, the biomarker can be heat shock cognate protein 71 (Hsc-70), angiopoietin-1, hemopexin, and/or complement C3 isolated from a pool of one or more organ- or tissue-derived microvesicles (from the donor and/or subject). In certain embodiments, the biomarker can be at least one, at least two, at least three, or at least four of heat shock cognate protein 71 (Hsc-70), angiopoietin-1, hemopexin, and complement C3. In certain embodiments, the change in the expression levels of heat shock cognate protein 71 (Hsc-70), angiopoietin-1, hemopexin, and/or complement C3 in the pool of one or more organ- or tissue-derived microvesicles can be a biomarker for the rejection of the donor organ or tissue. In certain embodiments, the change in expression of heat shock cognate protein 71 (Hsc-70) is a decrease in expression as compared to a control (e.g., before rejection or normal patient). In certain embodiments, the change in expression of angiopoietin-1 is a decrease in expression as compared to a control (e.g., before rejection or normal patient). In certain embodiments, the change in expression of hemopexin is an increase in expression as compared to a control (e.g., before rejection or normal patient). In certain embodiments, the change in expression of complement C3 is an increase in expression as compared to a control (e.g., before rejection or normal patient).

In certain embodiments, the biomarker can also be a nucleic acid or portion thereof, e.g., a mRNA, DNA, cDNA miRNA, snoRNA, scaRNA, lncRNA, or piRNA isolated from a pool of one or more organ- or tissue-derived microvesicles (e.g., from the subject or a donor).

In certain embodiments, the disclosure provides for methods for assessing the conditional state or status of a transplanted organ or tissue in subject, comprising isolating donor organ- or tissue-derived microvesicles from a biological sample of the subject, isolating the one or more nucleic acid biomarkers from the donor organ- or tissue-derived microvesicles, wherein a change in the level and/or presence of the nucleic acid biomarker compared to a reference sample is an indication that the transplanted organ or tissue is being rejected. In certain embodiments, the change in the level and/or presence of a nucleic acid biomarker, from a biological sample of a subject, compared to a reference is indicative of a change in the conditional state or status of the transplanted organ or tissue in the subject. In certain embodiments, the nucleic acid biomarker can be mRNA, DNA, cDNA miRNA, snoRNA, scaRNA, lncRNA, or piRNA. In certain embodiments, detecting a transcribed polynucleotide includes amplifying the transcribed polynucleotide. In certain embodiments, the nucleic acid biomarker can be detected by RT-PCR, microarray analysis, or Q-PCR

In certain embodiments, the disclosure provides for methods for assessing the diabetic state of a subject, comprising isolating islet beta cell derived microvesicles from a biological sample of the subject, isolating the one or more nucleic acid biomarkers from the organ- or tissue-derived microvesicles, wherein a change in the level and/or presence of the nucleic acid biomarker compared to a reference sample is an indication that the subject has or will develop diabetes. In certain embodiments, the change in the level and/or presence of a nucleic acid biomarker, from a biological sample of a subject, compared to a reference is indication that the subject has or will develop diabetes. In certain embodiments, the nucleic acid biomarker can be miRNA. In certain embodiments, detecting a transcribed polynucleotide includes amplifying the transcribed polynucleotide. In certain embodiments, the nucleic acid biomarker can be detected by RT-PCR or microarray analysis. In certain embodiments, the disclosure provides method for purifying islet beta cell exosomes and changes in their quantity, size, or proteomic and RNA profiles as nutritional biomarker. Such platforms have applications in diagnosing and monitoring epidemic conditions associated with metabolic dysfunction such as morbid obesity, metabolic syndrome, and type II diabetes.

As outlined in detail in the examples disclosed below, islet cell-derived exosomes express FXYD2 γa, insulin, miR-8075, miR-3613-3p, miR-6089, miR-4668-5p, miR-6090, miR-3960, miR-5787, miR-4508, miR-6732-5p, miR-191-5p, miR-486-5p, miR3613-5p, miR-6087, miR-23a-3p, miR-1281, miR-7704, miR-1469, miR4787-5p, miR-16-5p, and miR-638 nucleotides, which allow for islet cell-specific characterization of exosomes. The examples also demonstrate that miRNA-191-5p, miRNA-23a-3p, miRNA16-5p, and miR-24-3p are differentially expressed in the islet cell-based exosomes as compared to the tissue graft.

### Biomarker Detection

Biomarkers used in the methods of the disclosure can be identified in a biological sample using any method known in the art. Biomarkers can be microvesicles and/or nucleic acids and/or proteins that reside on the surface or within the microvesicles. The microvesicles, e.g., exosomes, can be isolated from a biological sample and analyzed using any method known in the art. The nucleic acid sequences, fragments thereof, and proteins, and fragments thereof, can be isolated and/or identified in a biological sample using any method known in the art.

### Microvesicle Isolation Techniques

Circulating subject- or donor-organ or tissue derived microvesicles can be isolated from a subject by any means known in the art and currently available. Circulating subject- or donor-organ or tissue derived microvesicles can be isolated from a biological sample obtained from a subject, such as a blood sample, or other biological fluid. In certain embodiments, the microvesicles can be exosomes.

There are several capture and enrichment platforms that are known in the art and currently available. For example, microvesicles can be isolated by a method of differential centrifugation as described by Raposo et al., 1996. Additional methods include anion exchange and/or gel permeation chromatography as described in U.S. Pat. Nos. 6,899,863 and 6,812,023. Methods of sucrose density gradients or Organelle electrophoresis are described in U.S. Pat. No. 7,198,923. A method of magnetic activated cell sorting (MACS) is described in Taylor and Gercel-Taylor, 2008. A method of nanomembrane ultrafiltration concentrator is described in Cheruvanky et al., 2007. Microvesicles can be identified and isolated from a biological sample of a subject by a newly developed microchip technology that uses a unique microfluidic platform to efficiently and selectively separate microvesicles (Nagrath et al., 2007). This technology can be adapted to identify and separate microvesicles using similar principles of capture and separation.

In certain embodiments, the microvesicles isolated from a biological sample are enriched for those originating from a specific cell type, for example, but not limited to, lung, pancreas, stomach, intestine, bladder, kidney, ovary, testis, skin, colorectal, breast, prostate, brain, esophagus, liver, placenta or fetus cells. In certain embodiments, the microvesicles isolated from a biological sample are from pancreatic islet cells. In certain embodiments, the microvesicles isolated from a biological sample are from islet beta cells.

In addition, the microvesicles often carry surface molecules (e.g., antigens) that can be used to identify, isolate and/or enrich for microvesicles from a specific cell type (Al-Nedawi et al., 2008; Taylor and Gercel-Taylor, 2008). For example, the surface antigen FXYD2 which is specific to microvesicles from beta cells, but not of other pancreatic islet cell and exocrine cell origin. Also, the surface antigen epithelial-cell-adhesion-molecule (EpCAM), is specific to microvesicles from cells of lung, colorectal, breast, prostate, head and neck, and hepatic origin, but not of hematological cell origin (Balzar et al., 1999; Went et al., 2004). In another example, the surface antigen CD24 is a glycoprotein specific to urine microvesicles (Keller et al., 2007).

In certain embodiments, the microvesicles isolated from a biological sample are enriched for those released from the donor organ or tissue through the use of the Major histocompatibility complex (MHC) proteins (such as, but not limited to HLA-A, HLA-B, HLA-C, HLA-DP, HLA-DQ, HLA-DR or a combination thereof and related subclasses) that reside on the surface of the microvesicles. Therefore, microvesicles originating from distinct cell populations can be analyzed for their nucleic acid content. In certain embodiments, the microvesicles can be isolated from a biological sample of a subject and enriched for those originating from an organ or tissue in the subject or an organ or tissue transplanted into the subject. For example, a donor HLA profile can be compared to a recipient HLA profile, and any HLA proteins specific for the donor or recipient can be used to identify or purify exosomes specific to either the donor or recipient (see Example 5 as an example).

The isolation of microvesicles from specific cell types and/or organs can be accomplished, for example, by using antibodies, aptamers, aptamer analogs or molecularly imprinted polymers specific for a desired surface antigen. In certain embodiments, the surface antigen is specific for a cell type of a specific organ or tissue. One example of a method of microvesicle separation based on cell surface antigen is provided in U.S. Pat. No. 7,198,923. As described in, e.g., U.S. Pat. Nos. 5,840,867 and 5,582,981, WO12003/050290 and a publication by Johnson et al. (Johnson et al., 2008), aptamers and their analogs specifically bind surface molecules and can be used as a separation tool for retrieving cell type-specific microvesicles. Molecularly imprinted polymers also specifically recognize surface molecules as described in, e.g., U.S. Pat. Nos. 6,525,154, 7,332,553 and 7,384,589 and Bossi et al., 2007 and are a tool for retrieving and isolating cell type-specific microvesicles. In certain embodiments, microvesicles can be isolated based on the MHC complex residing on the surface of the microvesicles. In certain embodiments, microvesicles can be isolated based on the FXYD2 isoform (e.g., γa and γb) residing on the surface of the microvesicles. In certain embodiments, antibodies, aptamers, aptamer analogs to FXYD2, FXYD2γa, or FXYD2γb can be used to enrich the micovesicles.

In certain embodiments, the microvesicles can be enriched utilizing one technique and then further enriched using a second technique. For example, in certain embodiments, the microvesicles can be enriched utilizing antibodies, aptamers, aptamer analogs to MHC, and that pool can further be enriched by utilizing antibodies, aptamers, aptamer analogs to FXYD2, FXYD2γa, or FXYD2γb.

In certain non-limiting embodiments, high exclusion limit agarose-based gel chromatography can be utilized to isolate plasma microvesicles (Taylor et al., 2005). For example, to isolate the total vesicle fraction, the plasma sample can be fractionated using a 2.5×30 cm Sepharose 2B column, run isocratically with PBS, and the elution can be monitored by absorbance at 280 nm. The fractions comprising microvesicles can be concentrated to 2 ml using an Amicon ultrafiltration stirred cell with a 500K Dalton cut-off membrane and can used for the affinity separation of organ- or tissue-specific microvesicles subpopulations. Since microvesicles within the circulation are generated from multiple cell types, affinity based approaches can be used to specifically purify subsets of microvesicles (Taylor et al., 2005). For immunosorbent isolation of organ or tissue derived microvesicle populations, plasma microvesicles can be selectively incubated with antibodies specific for a microvesicle surface protein (e.g., FXYD2 or the donor's MHC profile) coupled with magnetic microbeads. After incubation for 2 hours at 4° C., the magnetic bead complexes can be placed in the separator's magnetic field and the unbound microvesicles can be removed with the supernatant. The bound donor-specific microvesicle subsets can be recovered and diluted in IgG elution buffer (Pierce Chemical Co), centrifuged and resuspended in PBS. Donor microvesicle number and size distribution can be determined using the NanoSight NS300. Additional methods to isolate microvesicles include, but are not limited to, ultracentrifugation and sucrose gradient-based ultracentrifugation. In certain embodiments, the microvesicle isolation kit, ExoQuick™, and/or the Exo-Flow™ system from System Bioscience, Inc. can be used.

### Protein Detection Techniques

In certain embodiments, the biomarker is a protein, present on the surface and/or within the subject or donor organ- or tissue-specific isolated microvesicles, e.g., exosomes. Proteins can be isolated from a microvesicle using any number of methods, which are well-known in the art, the particular isolation procedure chosen being appropriate for the particular biological sample.

Methods for the detection of protein biomarkers are well known to those skilled in the art, and include but are not limited to mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein. Antibody arrays or protein chips can also be employed, see for example U.S. Patent Application Nos: 20030013208A1; 20020155493A1, 20030017515 and U.S. Pat. Nos. 6,329,209 and 6,365,418, herein incorporated by reference in their entirety.

ELISA and RIA procedures can be conducted such that a biomarker standard is labeled (with a radioisotope such as 125I or 35S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In certain embodiments, the method for measuring biomarker expression includes the steps of: contacting a biological sample, e.g., blood, with an antibody or variant (e.g., fragment) thereof which selectively binds the biomarker, and detecting whether the antibody or variant thereof is bound to the sample. The method can further include contacting the sample with a second antibody, e.g., a labeled antibody. The method can further include one or more steps of washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited, but can be selected from the members of the oxidase group, for example. These catalyze production of hydrogen peroxide by reaction with their substrates, and glucose oxidase is often used for its good stability, case of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a biomarker according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including 125I, horseradish peroxidase and alkaline phosphatase). Chromatographic detection can also be used.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that can be used for detecting and quantitating biomarker protein levels is Western blotting. Immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Antibodies against biomarkers can also be used for imaging purposes, for example, to detect the presence of a biomarker in cells of a subject. Suitable labels include radioisotopes, iodine (125I, 121I), carbon (14C), sulphur (35S), tritium (3H), indium (112 In), and technetium (99mTc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

For in vivo imaging purposes, antibodies are not detectable, as such, from outside the body, and so must be labeled, or otherwise modified, to permit detection. Markers for this purpose can be any that do not substantially interfere with the antibody binding, but which allow external detection. Suitable markers can include those that can be detected by X-radiography, NMR or MRI. For X-radiographic techniques, suitable markers include any radioisotope that emits detectable radiation but that is not overtly harmful to the subject, such as barium or caesium, for example. Suitable markers for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m.

The labeled antibody or antibody fragment will then preferentially accumulate at the location of cells which contain a biomarker. The labeled antibody or variant thereof, e.g., antibody fragment, can then be detected using known techniques. Antibodies include any antibody, whether natural or synthetic, full length or a fragment thereof, monoclonal or polyclonal, that binds sufficiently strongly and specifically to the biomarker to be detected. An antibody can have a Kd of at most about 10-6M, 10-7M, 10-8M, 10-9M, 10-10M, 10-11M, 10-12M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In certain embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker). In one embodiment, the agent is a labeled or labelable antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. Sec, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample can contain heavy atoms (e.g., 13C) thereby permitting the test sample to be mixed with the known control sample in the same mass spectrometry run.

In certain embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In certain embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to better quantify the amount of the biomolecule present in the first sample. In certain embodiments, the identity of the biomolecules in the first and second sample can also be determined.

### RNA Detection Techniques

In certain embodiments, the biomarker is a nucleic acid, including DNA and/or RNA, contained within the subject or donor organ- or tissue-specific isolated microvesicles, e.g., exosomes. In some embodiments, the biomarker is a miRNA. In certain embodiments, the biomarker is an mRNA. Nucleic acid molecules can be isolated from a microvesicle using any number of methods, which are well-known in the art, the particular isolation procedure chosen being appropriate for the particular biological sample. Examples of methods for extraction are provided in the Examples section herein. In certain instances, with some techniques, it may also be possible to analyze the nucleic acid without extraction from the microvesicle.

In certain embodiments, the analysis of nucleic acids present in the microvesicles is quantitative and/or qualitative. Any method for qualitatively or quantitatively detecting a nucleic acid biomarker can be used. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate RNA levels of biomarker. The levels of a biomarker and a control RNA can be quantitated in cancer tissue or cells and adjacent benign tissues. In certain embodiments, the levels of one or more biomarkers can be quantitated in a biological sample.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization can also be employed. Another method for evaluation of biomarker expression is to detect mRNA levels of a biomarker by fluorescent in situ hybridization (FISH). FISH is a technique that can directly identify a specific region of DNA or RNA in a cell or biological sample and therefore enables to visual determination of the biomarker expression in tissue samples. The FISH method has the advantages of a more objective scoring system and the presence of a built-in internal control including of the biomarker gene signals present in all non-neoplastic cells in the same sample. Fluorescence in situ hybridization is a direct in situ technique that is relatively rapid and sensitive. FISH test also can be automated. Immunohistochemistry can be combined with a FISH method when the expression level of the biomarker is difficult to determine by immunohistochemistry alone.

Alternatively, RNA expression can be detected on a DNA array, chip or a microarray. Oligonucleotides corresponding to the biomarker(s) are immobilized on a chip which is then hybridized with labeled nucleic acids of a test sample obtained from a subject. Positive hybridization signal is obtained with the sample containing biomarker transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor miRNA levels, for example, mRNA can be extracted from the biological sample to be tested, reverse transcribed and fluorescent-labeled cDNA probes are generated. The microarrays capable of hybridizing to a biomarker, cDNA can then probed with the labeled cDNA probes, the slides scanned and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the particular biomarker RNA. The probes can be as short as is required to differentially recognize the particular biomarker RNA transcripts, and can be as short as, for example, 15 bases; however, probes of at least 17 bases, e.g., 18 bases or better 20 bases can be used. In some embodiments, the primers and probes hybridize specifically under stringent conditions to a nucleic acid fragment having the nucleotide sequence corresponding to the target gene. As herein used, the term “stringent conditions” means hybridization will occur only if there is at least 95% and at least 97% identity between the sequences.

The form of labeling of the probes can be any that is appropriate, such as the use of radioisotopes, for example, 32P and 35S. Labeling with radioisotopes can be achieved, whether the probe is synthesized chemically or biologically, by the use of suitably labeled bases.

Exemplary probes and primers that can be used in the methods of the present disclosure are presented below.

### Kits

In certain non-limiting embodiments, the present disclosure provides for a kit for assessing the conditional state of a transplanted organ or tissue in a subject comprising a means (e.g., capturing agent, reagent, technological platform, or combinations thereof) for detecting one or more biomarkers. The disclosure further provides for kits for determining the efficacy of a therapy for preventing or treating organ or tissue rejection in a subject. In certain embodiments, the transplanted tissue can be pancreatic islets. In certain embodiments, the transplanted tissue can be islet beta cells. In certain embodiments, the transplanted tissue can be cultured beta cells.

In certain non-limiting embodiments, the present disclosure provides for a kit for predicting, diagnosing, and/or monitoring diabetes in a subject comprising a means (e.g., capturing agent, reagent, technological platform, or combinations thereof) for detecting one or more biomarkers. The disclosure further provides for kits for determining the efficacy of a therapy for diabetes in a subject. In certain embodiments, the tissue can be pancreatic islets. In certain embodiments, the tissue can be islet beta cells.

Types of kits include, but are not limited to, packaged probe and primer sets (e.g. TaqMan probe/primer sets), arrays/microarrays, biomarker-specific antibodies and beads, which further contain one or more probes, primers or other detection reagents for detecting one or more biomarkers of the present disclosure.

In certain non-limiting embodiments, a kit can comprise a pair of oligonucleotide primers suitable for polymerase chain reaction (PCR) or nucleic acid sequencing, for detecting one or more biomarker(s) to be identified. A pair of primers can comprise nucleotide sequences complementary to a biomarker, and be of sufficient length to selectively hybridize with said biomarker. Alternatively, the complementary nucleotides can selectively hybridize to a specific region in close enough proximity 5′ and/or 3′ to the biomarker position to perform PCR and/or sequencing. Multiple biomarker-specific primers can be included in the kit to simultaneously assay large number of biomarkers. The kit can also comprise one or more polymerases, reverse transcriptase and nucleotide bases, wherein the nucleotide bases can be further detectably labeled.

In certain non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In certain non-limiting embodiments, the oligonucleotide primers can be immobilized on a solid surface or support, for example, on a nucleic acid microarray, wherein the position of each oligonucleotide primer bound to the solid surface or support is known and identifiable.

In certain non-limiting embodiments, a kit can comprise at least one nucleic acid probe, suitable for in situ hybridization or fluorescent in situ hybridization, for detecting the biomarker(s) to be identified. Such kits will generally comprise one or more oligonucleotide probes that have specificity for various biomarkers.

In certain non-limiting embodiments, a kit can comprise at least one antibody for immunodetection of the biomarker(s) to be identified. Antibodies, both polyclonal and monoclonal, specific for a biomarker, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 1311) or enzymes (alkaline phosphatase, horseradish peroxidase).

In certain non-limiting embodiments, the biomarker-specific antibody can be provided bound to a solid support, such as a column matrix, an array, or well of a microtiter plate. Alternatively, the support can be provided as a separate element of the kit.

In certain non-limiting embodiments, a kit can comprise one or more primers, probes, microarrays, or antibodies suitable for detecting one or more biomarkers.

In certain non-limiting embodiments, where the measurement means in the kit employs an array, the set of biomarkers set forth above can constitute at least 10 percent or at least 20 percent or at least 30 percent or at least 40 percent or at least 50 percent or at least 60 percent or at least 70 percent or at least 80 percent of the species of markers represented on the microarray.

In certain non-limiting embodiments, a biomarker detection kit can comprise one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction to detect a biomarker. A kit can also include additional components or reagents necessary for the detection of a biomarker, such as secondary antibodies for use in immunohistochemistry. A kit can further include one or more other biomarkers or reagents for evaluating other prognostic factors, e.g., stage of rejection.

In certain non-limiting embodiments, a biomarker detection kit can comprise one or more reagents and/or tools for isolating donor organ- or tissue-specific microvesicles from a biological sample. In certain non-limiting embodiments, a biomarker detection kit can comprise one or more reagents and/or tools for isolating subject organ- or tissue-specific microvesicles from a biological sample. A kit can also include reagents necessary for isolating the protein and/or nucleic acids from the isolated microvesicles.

A kit can further contain means for comparing the biomarker with a reference standard, and can include instructions for using the kit to detect the biomarker of interest. In certain embodiments, the instructions describes that the change in the level and/or presence of a biomarker, set forth herein, is indicative that the transplanted organ or tissue in a subject is being rejected and/or is injured. In certain embodiments, the instructions describes that the change in the level and/or presence of a biomarker, set forth herein, is indicative that the subject is developing or had diabetes.

### Reports, Programmed Computers, and Systems

The results of a test (e.g., the diabetic state or the conditional state of a transplanted organ or tissue in a subject), or an individual's predicted drug responsiveness (e.g., response to diabetes or immunosuppresion therapy), based on assaying one or more biomarkers, and/or any other information pertaining to a test, can be referred to herein as a “report”. A tangible report can optionally be generated as part of a testing process (which can be interchangeably referred to herein as “reporting,” or as “providing” a report, “producing” a report, or “generating” a report).

Examples of tangible reports can include, but are not limited to, reports in paper (such as computer-generated printouts of test results) or equivalent formats and reports stored on computer readable medium (such as a CD, USB flash drive or other removable storage device, computer hard drive, or computer network server, etc.). Reports, particularly those stored on computer readable medium, can be part of a database, which can optionally be accessible via the internet (such as a database of patient records or genetic information stored on a computer network server, which can be a “secure database” that has security features that limit access to the report, such as to allow only the patient and the patient's medical practitioners to view the report while preventing other unauthorized individuals from viewing the report, for example). In addition to, or as an alternative to, generating a tangible report, reports can also be displayed on a computer screen (or the display of another electronic device or instrument).

A report can include, for example, an individual's medical history, or can just include size, presence, absence or levels of one or more biomarkers (for example, a report on computer readable medium such as a network server can include hyperlink(s) to one or more journal publications or websites that describe the medical/biological implications, such as increased or decreased disease risk, for individuals having certain biomarkers or levels of certain biomarkers). Thus, for example, the report can include risk or other medical/biological significance (e.g., drug responsiveness, suggested prophylactic treatment, etc.) as well as optionally also including the biomarker information, or the report can just include biomarker information without including disease risk or other medical/biological significance (such that an individual viewing the report can use the biomarker information to determine the associated disease risk or other medical/biological significance from a source outside of the report itself, such as from a medical practitioner, publication, website, etc., which can optionally be linked to the report such as by a hyperlink).

A report can further be “transmitted” or “communicated” (these terms can be used herein interchangeably), such as to the individual who was tested, a medical practitioner (e.g., a doctor, nurse, clinical laboratory practitioner, genetic counselor, etc.), a healthcare organization, a clinical laboratory, and/or any other party or requester intended to view or possess the report. The act of “transmitting” or “communicating” a report can be by any means known in the art, based on the format of the report. Furthermore, “transmitting” or “communicating” a report can include delivering a report (“pushing”) and/or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the internet, by facsimile, and/or by any wired or wireless communication methods known in the art) such as by being retrieved from a database stored on a computer network server, etc.

In certain exemplary embodiments, the disclosed subject matter provides computers (or other apparatus/devices such as biomedical devices or laboratory instrumentation) programmed to carry out the methods described herein. For example, in certain embodiments, the disclosed subject matter provides a computer programmed to receive (i.e., as input) the identity of the one or more biomarkers disclosed herein, alone or in combination with other biomarkers, and provide (i.e., as output) the risk (e.g., risk of organ or tissue rejection) or other result (e.g., organ or tissue rejection diagnosis or prognosis, drug responsiveness, etc.) based on the level or identity of the biomarker(s). Such output (e.g., communication of risk, disease diagnosis or prognosis, drug responsiveness, etc.) can be, for example, in the form of a report on computer readable medium, printed in paper form, and/or displayed on a computer screen or other display.

In exemplary embodiments, the system is controlled by the individual and/or their medical practitioner in that the individual and/or their medical practitioner requests the test, receives the test results back, and (optionally) acts on the test results to reduce the individual's disease risk, such as by implementing a disease management system.

The following examples are offered to more fully illustrate the disclosure, but are not to be construed as limiting the scope thereof.

### Example 1: Xenoislet Transplantation Mouse Model

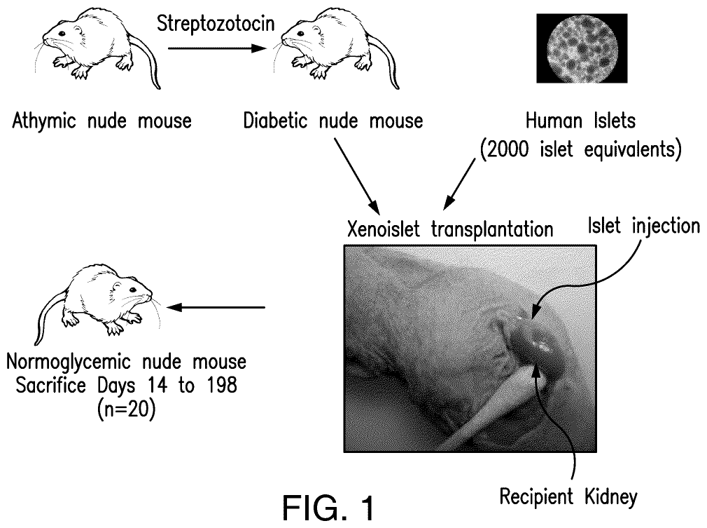

One week prior to xenoislet transplantation, athymic nude mice were made diabetic through an intraperitoneal injection of streptozotocin (200 μg/kg). After confirmation of hyperglycemia (blood glucose >400 mg/dL) for at least 3 days, a critical mass (2000 islet equivalents) of cultured human islets were transplanted, under general anesthesia (2-5% isoflurane, Primal Healthcare Ltd, India), under the recipient renal capsule through a paramedian abdominal incision (FIG. 1). Islet transplantation was performed at the University of Pennsylvania procedural protocols (CIT07 and CIT06). Islet viability, quantity, and function were analyzed.

Human pancreas was processed for islet isolation, and high purity (>80%) islets were used for xenoislet transplantation. Islet isolation was performed by the Islet Core Facility at the University of Pennsylvania in accordance to approved Institutional Review Board protocols. Islets were cultured in CMRL media supplemented with albumin, without any exogenous EV contamination. Islet culture supernatant (20 ml) was obtained 24 to 48 hours post-isolation and EVs were isolated from the supernatant to serve as positive controls for experiments analyzing EV protein and RNA cargoes. Exosomes were isolated from human islet culture supernatants by high exclusion limit agarose-based gel chromatography along with ultracentrifugation. Briefly, 10 ml culture supernatant was centrifuged at 500 g for 10 min to eliminate cell debris, and filtered through a 0.22 μm filter. The filtrate was then passed through a Sepharose 2B column and the eluent was collected in 1 ml fractions. The exosome fraction was pooled after monitoring absorbance at 280 nm. The pooled fraction was ultracentrifuged at 120,000 g for 2 hours at 4° C. The pelleted exosome fraction was resuspended in PBS for downstream analysis. Mouse and human plasma exosome isolation was performed utilizing 200 μl to 1 ml plasma obtained after centrifugation of the blood sample at 500 g for 10 minutes. Plasma sample was directly added to the Sepharose 2B column for exosome isolation as described above.

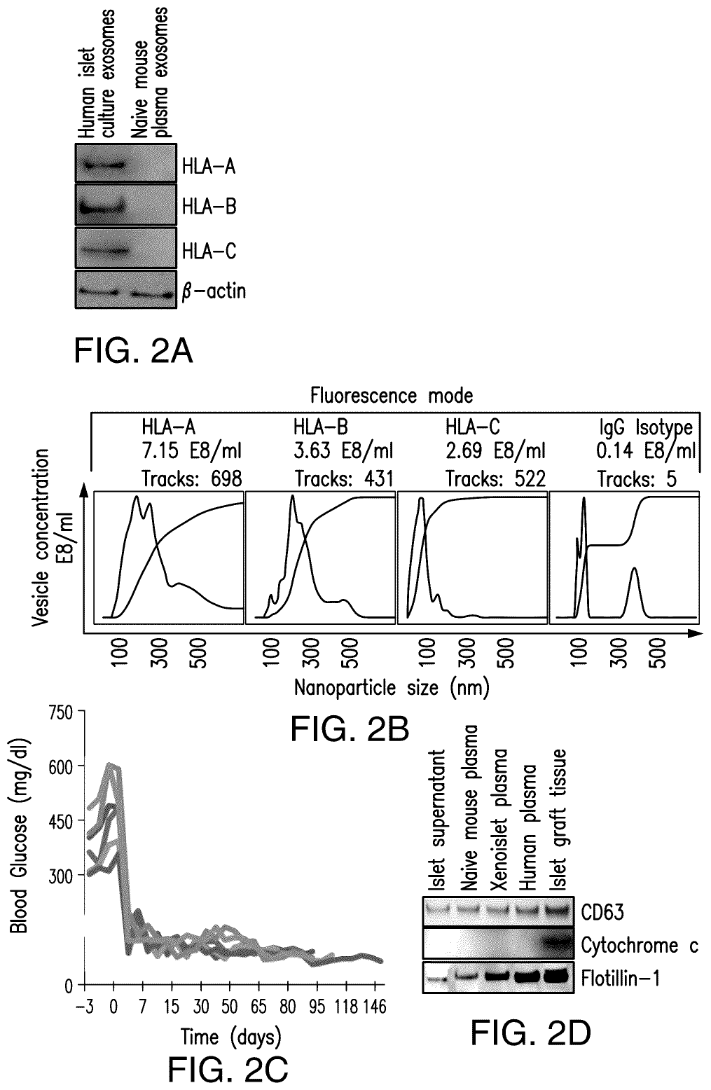

The exosomes released into the supernatant medium were confirmed to express human specific MHC claim I (human leukocyte antigen, HLA) antigens on their surface, which is not detected on naive mouse plasma exosomes (FIG. 2A). FIG. 2B provides NanoSight fluorescence images of exosomes from human pancreatic islet culture for human MHC molecules, HLA-A, HLA-B, and HLA-C. IgG isotype control is also shown. Islet exosome expressed all three HLA class I molecules on their surface.

Recipient animal's glycemic status was monitored for glucose regulation up to 150 days post-transplant at least 3 times a week to ensure normoglycemia (FIG. 2C). FIG. 2C provides representative blood glucose curves for 6 diabetic recipient animals after islet transplantation showed normoglycemia. Extracellular microvesicles isolated using the described methodology were analyzed on Western blot for presence of exosome markers CD63 and flotillin-1, and for the absence of apoptotic body marker cytochrome c (FIG. 2D). The isolated samples showed enrichment of exosomes, without contamination from cellular particles/apoptotic bodies.

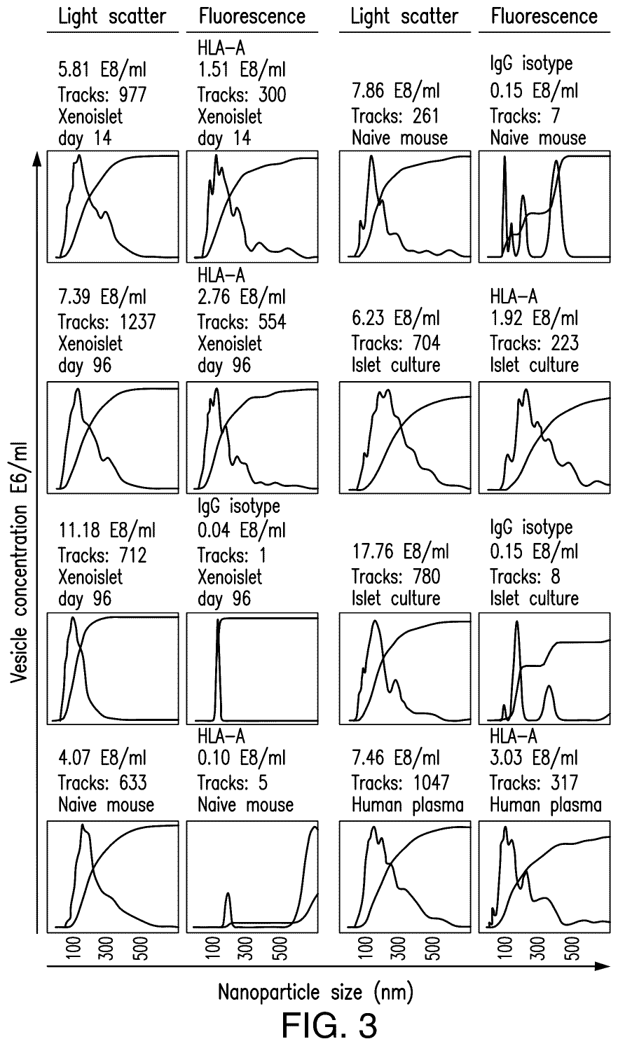

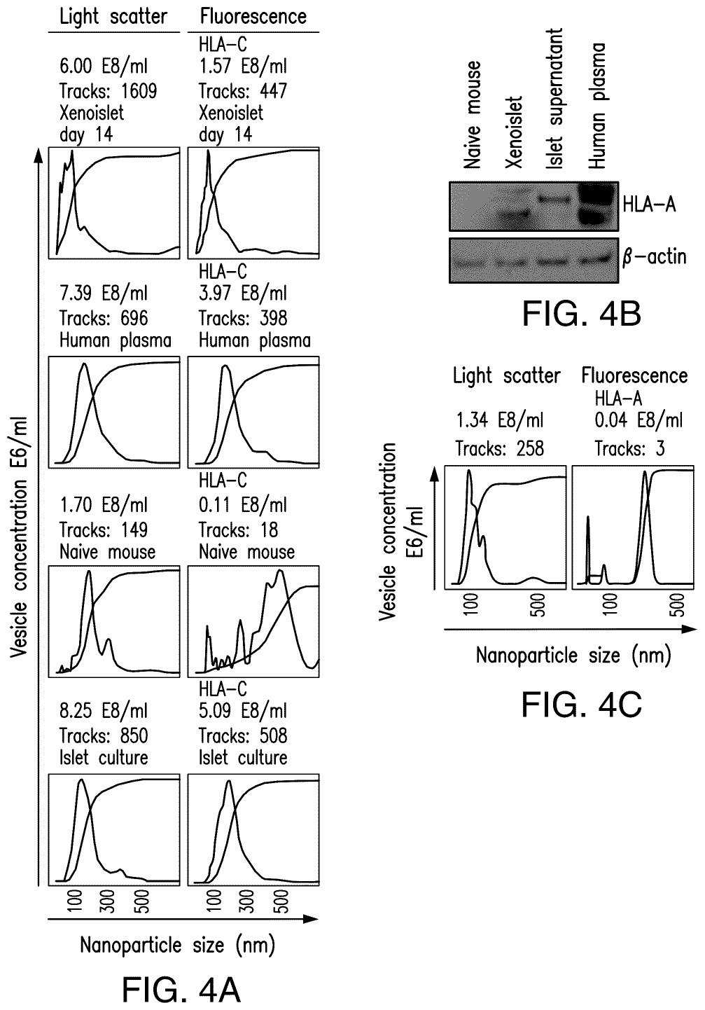

After transplant, the exosomes were analyzed on the NanoSight nanoparticle detector on the fluorescence mode for HLA positive exosomes (range 14 to 150 days; n=25). Light scatter represents total exosome pool per sample, and fluorescence mode represents the exosome subpopulation positive for the tested surface exosome marker using the antibody quantum dot. Representative samples from xenoislet post-transplant days 14 and 96 showed HLA-A and HLA-C expressing exosomes in recipient plasma, compared to IgG isotype controls. Naive mouse (negative control) plasma sample did not show HLA expressing exosome subpopulation. Positive controls included exosomes from human plasma and from supernatant of in vitro human islet culture. For example, at all tested time points, HLA specific exosome signal (anti-HLA-A quantum dot) was detected in the plasma of normoglycemic xenoislet recipients (N-xeno); however, this signal was undetectable in naive mouse (n=10) plasma exosomes (p<0.001) (FIG. 3). Putative TISE signal was also seen using an antibody to another donor tissue specific human MHC marker, HLA-C (FIG. 4A). Further, Western blot analysis confirmed the NanoSight fluorescence findings, as the xenoislet plasma exosomes showed presence of HLA (Figure. 4B).

The specificity of the plasma TISE was examined by performing transplant islet graftectomy (n=6), where the recipient left kidney containing the subcapsular human islets was removed en bloc upon ligation of renal vessels and ureter. The animal was monitored posteoperatively to confirm diabetic status (glucose >400 mg/dL). Islet graftectomy led to severe hyperglycemia in the recipients, and on NanoSight fluorescence, there was complete loss of the donor HLA-A signal post-islet graftectomy (p<0.001; FIG. 4C). FIG. 4C is a representative image from 1 out of 6 animals. Taken together, these data demonstrate that transplanted human islets release donor MHC specific exosomes into recipient plasma, and the TISE signal is specific to the transplanted human islet mass.

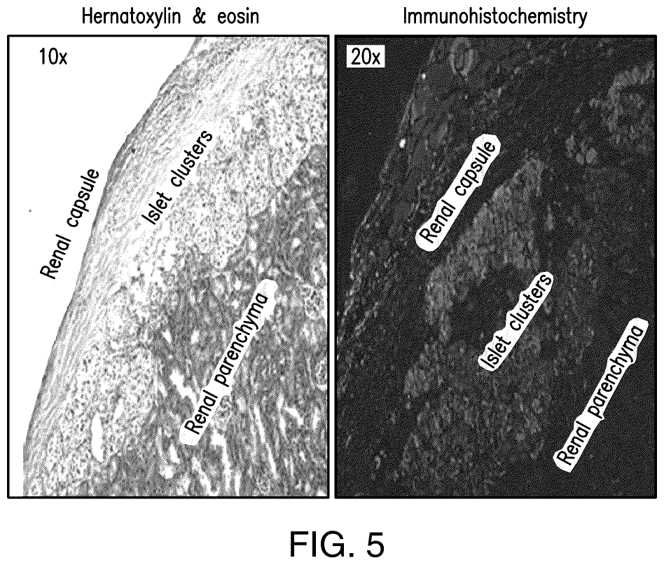

Islet graft tissue was cut with cryostat and fixed with 4% paraformaldehyde after washing with PBS. Blocking solution (0.05% Triton X-100) was added and then tissue slides were treated with primary antibody (insulin (SantaCruz), glucagon (Santa Cruz)) overnight. Slides were washed 3×PBS and the secondary antibody was added for detection. Analysis was performed using Zeiss epifluorescence microscope. Hematoxylin and cosin histology staining of transplanted human islet mass under the mouse renal capsule showed islet clusters without leukocytic infiltration (FIG. 5). Immunohistochemistry for insulin (red) and glucagon (green) confirmed the presence of viable islet clusters (FIG. 5).

### Example 2: Enrichment of Human Transplant Islet Specific Exosomes

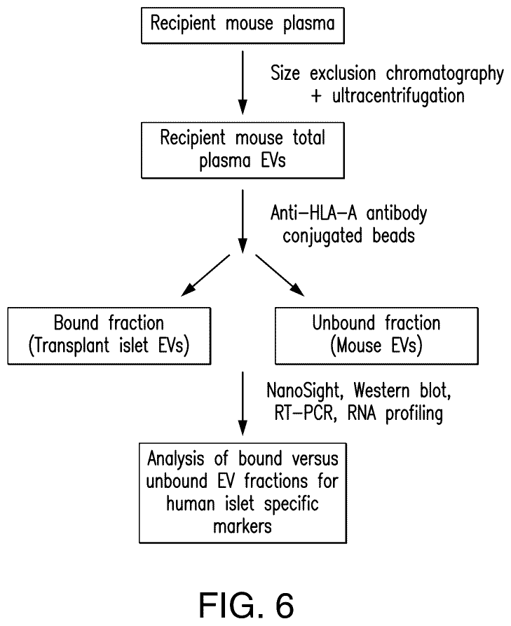

Studies were conducted to determine whether islet specific exosomes could be harvested and enriched to enhance the signal to noise ratio of experimental assays. This characterization would improve accuracy and time sensitivity of the biomarker platform. A schematic of the method of enriching human transplant islet specific exosomes (TISE) obtained from recipient mouse plasma EV pool using anti-HLA specific affinity antibody bead technology is shown in FIG. 6.

### Methods

EVs were isolated from islet culture supernatants by high exclusion limit agarose-based gel chromatography along with ultracentrifugation. 10 ml culture supernatant was centrifuged at 500 g for 10 min to eliminate cells and debris, and filtered through a 0.22 μm filter. The filtrate was then passed through a Sepharose 2B column and the eluent was collected in 1 ml fractions. The EV fraction was pooled after monitoring absorbance at 280 nm. The pooled fraction was ultracentrifuged at 110,000 g for 2 hours at 4° C., and the pelleted EV fraction was resuspended in PBS for downstream analysis. Mouse and human plasma EV isolation was performed utilizing 500 μl to 1 ml plasma obtained after centrifugation of the blood sample at 500 g for 10 minutes. Plasma samples were directly added to the column for EV isolation.

MHC specific antibody was covalently conjugated to N-hydroxysuccinamide magnetic beads (Pierce) per manufacturer's protocol. 50 to 100 ug protein equivalent of EVs were incubated with antibody beads overnight at 4° C. The bead bound and unbound EV fractions were separated per manufacturer's protocol. EVs bound to beads were eluted using tris glycine and utilized for downstream analysis.

Purified EVs were analyzed on the NanoSight NS300 (405 nm laser diode) on the light scatter mode for EVquantification and scatter distribution according to manufacturer's protocols (Malvern instruments Inc., MA, USA). Before each experimental run, the machine was calibrated for nanoparticle size and quantity using standardized nanoparticle and dilutions provided by the manufacturer. Surface marker detection on EVs was performed using the fluorescence mode on the NanoSight NS300. Secondary antibodies conjugated to quantum dots with emission at 605 nm were utilized for fluorescence detection of primary antibodies binding against specific surface proteins as described previously (Gardiner, C. et al., J Extracell Vesicles. 2, 19671 (2013); Dragovic, R. A. et al., Nanomedicine. 7(6), 780-788 (2011)). Each experimental run was performed in duplicates, and an appropriate IgG isotype control fluorescence was performed to assess background.

EV and cell lysate total proteins were isolated and separated on polyacrylamide gels, transferred on polyvinylidene difluoride membrane (Life Technologies, NY, USA). The blot was blocked, incubated with desired antibody at concentration per manufacturer's protocol. Horseradish peroxidase coupled secondary antibody (Santa Cruz Biotechnologies Inc.) was added per manufacturer's protocol and detected through chemiluminescence using Image quant LAS 400 Phospho-Imager (GE Health, USA).

For total protein isolation, EV pellet was lysed in 1×RIPA buffer with 1× concentration of protease inhibitor cocktail (Sigma-Aldrich Co., MO).

Anti-HLA-A, and -B antibodies (Santa Cruz Biotechnologies, Inc. TX, USA) were utilized for NanoSight fluorescent staining and analysis of human islet EVs purified from islet cultures and for recipient mouse plasma analysis. Antibodies to human FXYD2 (Abnova), insulin, glucagon, and somatostatin, CD3, CD4, CD8, CD56, CD19, CD56, TSG101, aquaporin 2, podocalyxin-1, and to mouse MHC I were purchased from Santa Cruz Biotechnologies, Inc. Secondary antibodies and isotype controls (anti-goat, anti-rabbit, anti-mouse, goat IgG, rabbit IgG, and mouse IgG) were also purchased from Santa Cruz Biotechnologies, Inc. Anti-goat, anti-rabbit, and anti-mouse conjugated quantum dot (605 nm) were purchased from Life Technologies (Grand Island, NY, USA) and utilized per manufacturer's protocol for NanoSight fluorescent analysis.

Exosomes suspended in PBS were processed at the Electron Microscopy Resource Laboratory, University of Pennsylvania, using the standard protocols. Briefly, 50 ul of exosomes were absorbed onto forvar carbon coated nickel grid for 1 hour. Then the grids were sequentially washed with 0.1M sodium cacodylate, PH 7.6 and fixed in 2% paraformaldehyde and 2.5% glutaraldehyde in 0.1M sodium cacodylate, contrasted with 2% uranyl acetate in 0.1M sodium cacodylate for 15 min. After another washing, grids were incubated with 0.13% methyl cellulose and negatively stained with 0.4% uranyl acetate for 10 min, air dried and visualized under the JEM-2200FS transmission electron microscope operated at 100 kV.

### Results

TISE were enriched from N-xeno plasma exosomes using anti-HLA-A specific affinity antibody beads to obtain an HLA-A bound fraction representing TISE, and HLA-A unbound fraction representing the non-transplant tissue specific recipient exosomes.

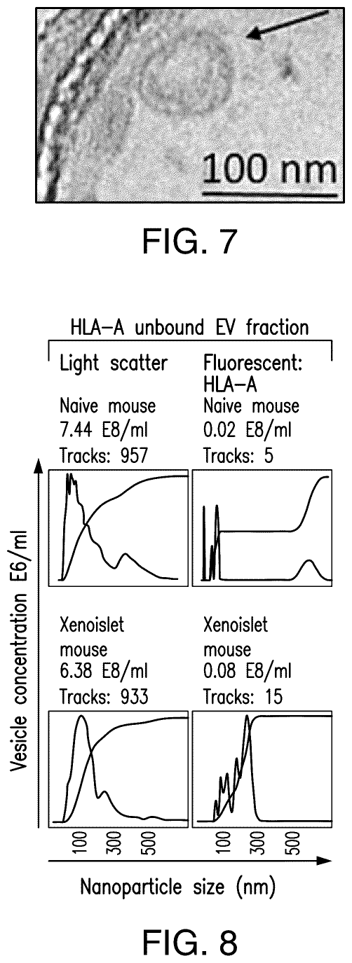

To validate that the antibody beads are binding intact TISE and not freely circulating plasma protein aggregates, transmission electron microscopy of eluted HLA-A bound exosomes was preformed. Distinct, intact exosomes (40 nm to 100 nm range) were noted on electron microscopy (FIG. 7).

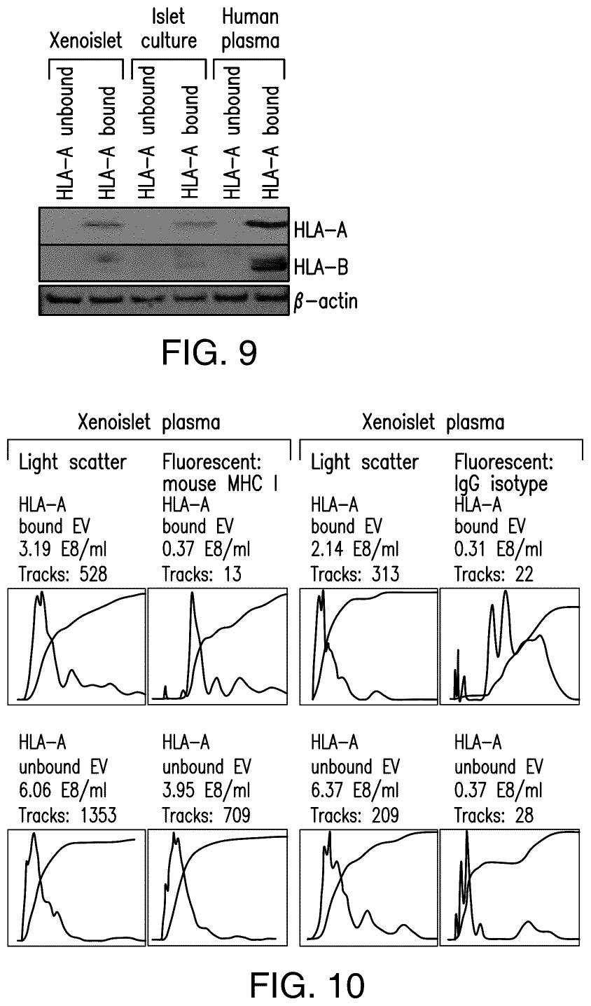

To validate optimal capture of the donor tissue EVs, it was confirmed that HLA-A unbound EV fraction was HLA negative (FIG. 8). The unbound fractions in both samples failed to show HLA-A signal, signifying that HLA-A positive EVs from xenoislet plasma were adequately enriching, with minimal, if any, HLA-A positive EVs left in the unbound fraction. It was also confirmed that HLA-A bound EV fraction was HLA-A positive, and that it contained minimal mouse EV contamination (FIGS. 9 and 10). In particular, the HLA-A bound and unbound EV fractions were assessed for expression of HLA-A and HLA-B on Western blot analysis (FIG. 9). In the xenoislet, human islet culture supernatant, and human plasma samples, the HLA-A bound fractions showed expression of HLA-A and HLA-B proteins; but not in the HLA-A unbound fractions. This shows that the anti-HLA-A antibody beads were very sensitive for binding HLA-A positive EVs. The HLA-A bound and unbound EV fractions from the xenoislet plasma sample were also analyzed for mouse MHC class I positivity on NanoSight using anti-mouse specific MHC I antibody-quantum dot (FIG. 10). The majority of the HLA-A unbound EVs showed mouse MHC I expression, but the HLA-A bound EV fraction did not show mouse MHC I signal above background. This indicated that the HLA-A bound EV fraction in the xenoislet sample had minimal contamination from mouse MHC I positive EVs.

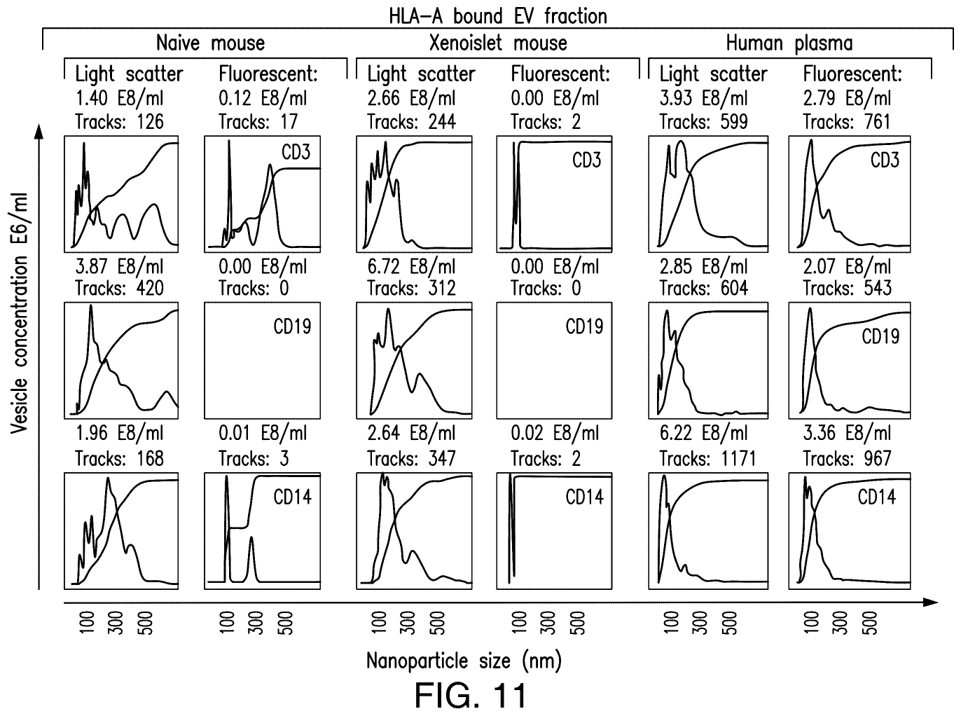

To assess if the HLA-A positive EV signal in recipient xenoislet mouse plasma was due to the release of EVs into mouse blood by donor human passenger leukocytes, the HLA-A bound EV fraction was analyzed on the NanoSight for human specific leukocytes markers using quantum dots conjugated to anti-human CD3 (T cell), anti-human CD19 (B cell), and anti-human CD14 (monocyte). The HLA-A bound EV fraction was negative for human leukocyte markers in xenoislet mouse plasma samples (FIG. 11). Naive mouse plasma EVs served as negative control. Human plasma HLA-A bound EVs served as positive control, as it would be expected for leukocyctes to release HLA class I positive EVs into peripheral circulation. No donor passenger leukocyte EVs in the HLA-A bound EV fraction was found, confirming the absence of human T-cell (CD3), B-cell (CD19), and monocyte (CD14) markers on NanoSight fluorescence. This indicates minimal donor leukocyte exosome contamination.

### Example 3: Analysis of Human Transplant Islet Specific EVs Purified from Recipient Mouse Total Plasma EV Pool in the Xenoislet Model

This study examined the specificity of the putative transplant islet specific exosomes for transplant islet endocrine cellular constituents. The transplant islet specific exosomes were then tested to determine if they carried islet specific endocrine hormones as part of their proteomic and RNA cargoes.

### Methods

In xenoislet animals, to study if the HLA EV signal was specific to the transplanted islet tissue, islet graftectomy was performed post-transplant under general anesthesia by reopening the flank incision. The kidney containing the human islet transplant mass was removed en bloc upon ligation of renal vessels and the ureter. The animal was then monitored postoperatively for 7 days (n=3) and 21 days (n=6). The transplanted islet mass was excised from the mouse renal capsule and RNA and protein were extracted from the transplant mass microarray profiling, and to serve as islet tissue positive control in the Western blot and RT-PCR assays analyzing EV cargo.

For purification of total RNA (including microRNA and mRNA), RNA was extracted from cells and EVs using Trizol, followed by RNeasy mini kit, according to manufacturer's protocol (Qiagen, Germany). For total protein isolation, EV pellet was lysed in 1×RIPA buffer with 1× concentration of protease inhibitor cocktail (Sigma-Aldrich Co., MO).

Total RNA (25 to 50 ng) from islet cells and EVs were reverse transcribed with the SuperScript III one-step RT-PCR system (Life Technologies) for gene expression validation. The primers used in this study are as follows: human insulin (forward) 5′-CCTTGTGAACCAACACCTG-3′, (reverse) 5′-GTAGAAGAAGCCTCGTTCCC-3′(80 bp); human glucagon (forward) 5′-CCCAAGATTTTGTGCAGTGGTT-3′, (reverse) 5′-CAGCATGTCTCTCAAATTCATCGT-3′(80 bp); human somatostatin (forward) 5′-(reverse) 5′-CCGGGTTTGAGTTAGCAGATCT-GATGCCCTGGAACCTGAAGA-3′, 3′(82 bp); human FXYD2γa (forward) 5′-ACTGGGTTGTCGATGGACGGT-3′, (reverse) 5′-CGGCTCATCTTCATTGATTTG-3′(188 bp); and human β-actin primers are (forward) 5′-CTGTACGCCAACACAGTGCT-3′, (reverse) 5′-GCTCAGGAGGAGCAATGATC-3′ (127 bp).

For proteomic analysis of exosomes NHS-HLA-A conjugated beads bound exosomes were eluted in 50 μl volume of tris-glycine (lug protein) and digested with trypsin, analyzed with nano UPLC/MS/MS on the Orbitrap Elite hybrid mass spectrometer (Thermo Scientific) at the Penn Proteomic Core, University of Pennsylvania, Pennsylvania. The data were analyzed with PD/Scaffold software package to search human protein databases, with the following cut-offs: peptide confidence value <95% and protein confidence value <99%.

The xenoislet mouse model is development by the same methods as described in Example 1, and EV enrichment, Western blot, and NanoSight methods are the same as above in Example 2.

### Results

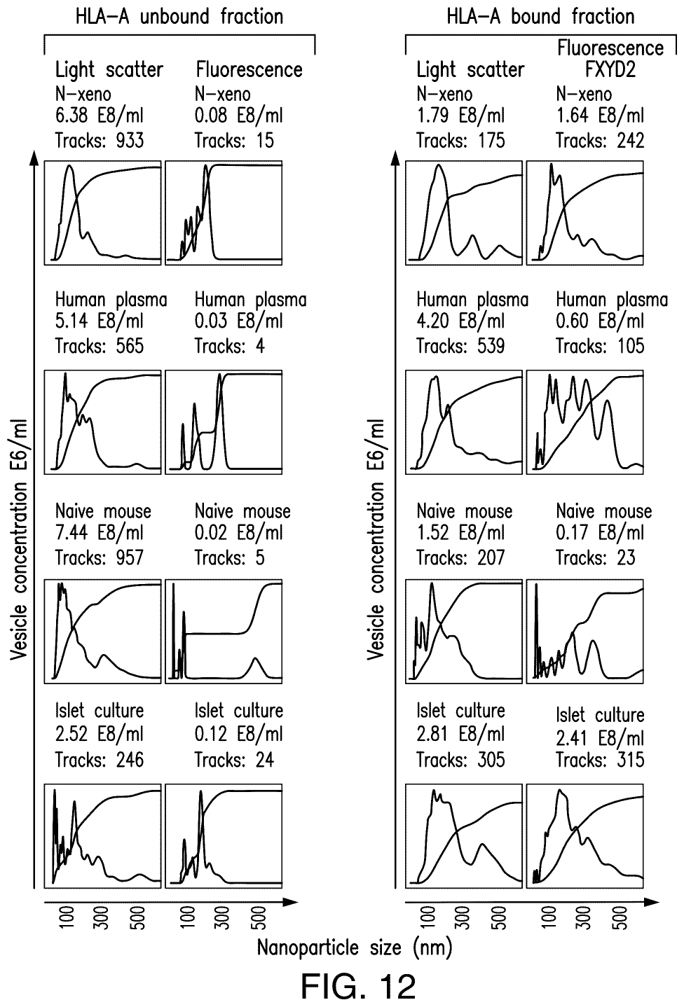

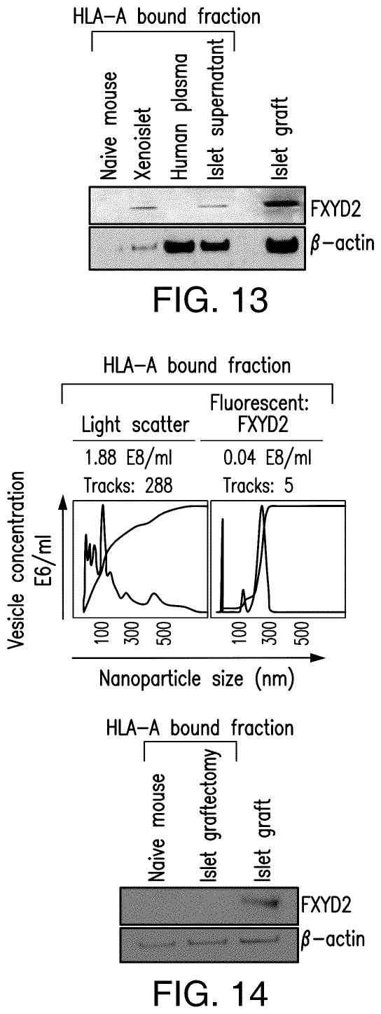

The transmembrane protein ion channel regulator, FXYD2 isoforms ya and yb, is reported to be an islet specific surface marker compared to exocrine pancreas. Therefore, FXYD2 surface expression was tested in HLA-A bound EV fraction. On the NanoSight, xenoislet and human islet culture supernatant EV samples showed the presence FXYD2, but FXYD2 was not present in human and naive mouse plasma samples (FIG. 12). Naive human plasma was analyzed to confirm that FXYD2 surface co-expression was specific to the human islet exosomes, not all HLA-A expressing human plasma exosomes. These finding were confirmed by Western blot (FIG. 13). Furthermore, in xenoislet recipients undergoing islet graftectomy (i.e., removal of the transplanted human cultured islets), the HLA-A bound EV fraction did not show FXYD2 expression via NanoSight or Western blot (FIG. 14).

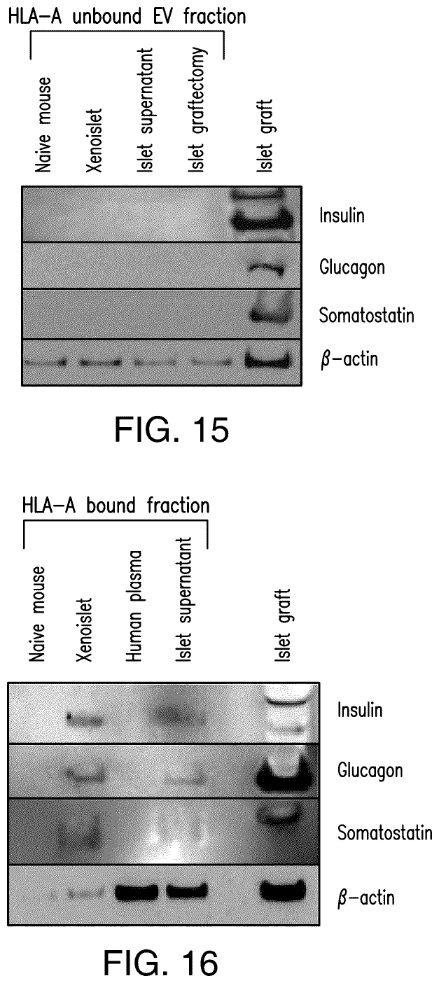

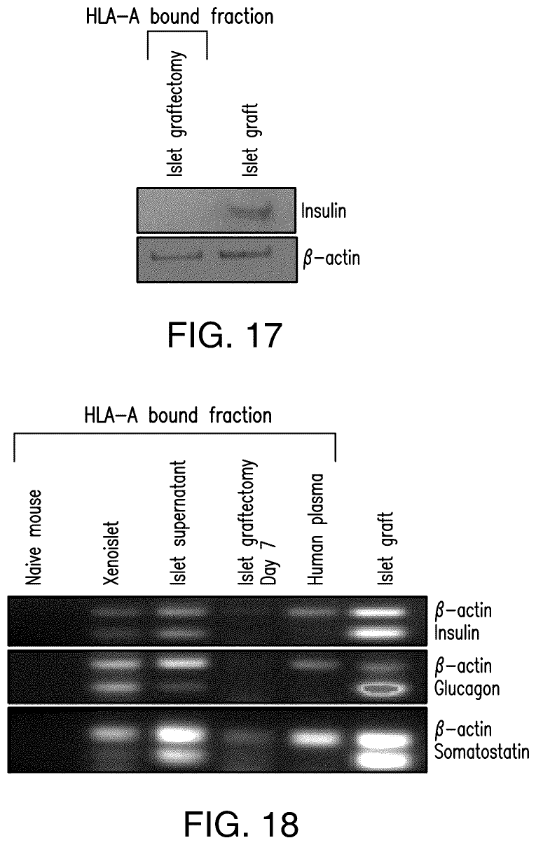

HLA-A bound EV fractions were analyzed for the presence of islet endocrine hormones. Unlike HLA-A unbound fractions (FIG. 15), HLA-A bound EVs from xenoislet and islet culture supernatant samples showed expression of insulin, glucagon, and somatostatin (FIG. 16). In particular, Western blot analysis showed expression of islet endocrine hormones insulin, glucagon, and somatostatin in xenoislet and human islet culture supernatant samples but not in naive mouse and human plasma samples (FIG. 16). A human plasma sample was utilized to assess the possibility that freely circulating insulin, glucagon, and somatostatin were non-specifically binding to the HLA-A beads. If this were the case, then the HLA-A bound EV fraction in the human plasma sample would be positive for endocrine hormone proteins, like the xenoislet and islet supernatant samples. Naive mouse HLA-A bound EV fraction analysis showed that there was a lack of non-specific binding to the HLA-A beads by the freely circulating endogenous mouse islet endocrine hormones. Moreover, the HLA-A bound EV fraction from post-islet graftectomy samples failed to show insulin presence on Western blot (FIG. 17), consistent with the FXYD2 findings. These results provided further validation that TISE were being purified, as only islet ß cell produce insulin.

The presence of islet endocrine hormone mRNAs in the HLA-A bound EV fractions in both the xenoislet animals and post-islet graftectomy animals was assessed using RT-PCR. RNA cargo analysis of HLA-A bound EVs showed islet endocrine hormones, insulin, glucagon, and somatostatin, mRNAs only in the xenoislet sample, but not in naive mouse and human plasma (FIG. 18). The fact that human plasma HLA-A bound fraction failed to show enrichment of endocrine hormone mRNA signals, indicates that the HLA-A bound EV fraction in xenoislet samples was transplant islet specific exosomes, and that the HLA-A bound beads were not non-specifically binding endogenous circulating free plasma insulin mRNA.

Given the HLA signal was absent at 21 days post-graftectomy, HLA-A bound EV fractions were analyzed at 7 days post-graftectomy (n=3). The Islet graftectomy Day 7 sample showed a faint insulin and glucagon mRNA signal, suggesting that transplant islet specific exosomes were not completely cleared out of the recipient circulation at this time point (FIG. 18).

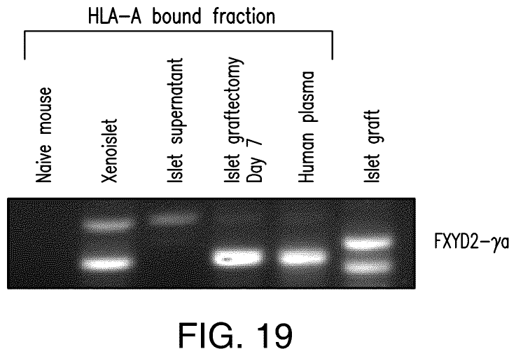

RT-PCR analysis of HLA-A bound EV fractions showed presence of FXYD2γa isoform in xenoislet and islet culture supernatant samples, but not in naive mouse plasma (FIG. 19).

These data demonstrated that islet transplant tissue specific exosomes from recipient plasma were successfully purified using anti-donor MHC specific antibody beads, and that islet exosomes carry islet endocrine hormone mRNA s and proteins as part of their intra-exosomal cargo.

### Example 4: RNA Profiling of Transplant Islet Specific Exosomes

An in vivo read-out of plasma transplant islet specific exosomes mRNA was assessed as compared to its transplanted human islet tissue counterpart (obtained by excision of the islet mass from the recipient renal capsule). In particular, the affymetrix microarray of the long and small RNA cargo of transplant islet specific exosomes and its transplanted human islet tissue counterpart were analyzed for this study. To obtain adequate RNA for profiling, HLA-A bound EVs from three xenoislet animals that received human islets from a single donor were pooled. Histology of the transplanted islet grafts from these animals presented viable islet clusters with no rejection or inflammation.

### Methods

For purification of total RNA (including microRNA and mRNA), RNA was extracted from cells and EVs using Trizol, followed by RNeasy mini kit, according to manufacturer's protocol (Qiagen, Germany).

TISE and cell total RNA representing the transplanted human xenoislet mass was analyzed using the Agilent 2100 Bioanalyzer and Nanodrop spectrophotometry at the University of Pennsylvania Molecular Profiling Facility. All protocols were performed according to the NuGEN Ovation Pico WTA system v2 user guide and the Affymetrix GeneChip Expression Analysis Technical Manual. Briefly, 50 to 100 ng of total RNA was converted to first-strand cDNA using reverse transcriptase primed by poly(T) and random oligomers that incorporated an RNA priming region. Second-strand cDNA synthesis was followed by ribo-SPIA linear amplification of each transcript using an isothermal reaction with RNase, RNA primer and DNA polymerase, and the resulting ssDNA was assessed by Bioanalyzer, fragmented and biotinylated by terminal transferase end labeling. Five and a half micrograms of labeled cDNA were added to Affymetrix hybridization cocktails, heated at 99° C. for 5 min and hybridized for 16 hours at 45° C. to human transcriptome 2.0 ST GeneChips (Affymetrix Inc., Santa Clara CA) using the GeneChip Hybridization oven 645. The microarrays were then washed at low (6×SSPE) and high (100 mM MES, 0.1M NaCl) stringency and stained with streptavidin-phycoerythrin. Fluorescence was amplified by adding biotinylated anti-streptavidin and an additional aliquot of streptavidin-phycoerythrin stain. A GeneChip 3000 7G scanner was used to collect fluorescence signal. Affymetrix Command Console and Expression Console were used to quantitate expression levels for targeted genes; default values provided by Affymetrix were applied to all analysis parameters.

Small RNA microarray analysis was performed at the Molecular Profiling Facility, University of Pennsylvania. Quality control tests of the total RNA samples were done by using the Agilent Bioanalyzer and Nanodrop spectrophotometry. Standard protocols were conducted as described in the Affymetrix FlashTag™ Biotin HSR RNA Labeling Kit manual and the Affymetrix GeneChip Expression Analysis Technical Manual. Briefly, RNA samples were submitted to a tailing reaction followed by ligation of the biotinylated signal molecule to the target RNA sample. Labeled RNA was added to Affymetrix hybridization cocktails, heated at 99° C. for 5 min and hybridized for 16 h at 48° C. to MicroRNA 4.0 GeneChips (Affymetrix Inc., Santa Clara CA) using the GeneChip Hybridization oven 645. The microarrays were then washed at low (6×SSPE) and high (100 mM MES, 0.1M NaCl) stringency and stained with streptavidin-phycoerythrin. Fluorescence was amplified by adding biotinylated anti-streptavidin and an additional aliquot of streptavidin-phycoerythrin stain. A GeneChip 3000 7G scanner was used to collect fluorescence signal. Affymetrix Command Console and Expression Console were used to quantitate expression levels for targeted microRNAs, default values provided by Affymetrix were applied to all analysis parameters.

The islet graftectomy is the same method as described in Example 3.

### Results

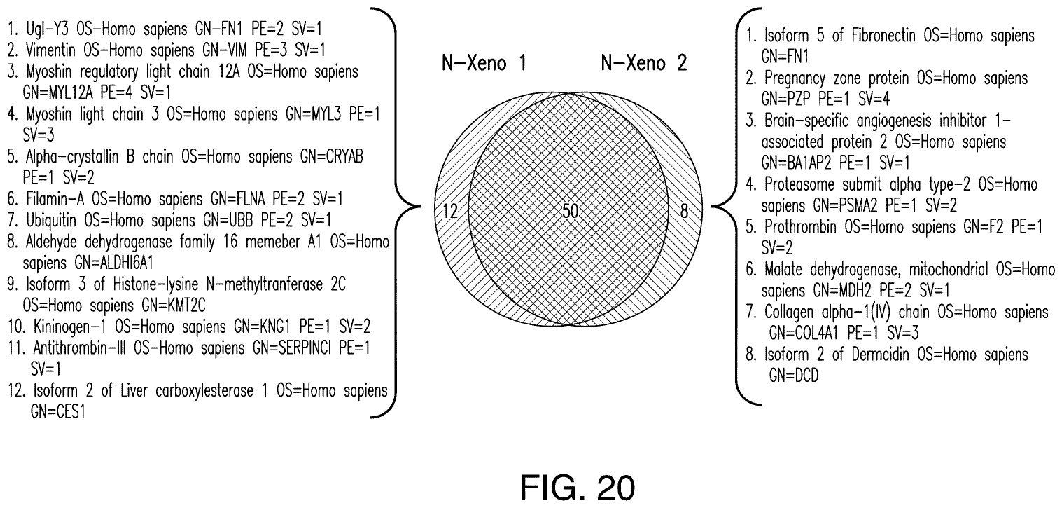

TISE from two independent experiments were analyzed on mass spectrometry for proteomic profiling (FIG. 20). A majority of the proteins were commonly expressed in both TISE fractions. The proteins seen in N-xeno1(12) but not seen in N-xeno2, and vice versa (8 proteins in N-xeno2) are listed. In comparing differences between N-xeno and R-xeno proteomic profiles, only the 50 common proteins in the two N-xeno samples were used, and confirmed that they were human-derived.

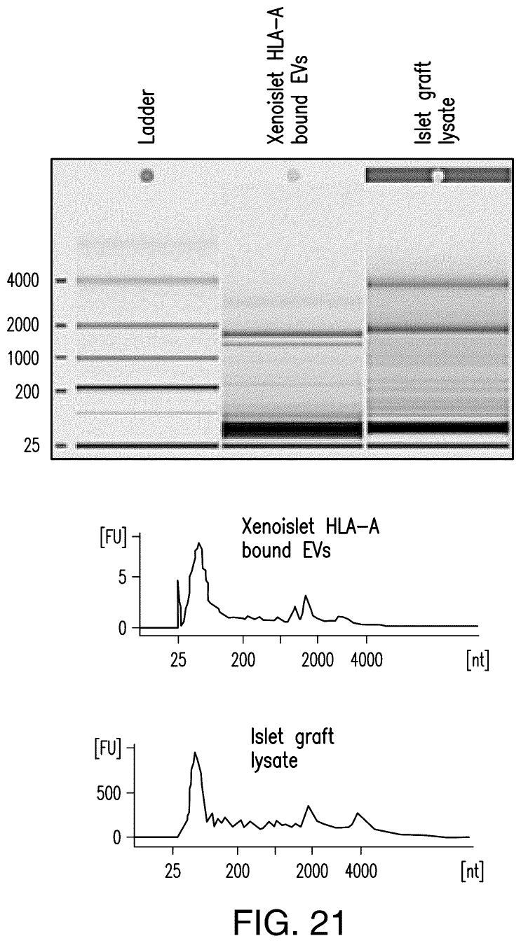

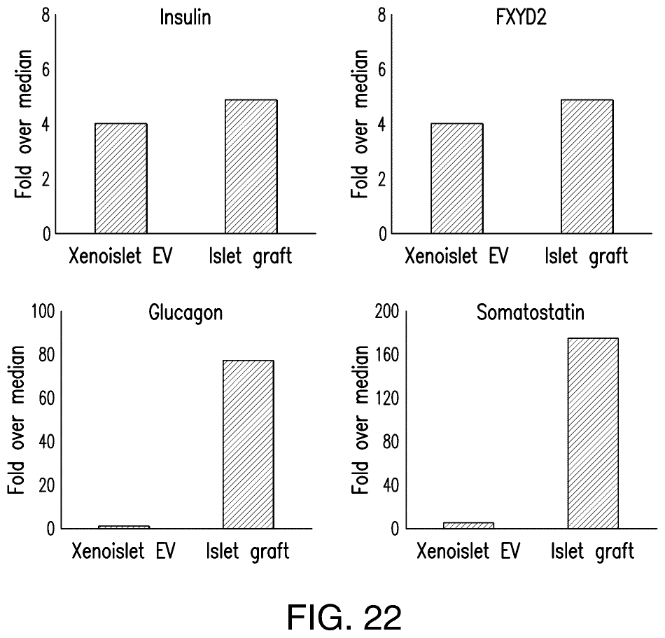

RNA was isolated from TISE and isleft graft tissue of 2 N-xeno animals for long and small RNA microarray profiling using Agilent 2100 Bioanalyzer. Electrophoresis gel of total RNA cargo and bioanalyzer size analysis revealed enrichment of small RNA in the transplant islet specific exosomes compared to the islet tissue graft RNA cargo (FIG. 21). In particular, transplant islet specific exosomes were rich in small RNA (<30 nucleotides), with minimal ribosomal RNA, unlike the transplanted islet tissue. transplant islet specific exosomes microarray further validated mRNA expression of insulin, glucagon, somatostatin, and FXYD2 (FIG. 22). Levels are shown as fold expression over the median value for the microarray set.

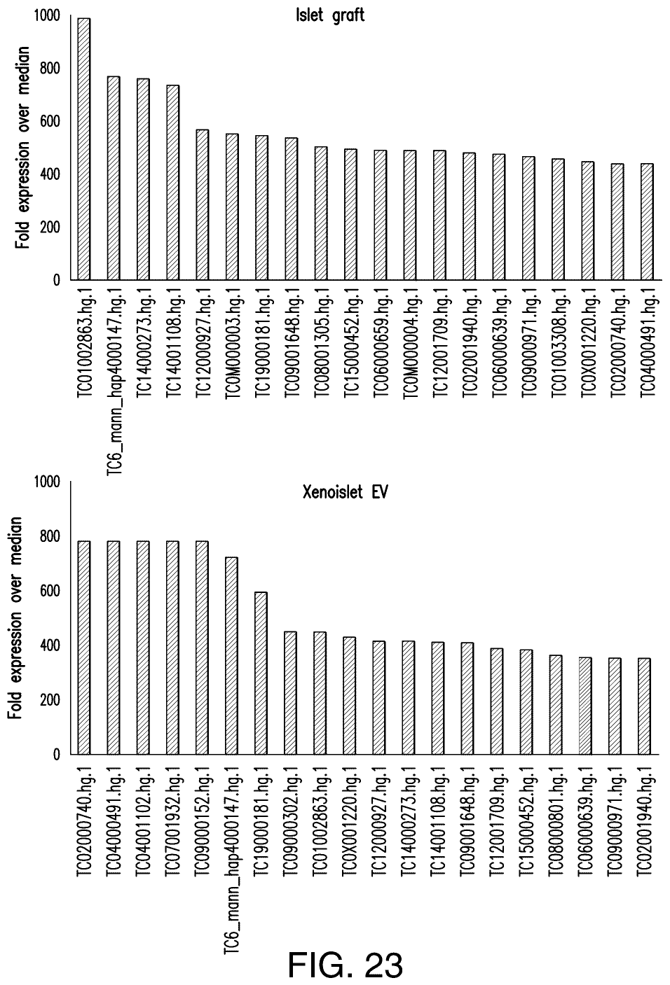

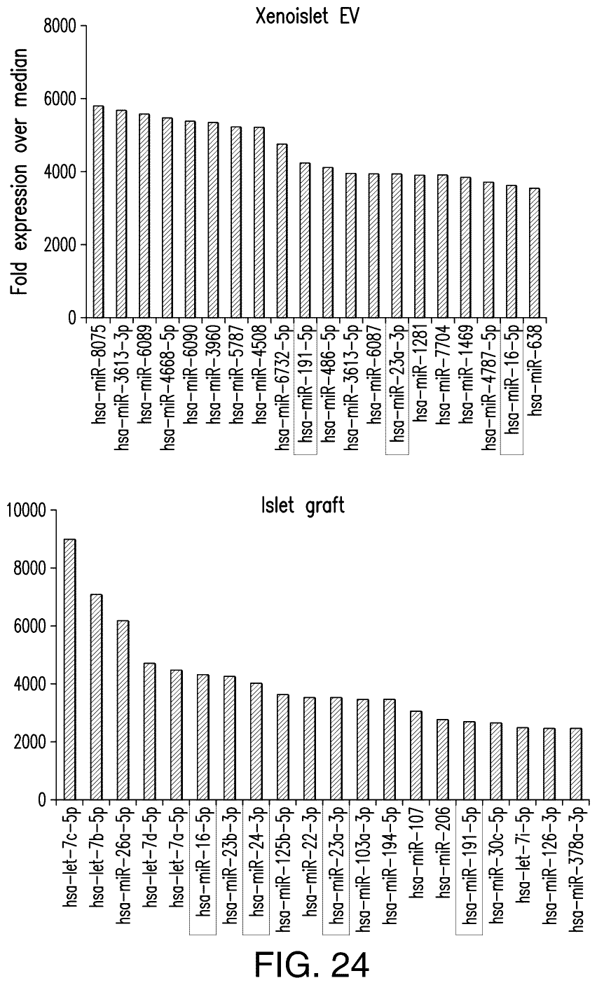

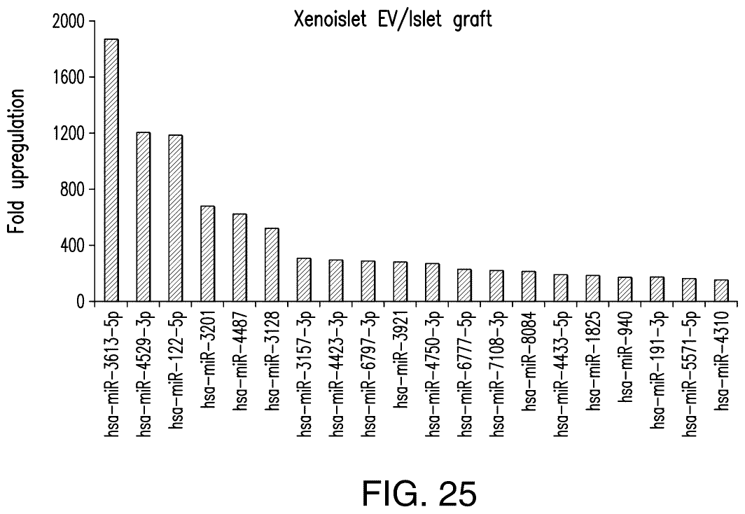

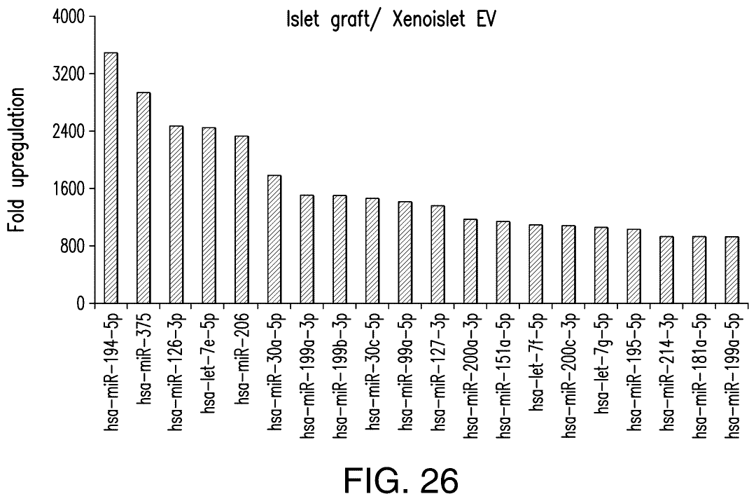

The 25 highest expressing microRNAs in TISE and their relative levels in islet graft, and vice versa are shown in Table 1. The highest expressing microRNAs in TISE were distinct from those expressed in the islet graft. Also, the 25 highest upregulated microRNAs in TISE compared to islet graft (highest enrichment), and vice versa are shown in Table 2. The 20 highest expressing long and microRNAs in transplant islet specific exosomes and islet graft tissue are shown in FIGS. 23 (long RNA) and 24 (microRNA). Unlike long RNA expression, which was similar between the two samples, microRNA profile of transplant islet specific exosomes was different than the excised islet graft tissue with only four microRNAs being common between the two samples: miR-191-5p, miR-23a-3p, miR-16-5p, and miR-24-3p. Interestingly, some microRNAs were highly enriched in transplant islet specific exosomes (FIG. 25) compared to islet tissue (FIG. 26) (e.g., miR-4529-3p). Although most of these microRNAs are not well studied, miR-122, reported to be liver specific, was highly enriched in transplant islet specific exosomes (1191-fold). miR-122 has pro-insulinogenic effects in hepatocytes by upregulating hepatocyte lipid and cholesterol synthesis, and inhibiting gluconeogenesis. Highly expressed ß-cell specific microRNA, miR-375 was markedly down-regulated in transplant islet specific exosomes (2922-fold enriched in islet graft). In particular, miR-3613-5p was the most upregulated microRNA in transplant islet specific exosomes (1843-fold), and miR-3613-5p is predicted to bind targets such as Mbnl2, an RNA binding protein mediating pre-mRNA alternative splicing and expression, including insulin receptor isoforms (www.targetscan.org, www.mirdb.org).

Taken together, these data demonstrate that anti-donor MHC specific exosome purification from recipient plasma enables detailed characterization of the proteomic and RNA signatures of exosomes released by the transplant tissue into recipient circulation.

TABLE 1
Top 25 highest expressing microRNAs in TISE and islet graft tissue from a
normoglycemic xenoislet animal

| TISE | | | Islet Graft | | |
|---|---|---|---|---|---|
| | | TISE | | | Islet graft |
| | Expression | expression/ | | Expression | expression/ |
| | over | Islet graft | | over | TISE |
| microRNA | median | expression | microRNA | median | expression |
| hsa-miR-8075 | 5783.0 | 114.8 | hsa-let-7c-5p | 8985.5 | 2.9 |
| hsa-miR-3613-3p | 5674.6 | 57.8 | hsa-let-7b-5p | 7073.9 | 2.3 |
| hsa-miR-4668-5p | 5452.2 | 66.3 | hsa-miR-26a-5p | 6210.9 | 13.8 |
| hsa-miR-5787 | 5220.3 | 8.7 | hsa-let-7d-5p | 4707.9 | 10.5 |
| hsa-miR-4508 | 5204.4 | 9.3 | hsa-let-7a-5p | 4483.4 | 263.0 |
| hsa-miR-6732-5p | 4776.7 | 70.0 | hsa-miR-16-5p | 4315.4 | 1.2 |
| hsa-miR-486-5p | 4093.8 | 13.9 | hsa-miR-23b-3p | 4258.1 | 5.0 |
| hsa-miR-3613-5p | 3950.7 | 1666.8 | hsa-miR-24-3p | 4011.4 | 1.2 |
| hsa-miR-1281 | 3901.0 | 85.5 | hsa-miR-125b-5p | 3644.2 | 213.8 |
| hsa-miR-7704 | 3898.0 | 6.3 | hsa-miR-22-3p | 3545.8 | 38.7 |
| hsa-miR-1469 | 3836.1 | 5.5 | hsa-miR-23a-3p | 3542.8 | 0.9 |
| hsa-miR-4787-5p | 3717.5 | 4.8 | hsa-miR-103a-3p | 3485.6 | 6.7 |
| hsa-miR-638 | 3547.1 | 6.3 | hsa-miR-194-5p | 3482.2 | 3914.1 |
| hsa-miR-6803-5p | 3520.9 | 4.5 | hsa-miR-107 | 3078.4 | 5.4 |
| hsa-miR-455-3p | 3403.3 | 35.3 | hsa-miR-206 | 2775.6 | 2607.2 |
| hsa-miR-4466 | 3296.9 | 4.3 | hsa-miR-191-5p | 2714.9 | 0.6 |
| hsa-miR-93-5p | 3242.1 | 3.1 | hsa-miR-30c-5p | 2680.9 | 1635.9 |
| hsa-miR-1825 | 3181.1 | 164.2 | hsa-let-7i-5p | 2490.9 | 27.2 |
| hsa-miR-3201 | 3137.5 | 604.0 | hsa-miR-126-3p | 2466.2 | 2772.1 |
| hsa-miR-1237-5p | 3053.4 | 7.2 | hsa-miR-378a-3p | 2464.5 | 561.0 |
| hsa-miR-3656 | 3049.0 | 4.3 | hsa-miR-145-5p | 2406.3 | 13.7 |
| hsa-miR-4529-3p | 3031.3 | 1073.2 | hsa-miR-375 | 2354.5 | 3284.9 |
| hsa-miR-762 | 2977.1 | 9.8 | hsa-miR-192-5p | 2304.1 | 268.8 |
| hsa-miR-17-5p | 2948.6 | 3.4 | hsa-miR-29a-3p | 2207.8 | 5.6 |
| hsa-miR-8069 | 2724.9 | 4.6 | hsa-let-7e-5p | 2102.9 | 2752.0 |

Expression value for microRNA was normalized to the median value for that microarray. The relative expression in TISE compared to Islet graft (TISE/Islet graft) and vice versa is also shown.

TABLE 2
Specific microRNAs are upregulated and down-regulated
in TISE compared to the transplant islet graft.

| | Fold | | Fold down- |
|---|---|---|---|
| | upregulation | | regulation |
| | in TISE | | in TISE |
| | (TISE/ | | (Islet graft/ |
| miRNA | Islet graft) | miRNA | TISE) |
| hsa-miR-3613-5p | 1666.8 | hsa-miR-194-5p | −3914 |
| hsa-miR-4529-3p | 1073.2 | hsa-miR-375 | −3285 |
| hsa-miR-122-5p | 1059.7 | hsa-miR-126-3p | −2772 |
| hsa-miR-3201 | 604.0 | hsa-let-7e-5p | −2752 |
| hsa-miR-4487 | 552.4 | hsa-miR-206 | −2607 |
| hsa-miR-3128 | 463.8 | hsa-miR-30a-5p | −2004 |
| hsa-miR-3157-3p | 270.4 | hsa-miR-199a-3p | −1693 |
| hsa-miR-4423-3p | 265.8 | hsa-miR-199b-3p | −1693 |
| hsa-miR-6797-3p | 252.9 | hsa-miR-30c-5p | −1636 |
| hsa-miR-3921 | 248.2 | hsa-miR-99a-5p | −1584 |
| hsa-miR-4750-3p | 239.7 | hsa-miR-127-3p | −1526 |
| hsa-miR-6777-5p | 202.4 | hsa-miR-200a-3p | −1313 |
| hsa-miR-7108-3p | 192.4 | hsa-miR-151a-5p | −1274 |
| hsa-miR-8084 | 188.4 | hsa-let-7f-5p | −1231 |
| hsa-mir-6776 | 179.0 | hsa-miR-200c-3p | −1213 |
| hsa-miR-4433-5p | 168.3 | hsa-let-7g-5p | −1187 |
| hsa-miR-1825 | 164.2 | hsa-miR-195-5p | −1164 |
| hsa-mir-520g | 156.7 | hsa-miR-214-3p | −1058 |
| hsa-mir-520h | 156.7 | hsa-miR-181a-5p | −1055 |
| hsa-miR-940 | 156.1 | hsa-miR-199a-5p | −1041 |
| hsa-miR-191-3p | 155.9 | hsa-miR-652-3p | −1002 |
| hsa-miR-5571-5p | 147.0 | hsa-miR-200b-3p | −949 |
| hsa-miR-4310 | 136.6 | hsa-miR-27b-3p | −943 |
| hsa-miR-1228-3p | 133.2 | hsa-miR-152-3p | −847 |
| hsa-miR-4655-5p | 118.4 | hsa-miR-10a-5p | −832 |

MicroRNAs highly upregulated in TISE, as calculated by expression in TISE/expression in Islet graft, is shown as Fold upregulation in TISE. MicroRNAs with the lowest expression in TISE compared to Islet graft are shown as Fold down-regulation. Twenty five of the most upregulated and down-regulated microRNAs in TISE are shown.

### Example 5: Detection, Purification and Characterization of Transplant Islet Specific Exosomes in a Type I Diabetic Patient Undergoing Allogeneic Single Donor Islet Cell Transplantation

To understand if the transplant islet specific exosomes platform can be translated to the human clinical setting, plasma EVs were analyzed from a type I diabetic patient (C-peptide negative) undergoing islet transplantation via portal vein infusion of single donor allogeneic islets. As almost every transplant in the clinical setting occurs with a least a single HLA class I mismatch, a single allele mismatch between donor-recipient pair for detection of donor islet specific exosomes in recipient plasma can be utilized (e.g., HLA-A, HLA-B, HLA-A2; Table 3).

TABLE 3
Donor and Recipient HLA Types

| Donor HLA profile | Recipient HLA profile |
|---|---|
| HLA-A2, HLA-A24, HLA-B39, HLA-B61, | HLA-A1, HLA-B8, HLA-C7, HLA-DR17, |
| HLA-C1, HLA-C10, HLA-DR8, HLA-DR16, | HLA-DQB2, HLA-DQA05 |
| HLA-DQB4, HLA-DQB7 | |

### Methods

Recipient underwent a single donor islet transplantation via portal vein infusion. Donor was HLA-A2 and HLA-A24 positive, where as the recipient was HLA-A1 positive at both alleles. Anti-HLA-A2 antibody conjugated beads were utilized to purify donor specific EVs from the recipient patient blood.

For purification of total RNA (including microRNA and mRNA), RNA was extracted from cells and EVs using Trizol, followed by RNeasy mini kit, according to manufacturer's protocol (Qiagen, Germany). For total protein isolation, EV pellet was lysed in 1×RIPA buffer with 1× concentration of protease inhibitor cocktail (Sigma-Aldrich Co., MO).

The xenoislet mouse model was development by the same methods as described in Example 1, and the islet graftectomy was the same method as described in Example 3.

EV enrichment, Western blot, and NanoSight methods were the same as above in Example 2. mRNA expression using reverse transcription-PCR methods were the same as described above for Example 3, but also included human FXYD2γb (forward) 5′-GACAGGTGGTACCTG-3′, (reverse) 5′-CGGCTCATCTTCATTGATTTG-3′(188 bp) primers.

Unconjugated HLA allele specific antibodies (mouse anti-HLA-A2, -HLA-B27, -HLA-B13, -HLA-B8) were purchased from One Lambda (CA, USA), for donor HLA class I donor type specific EV isolation and analysis from recipient plasma.

### Results

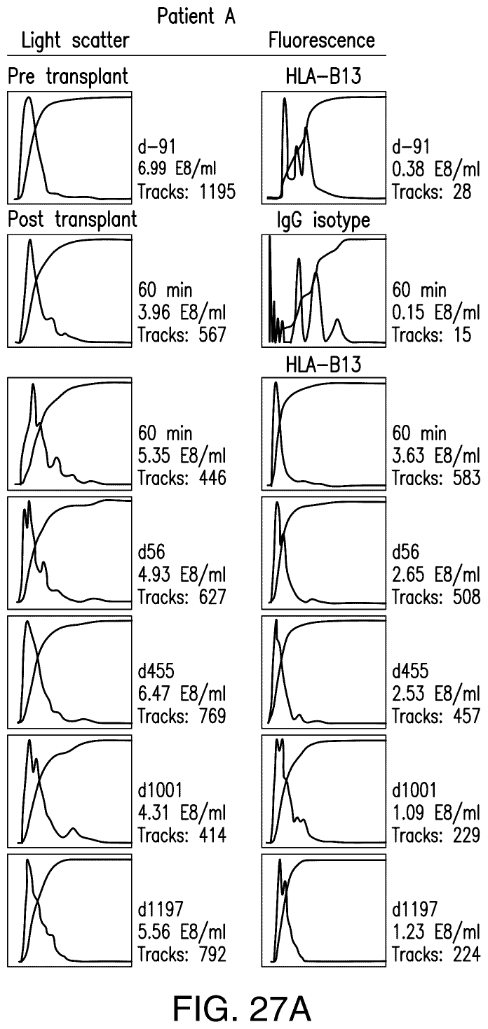

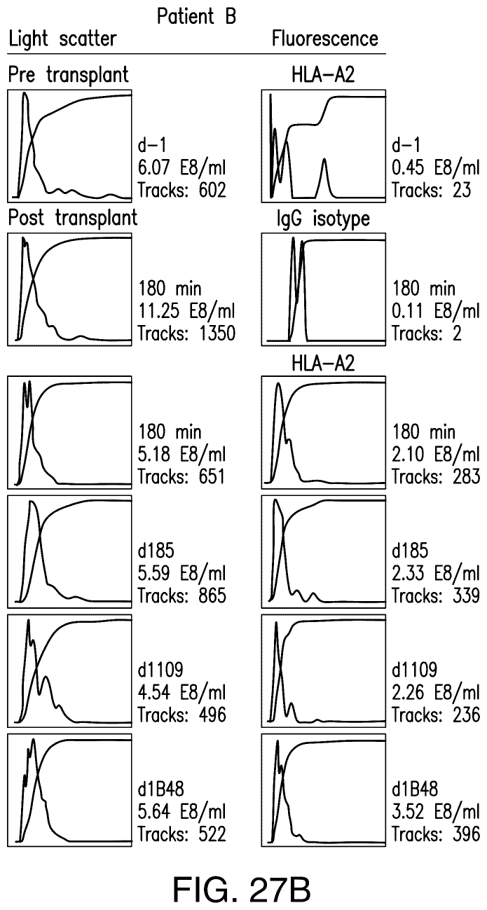

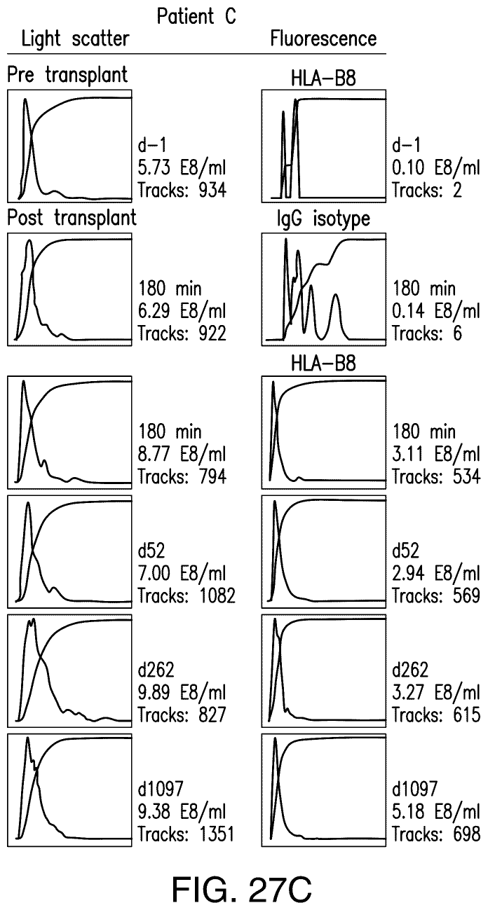

TISE signal was detected in recipient patient plasma taken during long term follow-up visits. In type I diabetic recipient patients (n=4) undergoing single donor infusion of pancreatic islets via the portal vein, pre-transplant, peri-transplant, and long term follow-up (up to 5 years) recipient plasma samples were analyzed for the donor HLA exosome signal using NanoSight fluorescence. Donor-recipient HLA profiles and clinical data (fasting blood glucose, C peptide, type I diabetes autoantibody levels) for Patients A to D are shown in Tables 4-7. In all patients, TISE signal was detected and quantifiable from recipient plasma at every tested post-transplant time point (FIG. 27A-D). In all four patients, pretransplant fluorescence for donor HLA class I was similar to the post-transplant IgG isotype control. Time point of analysis (minutes (min) or days (d #) post-transplant) is shown in each NanoSight panel, with follow-up ranging from 60 minutes to 1848 days.

Graphical representation of the quantified TISE signal in all four patients is shown in FIG. 27E. A decrease in signal is indicative of a decrease in exosome number. In three patients (B-D) the TISE signal plateaued over the follow-up, except for Patient A, where a persistent drop in TISE signal was noted by day 1001 time point.

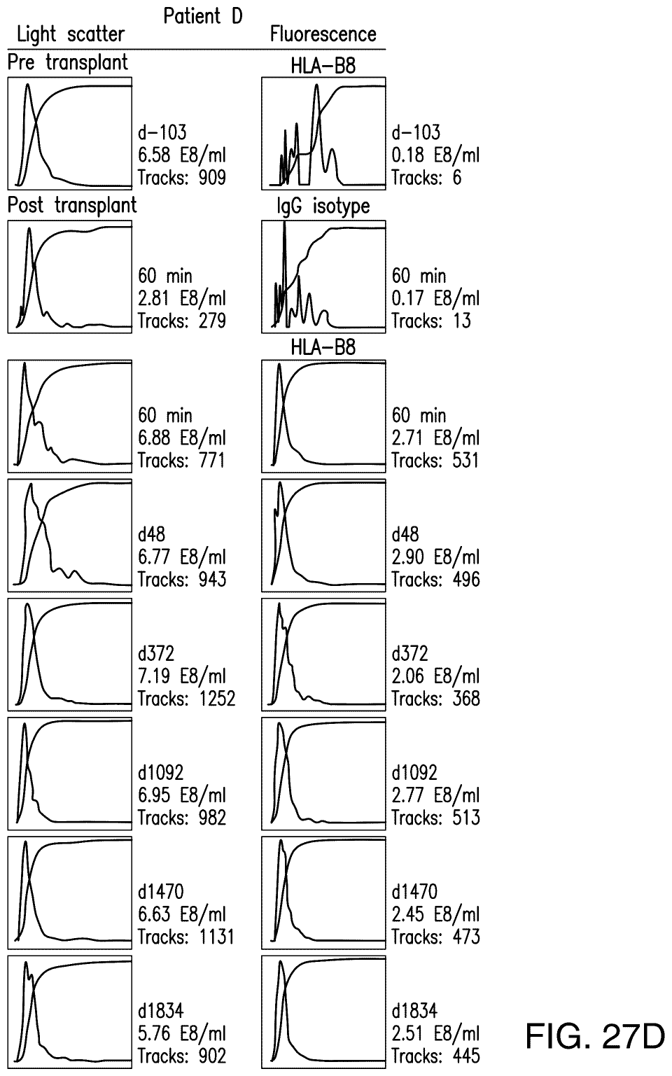

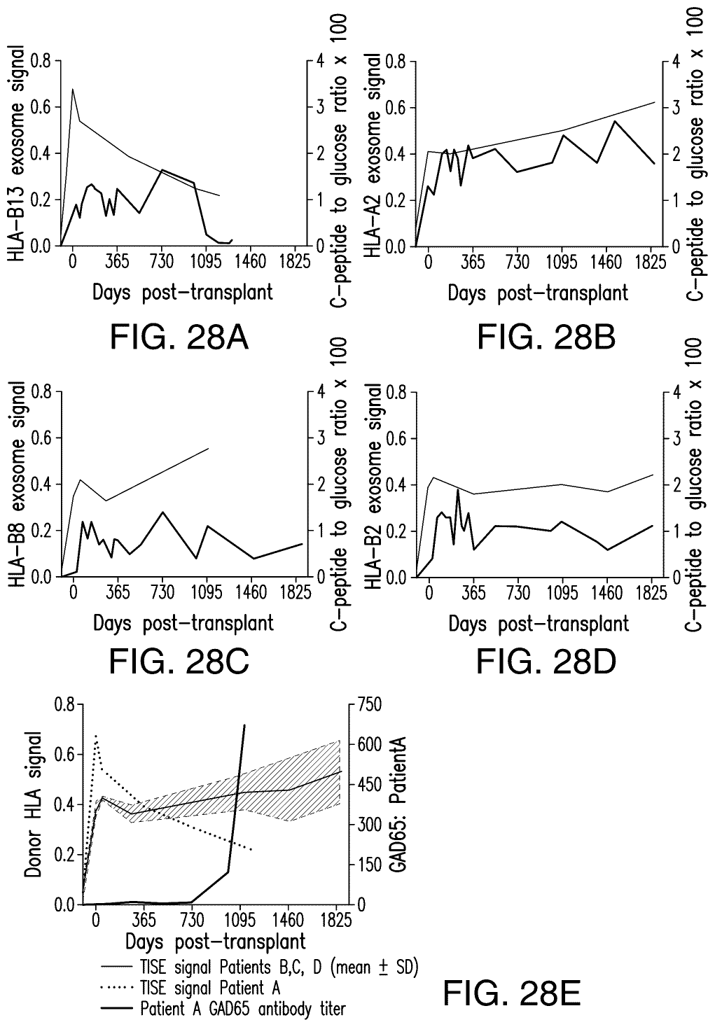

Plasma samples from islet transplant recipients, patients A-D, were analyzed on NanoSight using anti-donor HLA class I specific antibody quantum dot, and the transplant islet exosome signal (FIG. 28A-D; primary y axis, blue (top) line) was quantified over long term follow-up (up to 1848 days post-transplant). In all 4 patients, the pre-transplant sample showed donor HLA exosome signal equivalent to the IgG isotype, but all the post-transplant samples reliably showed long term tracking of the donor specific HLA exosome signal (p=0.0001). Recipient plasma [C-peptide (ng/ml) to glucose (mg/dl) ratio]×100 values over the follow-up period is also shown (FIG. 28A-D; secondary y axis, black (bottom) line). Even though Patient A did not develop an immunologic rejection of the transplanted tissue, this patient developed glutamic acid decarboxylase 65 (GAD65) autoimmune antibody recurrence at this time point without hyperglycemia (FIG. 28E). By day 1197 time point, Patient A had onset of hyperglycemia without evidence of allo-immune rejection (Table 4). The patient developed hyperglycemia due to specific destruction of the transplanted beta cells, not the entire transplanted islet. Thus, the hyperglycemia was caused by destruction of the transplanted beta cells, not from T-cells mediating allo-immune rejection, but from subpopulation of T-cells mediating beta cell autoimmunity. Therefore, in Patient A the decrease in TISE signal temporally correlated with the recurrence of autoimmune antibody, before the clinical manifestation of hyperglycemia.

The contents of the transplanted islet specific exosomes were examined for insulin and glucagon expressing at the protein level. When the GAD65 titer raised by day 1001, the insulin content of the exosomes decreased but the glucagon content did not change.

Taken together, these findings demonstrate that TISE signal can be reliably tracked and quantified over long term follow-up in patients undergoing allogeneic islet transplantation.

TABLE 4
Clinical data, TISE signal, and donor-recipient
HLA profiles for Patient A is shown.

| | | | | C peptide to | | |
|---|---|---|---|---|---|---|
| | | | | glucose ratio | | |
| Interval | | | | [C-peptide | | |
| (days post- | TISE | C-peptide | Glucose | (ng/ml)/glucose | | Insulin |
| transplantat) | signal | (ng/ml) | (mg/dl) | (mg/dl) * 100] | GAD65 | (Units/day) |
| Pre transplant | 0.05 | 0.0 | 162 | 0.03 | 0 | 29 |
| 0 | 0.68 | | | | | |
| 28 | | 0.9 | 122 | 0.91 | | |
| 56 | 0.54 | 0.6 | 96 | 0.59 | | |
| 76 | | 0.9 | 100 | 0.93 | 2 | 0 |
| 119 | | 1.3 | 115 | 1.28 | | 0 |
| 155 | | 1.3 | 101 | 1.33 | 7 | 0 |
| 181 | | 1.2 | 103 | 1.23 | | 0 |
| 238 | | 1.1 | 106 | 1.13 | | 0 |
| 274 | | 0.6 | 97 | 0.64 | 11 | 0 |
| 301 | | 1.0 | 100 | 1.03 | | 0 |
| 336 | | 0.6 | 105 | 0.65 | | 0 |
| 363 | | 1.2 | 100 | 1.24 | 11 | 0 |
| 455 | 0.39 | | | | | 0 |
| 546 | | 0.7 | 100 | 0.71 | 6 | 0 |
| 733 | | 1.6 | 97 | 1.64 | 10 | 0 |
| 992 | | 1.4 | 115 | 1.36 | | 0 |
| 1001 | 0.25 | | | | 125 | 0 |
| 1072 | | 0.6 | 118 | 0.59 | | 0 |
| 1098 | | 0.2 | 105 | 0.24 | 668 | 12 |
| 1197 | 0.22 | 0.1 | 111 | 0.07 | | 17 |
| 1288 | | 0.1 | 160 | 0.06 | | |
| 1302 | | 0.1 | 227 | 0.11 | | 20 |

Histocompatibility Report Patient A

| | A | B | C | Bw | DRB1 | DRw | DQB | DQA | DPB | DPA |
|---|---|---|---|---|---|---|---|---|---|---|
| Patient A | 1 | 8 | 7 | 6 | 4 | 52 | 2 | | | |
| | 2 | 41 | 17 | 6 | 17 | 53 | 2 | | | |
| Donor | 2 | 7 | 6 | 4 | 7 | 53 | 2 | 0102 | | |
| | 3 | 13 | 7 | 6 | 15 | 51 | 6 | 0201 | | |

TABLE 5
Clinical data, TISE signal, and donor-recipient
HLA profiles for Patient B is shown.

| | | | | C peptide to | | |
|---|---|---|---|---|---|---|
| | | | | glucose ratio | | |
| Interval | | | | [C-peptide | | |
| (days post- | TISE | C-peptide | Glucose | (ng/ml)/glucose | | Insulin |
| transplantat) | signal | (ng/ml) | (mg/dl) | (mg/dl) * 100] | GAD65 | (Units/day) |
| Pre transplant | 0.07 | 0.1 | 105 | 0.05 | 112 | 30 |
| 0 | 0.41 | 1.1 | 84 | 1.27 | | |
| 51 | | 0.8 | 76 | 1.05 | | |
| 72 | | 1.3 | 89 | 1.44 | 209 | 0 |
| 115 | | 2.0 | 102 | 1.96 | | 0 |
| 156 | | 1.8 | 85 | 2.08 | | 0 |
| 185 | 0.40 | 1.4 | 88 | 1.57 | 140 | 0 |
| 214 | | 1.8 | 86 | 2.07 | | 0 |
| 242 | | 1.7 | 90 | 1.86 | | 0 |
| 268 | | 1.3 | 94 | 1.34 | 122 | 0 |
| 303 | | 1.6 | 91 | 1.77 | | 0 |
| 331 | | 2.2 | 102 | 2.18 | | 0 |
| 364 | | 1.9 | 100 | 1.85 | 164 | 0 |
| 549 | | 1.9 | 89 | 2.08 | 109 | 0 |
| 730 | | 1.5 | 92 | 1.59 | 110 | 0 |
| 919 | | | | | 100 | 0 |
| 1018 | | 1.7 | 92 | 1.85 | | 0 |
| 1109 | 0.50 | 2.3 | 98 | 2.39 | 115 | 0 |
| 1194 | | | | | | 0 |
| 1384 | | 1.7 | 95 | 1.79 | | 0 |
| 1529 | | 2.6 | 98 | 2.66 | 217 | 0 |
| 1848 | 0.62 | 1.7 | 97 | 1.75 | 63 | 0 |

Histocompatibility Report Patient B

| | A | B | C | Bw | DRB1 | DRw | DQB | DQA | DPB | DPA |
|---|---|---|---|---|---|---|---|---|---|---|
| Patient B | 3 | 18 | 5 | 6 | 4 | 52 | 2 | 03 | | |
| | 68 | 60 | 7 | 6 | 17 | 53 | 8 | 05 | | |
| Donor | 2 | 60 | 10 | 6 | 4 | 52 | 6 | 01 | | |
| | 30 | 60 | 10 | 6 | 13 | 53 | 8 | 03 | | |

TABLE 6
Clinical data, TISE signal, and donor-recipient
HLA profiles for Patient C is shown.

| | | | | C peptide to | | |
|---|---|---|---|---|---|---|
| | | | | glucose ratio | | |
| Interval | | | | [C-peptide | | |
| (days post- | TISE | C-peptide | Glucose | (ng/ml)/glucose | | Insulin |
| transplantat) | signal | (ng/ml) | (mg/dl) | (mg/dl) * 100] | GAD65 | (Units/day) |
| Pre transplant | 0.02 | 0.1 | 169 | 0.03 | 3 | 49 |
| 0 | 0.35 | | | | | |
| 26 | | 0.1 | 62 | 0.08 | | |
| 52 | 0.42 | | | | | |
| 73 | | 1.2 | 98 | 1.24 | 5 | 0 |
| 115 | | 0.8 | 106 | 0.76 | | 0 |
| 143 | | 1.2 | 106 | 1.15 | | 0 |
| 171 | | 1.2 | 120 | 0.96 | 13 | 0 |
| 207 | | 0.8 | 112 | 0.72 | | 0 |
| 241 | | 0.9 | 112 | 0.82 | | 0 |
| 262 | 0.33 | 1.0 | 135 | 0.73 | 13 | 0 |
| 307 | | 0.4 | 93 | 0.39 | | 10 |
| 332 | | 0.9 | 111 | 0.79 | | 5 |
| 360 | | 0.9 | 116 | 0.76 | 20 | 0 |
| 458 | | 0.6 | 111 | 0.50 | | 0 |
| 549 | | 0.7 | 100 | 0.73 | 18 | 0 |
| 731 | | 1.7 | 116 | 1.45 | 7 | 0 |
| 915 | | | | | 9 | 0 |
| 1006 | | 0.4 | 93 | 0.43 | 12 | 0 |
| 1097 | 0.55 | 1.2 | 111 | 1.07 | 10 | 0 |
| 1476 | | 0.5 | 120 | 0.43 | 7 | 0 |
| 1865 | | 0.7 | 107 | 0.69 | 10 | 0 |

Histocompatibility Report Patient C

| | A | B | C | Bw | DRB1 | DRw | DQB | DQA | DPB | DPA |
|---|---|---|---|---|---|---|---|---|---|---|
| Patient C | 1 | 37 | 5 | 4 | 4 | 52 | 3 | | | |
| | 2 | 44 | 6 | 4 | 11 | 53 | 3 | | | |
| Donor | 26 | 8 | 6 | 4 | 11 | 52 | 7 | 01 | | |
| | 30 | 58 | 7 | 6 | 15 | 51 | 6 | 05 | | |

TABLE 7
Clinical data, TISE signal, and donor-recipient
HLA profiles for Patient D is shown.

| | | | | C peptide to | | |
|---|---|---|---|---|---|---|
| | | | | glucose ratio | | |
| Interval | | | | [C-peptide | | |
| (days post- | TISE | C-peptide | Glucose | (ng/ml)/glucose | | Insulin |
| transplantat) | signal | (ng/ml) | (mg/dl) | (mg/dl) * 100] | GAD65 | (Units/day) |
| Pre transplant | 0.03 | 0.1 | 102 | 0.05 | 0 | 27 |
| 0 | 0.39 | | | | 5 | |
| 29 | | 0.5 | 139 | 0.38 | | |
| 40 | 0.43 | 0.6 | 105 | 0.60 | | |
| 75 | | 1.5 | 110 | 1.33 | 6 | 0 |
| 113 | | 1.4 | 105 | 1.36 | | 0 |
| 147 | | 1.3 | 102 | 1.27 | | 0 |
| 180 | | 1.3 | 99 | 1.31 | 11 | 0 |
| 209 | | 0.8 | 115 | 0.70 | | 0 |
| 244 | | 1.9 | 98 | 1.91 | | 0 |
| 274 | | 1.3 | 116 | 1.15 | 17 | 0 |
| 296 | | 1.1 | 104 | 1.02 | | 0 |
| 329 | | 1.2 | 90 | 1.37 | | 0 |
| 372 | 0.36 | 0.7 | 105 | 0.65 | 32 | 0 |
| 547 | | 1.1 | 101 | 1.12 | 25 | 0 |
| 728 | | 1.1 | 103 | 1.06 | 21 | 0 |
| 1004 | | 1.1 | 110 | 1.01 | | 0 |
| 1092 | 0.40 | 1.3 | 115 | 1.17 | 23 | 0 |
| 1367 | | 0.8 | 97 | 0.78 | | 0 |
| 1470 | 0.37 | 0.7 | 112 | 0.60 | 25 | 0 |
| 1834 | 0.44 | 1.6 | 148 | 1.09 | 35 | 0 |

Histocompatibility Report Patient D

| | A | B | C | Bw | DRB1 | DRw | DQB | DQA | DPB | DPA |
|---|---|---|---|---|---|---|---|---|---|---|
| Patient D | 2 | 44 | 3 | 4 | 4 | 52 | 6 | | | |
| | 24 | 62 | 5 | 6 | 13 | 53 | 7 | | | |
| Donor | 3 | 7 | 7 | 6 | 15 | 52 | 2 | 0102 | | |
| | 24 | 8 | 7 | 6 | 17 | 51 | 6 | 0501 | | |

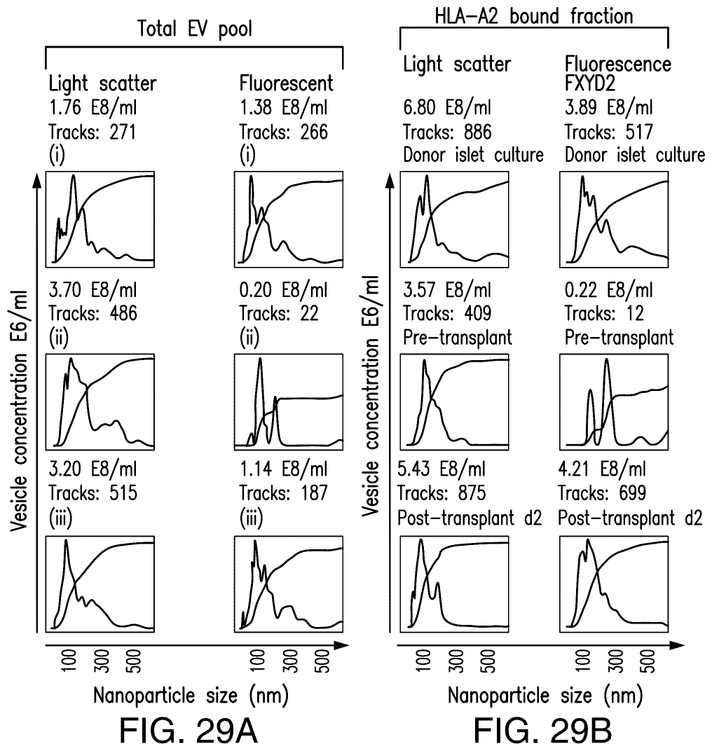

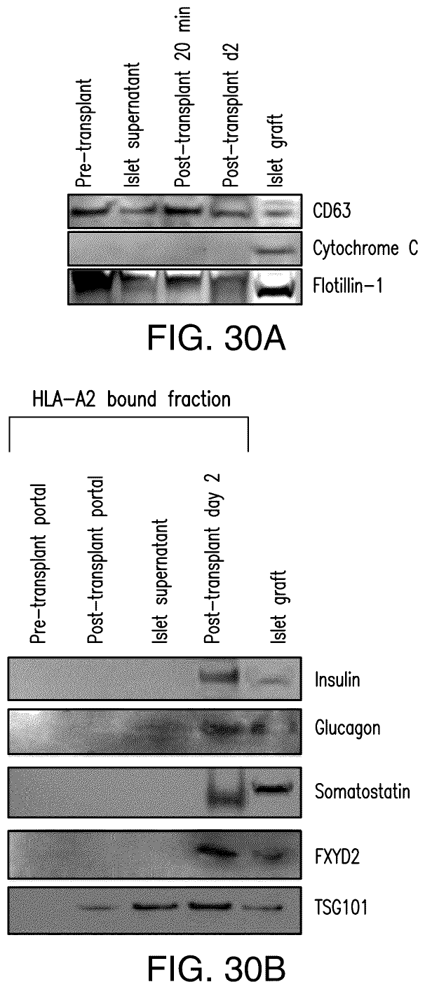

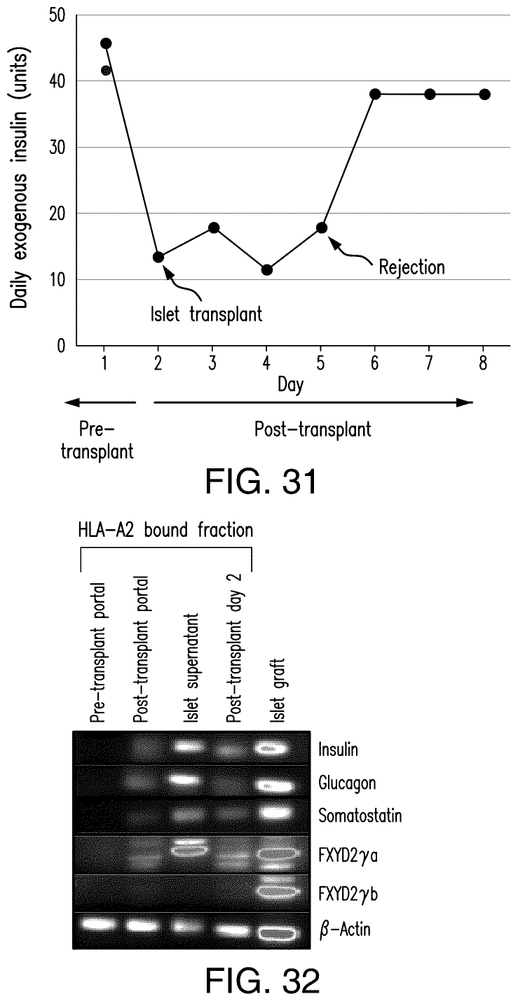

In another patient (Patient E) with type I diabetes undergoing single donor islet transplantation (Donor: HLA-A2+, Recipient: HLA-A2 negative), TISE characterization was performed using anti-HLA-A2 antibody beads. Recipient total plasma EVs were analyzed for donor specific HLA-A2 signal on NanoSight (FIG. 29A). HLA-A2 signal was only seen in recipient post-transplant day 2 plasma sample (iii) and in EVs isolated from donor islet culture supernatant (i), but not in the pre-transplant recipient plasma (ii) sample. HLA-A2 bound EVs were analyzed for FXYD2 expression on NanoSight (FIG. 29B). As confirmation that the isolated microvesicles contained exosomes, samples tested on Western blot showed exosome markers CD63 and flotillin-1, but absence of cellular/apoptotic body marker cytochrome C (FIG. 30A; p=0.008; 1 of 5 shown). Recipient plasma post-transplant day 2 (iii) and donor islet culture supernatant (i) EV fractions showed FXYD2 signal, but the pre-transplant recipient plasma (ii) HLA-A2 bound EV fraction was negative for FXYD2. Western blot analysis showed expression of endocrine hormones and FXYD2 in the recipient post-transplant day 2 peripheral blood plasma HLA-A2 bound EV fractions, but not in the pre-transplant and post-transplant day 0 (20 minutes post-islet infusion) recipient portal vein HLA-A2 bound EV fractions (FIG. 30B), which correlated with the correction of hyperglycemia in the patient (FIG. 31). Islet graft tissue lysate from the xenoislet transplant model served as positive tissue control (labeled as Islet graft). TSG101 is a canonical exosome protein marker.

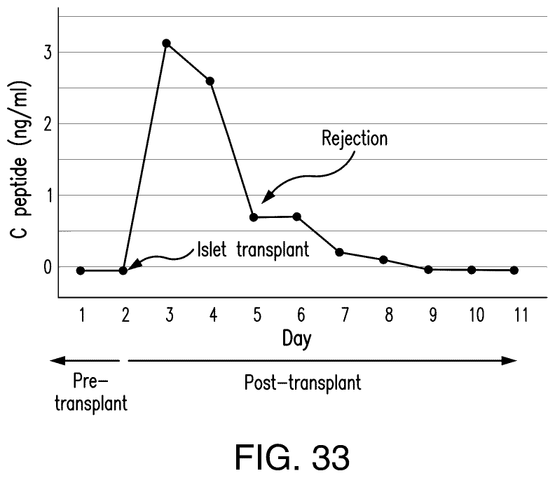

RT-PCR analysis of RNA cargo from HLA-A2 bound EV fractions showed expression of insulin, glucagon, somatostatin, and FXYD2 isoforms (γa and γb) in all the post-transplant recipient plasma samples (day 0 recipient portal vein, post-transplant day 2 peripheral blood), but not in the recipient pre-transplant portal vein sample (FIG. 32). Despite initial C-peptide detection after islet transplantation (representing endogenous insulin production by the transplanted islets), this patient had rapid loss of B-cell function due to acute rejection (FIG. 33). This patient had lower insulin requirements in the immediate post-transplant period, but by day 6 there were signs of acute and complete rejection of the donor islet mass (FIG. 31).

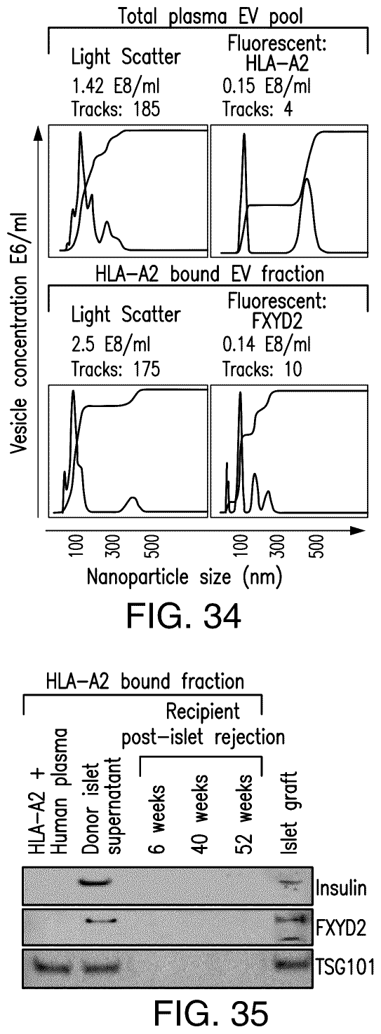

In recipient plasma samples available 6, 40, and 50 weeks after islet-rejection, analysis of total plasma EVs for HLA-A2 expression and the HLA-A2 bound EVs for FXYD2 expression was negative on NanoSight fluorescence (FIG. 34 (representative 6 week sample)). This indicated that the HLA-A2 EV signal was specific to the transplanted donor islets, and complete rejection of the transplanted islets led to loss of the donor specific EV signal in recipient plasma. Likewise, Western blot analysis of the HLA-A2 bound EV fractions from the three different post-islet rejection time points and HLA-A2 positive human plasma failed to show insulin or FXYD2 expression (FIG. 35). Donor islet culture supernatant EVs and xenoislet graft tissue served as positive controls. Third party HLA-A2 positive human plasma EVs from non-diabetic patient were analyzed to make sure that HLA-A2 beads were not non-specifically binding plasma free insulin.

Therefore, in the clinical setting of human islet transplantation, transplant islet specific exosomes can be successfully purified from recipient plasma using anti-donor HLA class I specific allo-antibody, and the transplant islet specific exosomes signal is transplant tissue/cell specific. The TISE signal is specific to the cellular constituents of the transplanted islets. Similar to the xenoislet model, TISE co-express islet surface markers and carry specific endocrine hormone proteins and mRNAs as part of their intra-exosomal cargo in the human transplant setting.

### Example 6: Detection of GAD65 and ZnT8 Protein in Islet Beta Cell Exosomes in a Xenoislet Transplant Model as a Marker for Type I Diabetes

Type I diabetes is an autoimmune disorder with progressive islet beta cell destruction, associated with development of several autoantibodies, including those against insulin, GAD65, and ZnT8. In particular, GAD65 is one of the major islet autoantigens that is implicated in type I diabetes, and currently anti-GAD65 antibodies are assessed as one of the potential prognosticators for type I diabetes development. This study examined whether the autoantigens GAD65 and ZnT8 are expressed in purified beta cell exosomes isolated from peripheral blood. These are specific for beta cells, and more and more specifically in type I diabetes they are implicated in development of the disease as patients develop autoantibodies to these beta cell antigens.

In the xenoislet transplant mouse model (human islets transplanted into athymic, diabetic mouse), putative islet specific exosomes were isolated from recipient mouse plasma exosomes using anti-HLA-A beads, as outline in Example 2. A graftectomy was also performed, as outlined in Example 3.

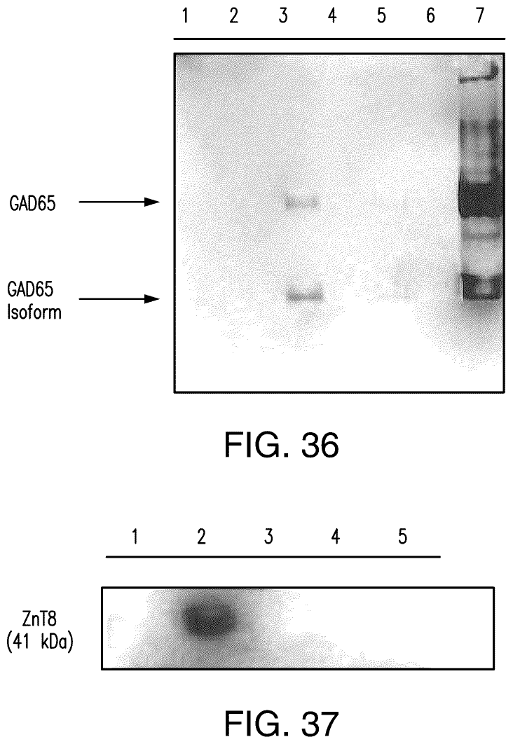

In the xenoislet transplant mouse model, GAD65 staining on exosomes was noted only in the islet beta cell enriched exosomes, but not other conditions (FIG. 36). GAD65 was also scen in the islet graft cell lysate, as expected. Likewise, ZnT8 staining on exosomes was noted only in the beta cell exosomes, but not other conditions (FIG. 37).

This data, together with Example 3 (demonstrating the expression of insulin and FXYD2 in purified exosomes isolated from the peripheral blood of the xenoislet transplant mouse model) demonstrates that beta cell exosomes can indeed be characterized from peripheral blood and can therefore be used as the basis of a biomarker platform, e.g., platform for use in the context of monitoring diabetes.

### Example 7: Quantitative Changes in Transplant Tissue Specific Exosome Number as a Biomarker to Monitor Transplant Rejection

The xenoislet transplantation mouse model was used to understand changes in transplant tissue specific exosome profiles from recipient plasma under condition of rejection.

In five athymic nude, diabetic mice that underwent xenoislet transplantation, strain matched leukocytes (NU/J strain wild type 2×10⁷ cells/animal) were injected into their peritoneal cavity to cause islet rejection. All five animals rejected the human islet grafts as confirmed by histology. The first day that the recipient glucose increased to >200 mg/dL (checked twice over a 4 hour interval), the animal was sacrificed and its plasma exosome pool was analyzed for donor islet signal. These were compared to the control animals.

Exosomes were isolated from recipient plasma, and the plasma exosomes were analyzed for transplant human islet signal using anti-HLA-A antibody quantum dot on NanoSight nanoparticle detector. IgG control represents isotype antibody signal in a xenoislet animal, and represents background fluorescence signal. Representative fluorescence for HLA-A positive exosomes is shown in two xenoislet animals without rejection and five xenoislet transplant mouse model in which islet graft rejection was induced by intraperitoneal infusion of recipient matched leukocytes into the animals.

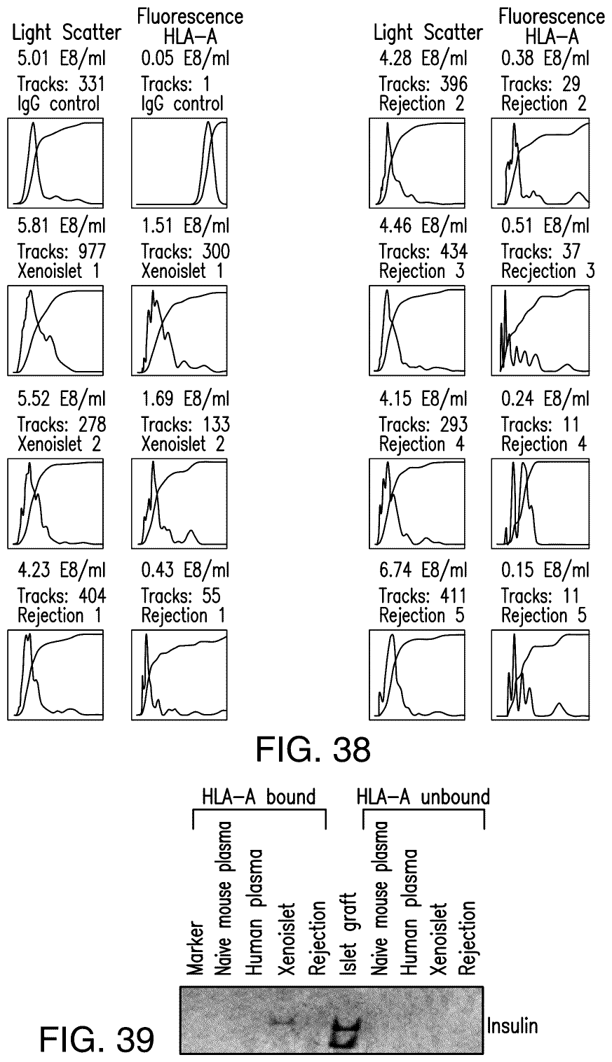

NanoSight fluorescence values in the two control animals and five rejection animals are shown in FIG. 38. In all five rejection animals, the rejection animals and control animals expressed the same number of total exosomes in the plasma. The donor human islet specific signal positive exosomes, however, were dramatically reduced in the five rejection animals as compared to the control animals. The HLA-A signal in all five rejection animals was at least 2.5 times lower than the xenoislet animals (Xenoislet mean HLA-A signal 0.285+0.048; Rejection mean HLA-A signal 0.067+0.037; p<0.001) demonstrating that quantitative change in the transplant tissue specific exosome signal can serve as a biomarker to monitor transplant rejection.

Rejection of the xenoislet graft leads to loss of insulin positive exosomes. Compared to the control animals (islet specific hormone protein), which clearly showed insulin expression on Western blot analysis, the rejection animal sample showed a dramatic decrease in insulin expression (FIG. 39). As expected, insulin was not detected in the bound and unbound negative controls (naive mouse plasma and human plasma). HLA-A bound exosomes showed expression of insulin in xenoislet sample, but not in the HLA-A unbound exosomes, showing that the transplant islet signal was specific to the HLA-A positive exosomes (donor specific). These results validate the finding that transplant rejection causes a dramatic decrease in transplant tissue specific exosome expression and therefore decrease in transplant tissue specific markers (insulin in this case), even though the total plasma exosome numbers remain the same.

### Example 8: Quantitative Changes in Transplant Tissue Specific Exosome Number and Content as a Biomarker to Monitor Transplant Rejection

The xenoislet transplantation mouse model was further studied to measure changes in transplant tissue specific exosome profiles from recipient plasma under condition of rejection.

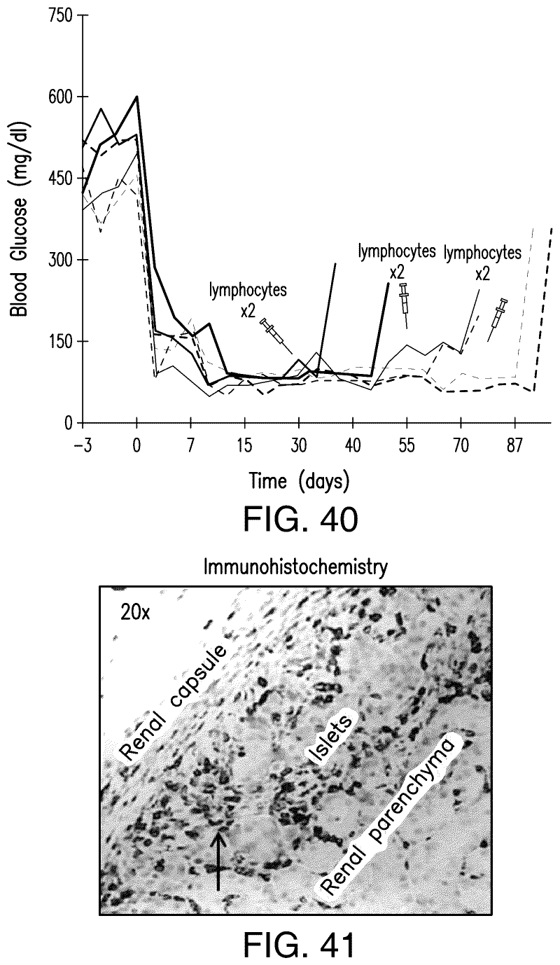

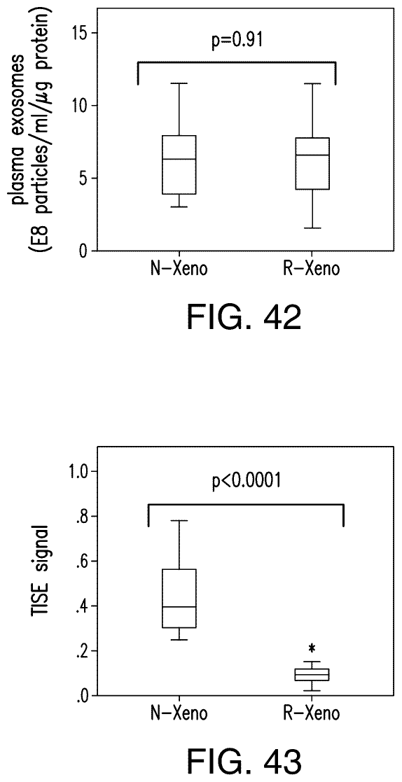

Immune rejection was induced in the normoglycemic xenoislet recipients (R-xeno, n=15) by intra-peritoneal infusion of recipient mouse MHC matched (NU/J strain) syngeneic leukocytes (NU/J strain wild type 2×10⁷ cells/animal) (FIG. 40). Animals were sacrificed on the first day that the plasma glucose increased to >200 mg/dL (checked twice over a 3 hour interval). Histology confirmed infiltration of the islet graft by CD3 positive T-cells, with decrease in the islet mass (FIG. 41). As control, 15 xenoislet recipients received intra-peritoneal infusion of saline and sacrificed 15 days later (N-xeno). On NanoSight, there were no quantitative differences in the total plasma exosome numbers between the R-xeno versus N-xeno (FIG. 42). The HLA-A signal in the R-xeno plasma exosomes was assessed and a signal reduction was noted in all samples. Quantitation of HLA-A signal demonstrated that immune rejection leads to significant loss in the plasma TISE signal (p<0.0001; FIG. 43). Collectively, these data demonstrated that transplanted human islets release a distinct donor MHC specific exosome signal into recipient plasma that can be serially tracked and quantified over the course of post-transplant follow-up. TISE signal is transplant tissue specific and immune rejection leads to a significant quantifiable drop in TISE numbers that correlates with the clinical picture of first-onset hyperglycemia and histological evidence of islet rejection.

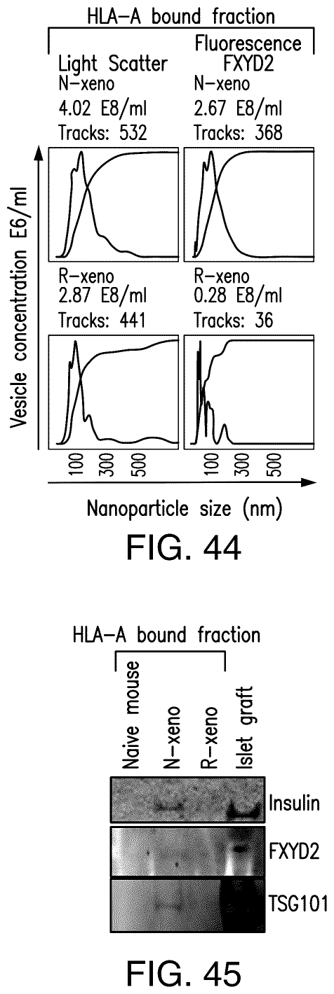

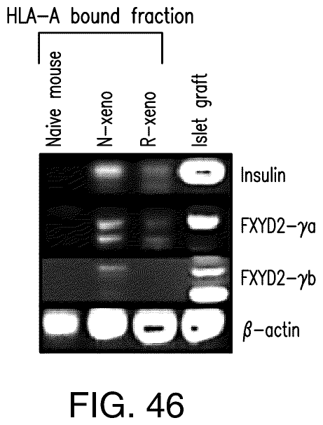

TISE expression of islet endocrine hormones was assessed under conditions of immune rejection (R-xeno). On NanoSight fluorescence, R-xeno sample showed decreased co-expression of FXYD2 (FIG. 44). On Western blot, a faint signal for insulin and FXYD2 was detected (FIG. 45). RT-PCR showed decrease in insulin mRNA signal in R-xeno compared to N-xeno sample for the same amount of total RNA per sample (FIG. 46). Collectively, these data indicate that immune rejection leads to decreased detection of TISE markers in the HLA-A bound exosome fraction.

### Example 9: Detection of Changes in TISE Proteomic and RNA Cargoes Following Immune Rejection in an Islet Cell Transplantation Model

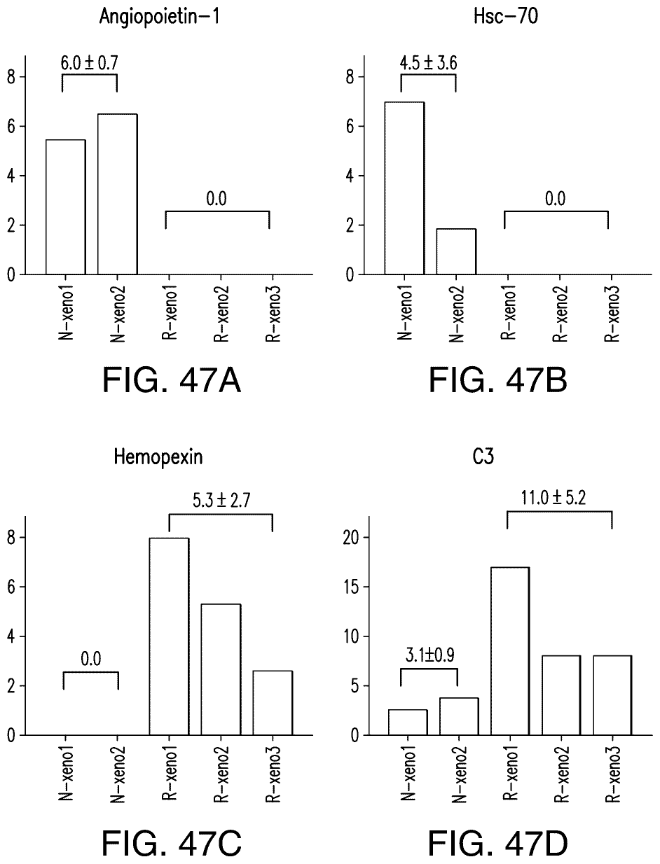

Having demonstrated a quantitative drop in TISE numbers with transplant rejection, it was assessed whether this leads to changes in its proteomic and RNA profiles (TISE N-xeno (animals without immunologic rejection) versus R-xeno (animals undergoing rejection)). Proteomic profiles of TISE from three independent R-xeno experiments were compared to the two N-xeno samples to look for consistent differences between rejection versus normal conditions. Proteins were selected that were either absent or expressed at very low levels in the two N-xeno samples compared to the three R-xeno samples, and vice versa. It was confirmed that the identified proteins were human derived, not mouse derived, by comparing all peptide matches for a given protein on the NCBI protein blast against human and mouse forms of the protein of interest. Four human proteins showed consistent expression differences between normal versus rejection conditions—heat shock cognate protein 71 (Hsc-70), angiopoietin-1, hemopexin, and complement C3 (FIG. 47 A-D).

Small RNA profiling of R-xeno samples was performed. Because of the low TISE levels from a single sample TISE from five animals had to be pooled for microarray analysis. Many microRNAs expressed in R-xeno sample were also highly expressed in N-xeno sample. microRNAs for each array were compared that showed at least 2 fold or greater expression over the median value for that microarray set. The list of microRNAs differentially upregulated in R-xeno TISE compared to N-xeno TISE is shown in Table 8. Small RNA profiling data from TISE enriched from R-xeno sample was compared to TISE from N-xeno sample. MicroRNAs with >2 fold expression over median value from R-xeno sample were compared to their relative expression in N-xeno sample. Expression in each sample was normalized to the internal median value as shown, along with the relative expression for that microRNA in R-xeno (TISE R-xeno/TISE N-xeno).

Of the four human TISE proteins noted to be different in conditions of no rejection versus rejection, angiopoietin-1 and heat shock protein 71 kDa, which were only seen at high levels in TISE from N-xeno animals, have both been reported to play important protective roles in islet physiology. Angiopoietin-1 production in islets was noted to improve revascularization after transplantation and to protect islets from cytokine induced apoptosis. In the 3 R-xeno samples, a complete absence of angiopoietin-1 and heat shock protein 71 kDa was noted, but elevated levels of complement C3 and hemopexin were found. Expression of complement C3 protein on donor tissue has been shown to be necessary for T-cell mediated transplant organ rejection, and elevated hemopexin levels have been seen in donor graft tissue under conditions of rejection. Further, TISE microarray analysis showed upregulation of several microRNAs in the R-xeno sample compared to the N-xeno sample; but interestingly the majority of microRNAs were common under both conditions.

Collectively, these data demonstrate that immune rejection alters the proteomic and RNA cargoes of TISE.

TABLE 8
Immune rejection of transplanted islets changes
the microRNA profiles in TISE

| | | Expression R- | Expression | Fold |
|---|---|---|---|---|
| miRNA | Type | Xeno | N-Xeno | enrichment |
| hsa-mir-4722 | stem-loop | 7.5 | 1.1 | 6.7 |
| hsa-miR-4722-3p | miRNA | 9.5 | 1.8 | 5.3 |
| hsa-miR-6764-5p | miRNA | 4.1 | 0.9 | 4.6 |
| hsa-mir-516a-1 | stem-loop | 1.8 | 0.6 | 3.1 |
| hsa-mir-516a-2 | stem-loop | 1.8 | 0.6 | 3.1 |
| hsa-miR-5093 | miRNA | 2.1 | 0.7 | 3.0 |
| hsa-miR-4786-3p | miRNA | 3.2 | 1.1 | 2.9 |
| hsa-miR-4281 | miRNA | 7.5 | 2.6 | 2.8 |
| hsa-mir-8087 | stem-loop | 1.4 | 0.5 | 2.6 |
| hsa-miR-4656 | miRNA | 2.1 | 0.8 | 2.6 |
| hsa-mir-1225 | stem-loop | 1.6 | 0.7 | 2.5 |
| hsa-mir-5681b | stem-loop | 1.9 | 0.8 | 2.4 |
| hsa-mir-135b | stem-loop | 1.4 | 0.6 | 2.3 |
| hsa-mir-5683 | stem-loop | 1.6 | 0.7 | 2.3 |
| hsa-mir-4755 | stem-loop | 1.4 | 0.6 | 2.3 |
| hsa-miR-4729 | miRNA | 1.4 | 0.6 | 2.3 |
| hsa-miR-4481 | miRNA | 1.4 | 0.6 | 2.2 |
| hsa-miR-1229-5p | miRNA | 1.6 | 0.7 | 2.2 |
| hsa-miR-3184-3p | miRNA | 2.1 | 1.0 | 2.2 |
| hsa-miR-1307-3p | miRNA | 1.6 | 0.8 | 2.2 |
| hsa-miR-573 | miRNA | 1.6 | 0.8 | 2.2 |
| hsa-mir-570 | stem-loop | 1.6 | 0.8 | 2.1 |
| hsa-miR-4720-3p | miRNA | 1.6 | 0.8 | 2.1 |
| hsa-miR-4642 | miRNA | 1.4 | 0.7 | 2.1 |
| hsa-miR-3115 | miRNA | 1.4 | 0.7 | 2.1 |
| hsa-mir-6759 | stem-loop | 1.6 | 0.8 | 2.1 |
| hsa-miR-5001-5p | miRNA | 1.6 | 0.8 | 2.1 |
| hsa-mir-4665 | stem-loop | 1.6 | 0.8 | 2.0 |
| hsa-miR-3187-5p | miRNA | 1.9 | 0.9 | 2.0 |
| hsa-mir-3137 | stem-loop | 1.6 | 0.8 | 2.0 |
| hsa-miR-4776-3p | miRNA | 1.6 | 0.8 | 2.0 |
| hsa-miR-6768-5p | miRNA | 1.4 | 0.7 | 2.0 |
| hsa-mir-4739 | stem-loop | 1.4 | 0.7 | 2.0 |
| hsa-miR-513c-3p | miRNA | 1.4 | 0.7 | 2.0 |
| hsa-miR-7855-5p | miRNA | 1.9 | 0.9 | 2.0 |
| hsa-mir-6835 | stem-loop | 1.6 | 0.8 | 2.0 |
| hsa-miR-664a-3p | miRNA | 1.4 | 0.7 | 2.0 |
| hsa-miR-4771 | miRNA | 1.4 | 0.7 | 2.0 |
| hsa-mir-3689a | stem-loop | 1.6 | 0.8 | 2.0 |

### Example 10: Kidney Human Transplant Model

Donor kidney specific exosomes were assessed from plasma and urine samples in patients undergoing living donor renal transplantation (n=3). Representative results from a single donor-recipient pair are shown (donor: HLA-A2, HLA-B27; recipient: HLA-A29, HLA-A31, HLA-B44).

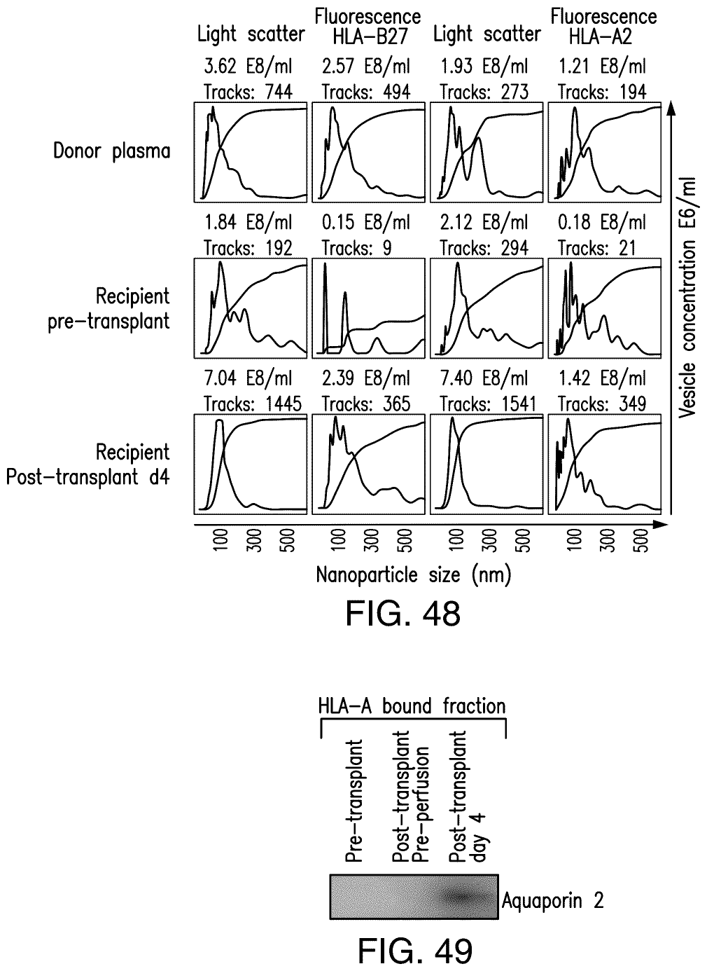

NanoSight fluorescence for HLA-A2 and HLA-B27 showed strong signals in the Donor plasma (positive control) and Recipient post-transplant d4 (day 4) plasma samples, but not in the Recipient pre-transplant sample. (FIG. 48). Western blot analysis of post-transplant HLA-A2 day 4 sample bound plasma exosome fraction confirmed expression of renal epithelial protein, aquaporin 2 (FIG. 49), but not in the pre-transplant-thus attesting to the validity of transplant tissue specific exosome characterization for other tissue types (e.g. kidney). Intraoperative recipient plasma obtained after kidney implantation, but before organ perfusion (labeled post-transplant pre-perfusion) was also negative for aquaporin 2 expression.

These findings were also validated in recipient urine exosomes. Urinary exosome isolation was performed as described elsewhere with slight modification (Pisitkun T. et al., Proc Natl Acad Sci USA, 2004 101:13368; Rood I. M., et al., Kidney Int, 2010 78:810). Briefly, urinary cell debris was removed from 20 ml starting material by centrifugation at 17,000 g for 15 minutes. The supernatant was then ultracentrifuged at 200,000 g for 120 minutes at 4° C. The pellet was resuspended in PBS and loaded onto a Sepharose 2B size exclusion column, and the eluted fractions representing exosomes were pooled. The pooled fractions were concentrated on an Amicon filter (Merck Millipore Ltd., Ireland) with 100 kDa cut-off membrane.

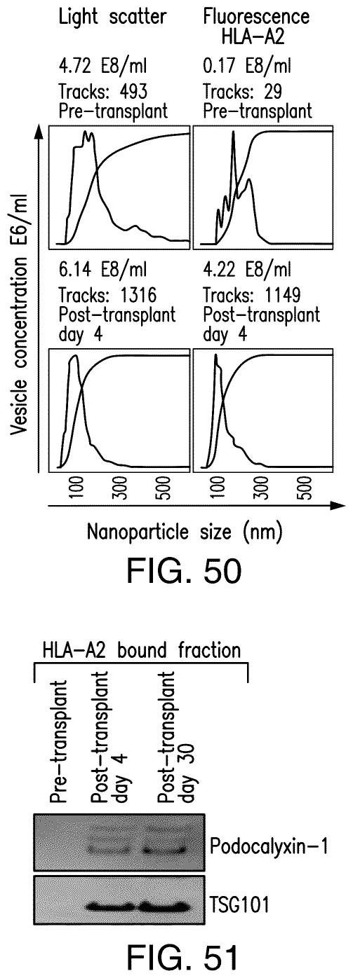

Urine samples (40 ml) were collected in sterile cups, treated with 1× protease inhibitor cocktail (Sigma Aldrich) and frozen at −80° C. Post-transplant urine sample showed donor specific HLA-A2 positive exosomes on NanoSight fluorescence (FIG. 50), and anti-HLA-A2 antibody bead bound urinary exosome fraction from post-transplant days 4 and 30 showed presence of renal glomerular protein, podocalyxin-1 (FIG. 51). Enrichment of renal proteins was not seen in the pre-transplant urine samples using anti-HLA-A2 antibody beads.

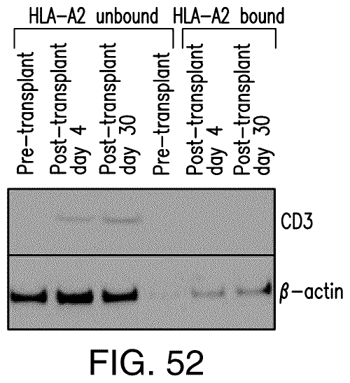

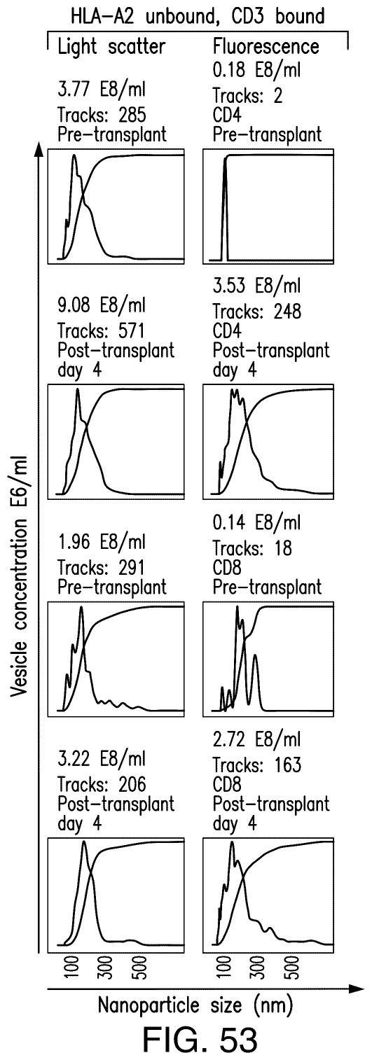

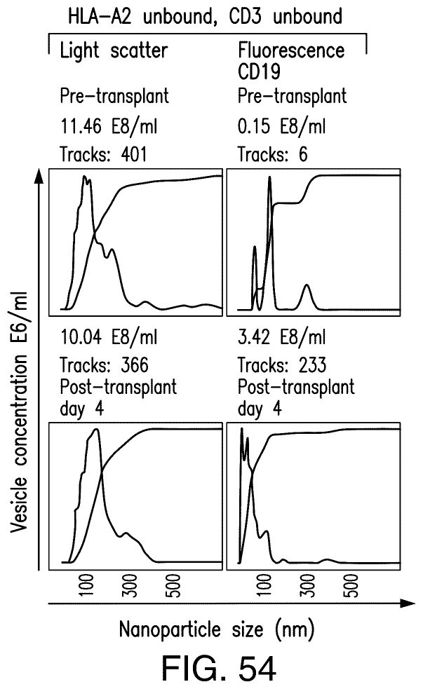

The feasibility of characterizing T-cell specific exosome fraction from recipient urine was assessed, which may further improve the diagnostic accuracy and time sensitivity of the biomarker platform. It was noted that CD3 positive exosome signal representing the T-cell specific exosome fraction could be detected in recipient urine HLA-A2 unbound exosomes in the post-transplant samples but not the pre-transplant sample (FIG. 52). Using anti-CD3 antibody conjugated beads a T-cell exosome subset was purified. This subset was positive for surface co-expression of helper T-cell (CD4) and cytotoxic T-cell (CD8) markers on NanoSight fluorescence (FIG. 53). Further, post-transplant HLA-A2 unbound, CD3 unbound urine exosomes were positive for B-cell surface marker, CD19, on NanoSight fluorescence (FIG. 54).

Taken together these findings support the concept that in addition to transplant tissue specific exosome characterization, immune cell specific exosome quantitation and characterization can be successfully performed from patient bodily fluids.

### Example 11: FXYD2 Enriched Exosome Platform as a Diagnostic Assay for Monitoring Islet Beta Cell Status

This example demonstrates that islet exosomes show differences in exosome profiles in type I diabetic patients as compared to normal controls. FXYD2 has two isoforms, γA and γB, which are expressed on the surface of islet beta cell exosomes. FXYD2 γA antibody was used as a marker to enrich islet beta cell exosomes from patient blood. It was found that insulin containing exosomes were enriched as compared to non-FXYD2 exosomes.

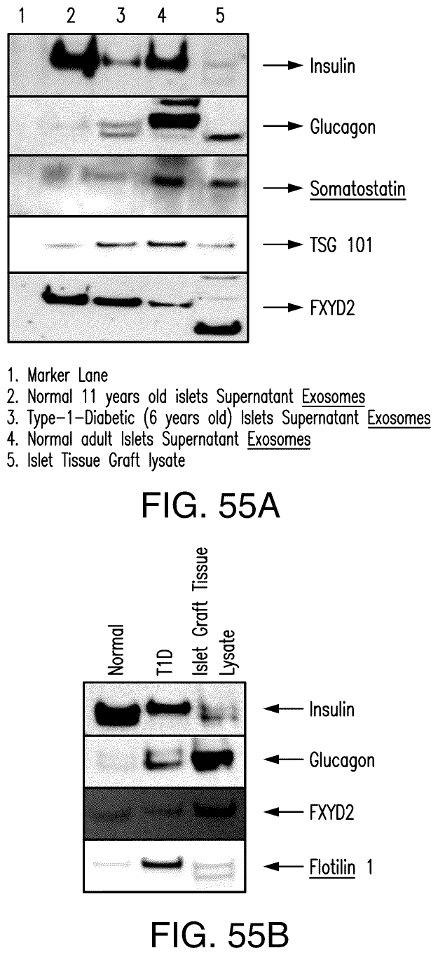

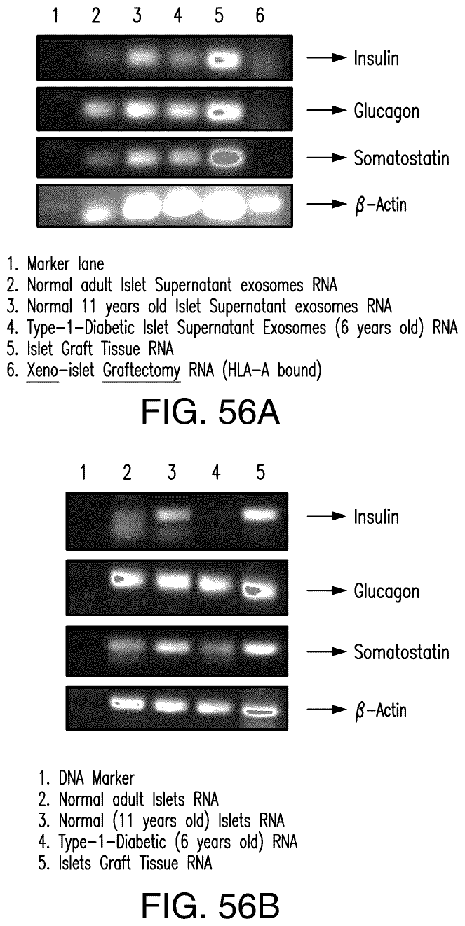

To validate this concept exosomes isolated from supernatant media of islet cultures in vitro were first studied. First, it was found that exosomes from normal subject islet culture show higher levels of insulin on Western blot (FIG. 55A) and RT-PCR (FIG. 56A) as compared to an age matched type 1 diabetic patient islet exosomes obtained from in vitro culture. Islet graft tissue was used as positive control. For FIG. 55A, type 1 diabetic islets (6 year old) showed decreased insulin expression compared to age matched control (11 year old) and normal adult control. Insulin levels from adult islet exosomes are also shown. TSG101 is a canonical exosome marker protein. FXYD2 expression is also shown. For FIG. 56A, type 1 diabetic islets (6 year old) showed decreased insulin mRNA expressed in exosomes from cultured supernatants of in vitro islet cultures as compared to age matched control (11 year old) and normal adult control. But expression of glucagon and somatostatin mRNAs was similar in all three subjects. FIG. 56B uses RT-PCR to examine islet endocrine hormone RNA in vitro cultured islets themselves (i.e., not the exosomes). Type 1 diabetic islets (6 years old) showed decrease insulin mRNA compared to age matched control (11 year old) and normal adult control. But expression of glucagon and somatostatin mRNA was similar in all three subjects.

In FIG. 55B, another type 1 diabetic (TID) patient (38 years old) with endogenous insulin production and an age-matched control (36 years old), islet culture supernatant exosomes were analyzed on Western blot for insulin, glucagon, and FXYD2 content. Similar to the other type 1 diabetic patient, this patient also demonstrated decreased insulin and FXYD2 content as compared to age matched control, while glucagon levels remained the same or higher in the type 1 diabetic patient.

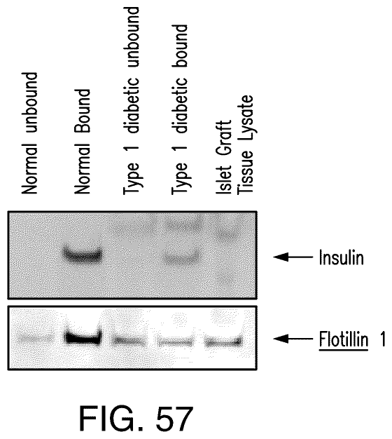

Next, it was studied whether insulin containing exosomes (representing exosomes released by beta cells) could be enriched using FXYD2 γA antibody beads. Compared to the bead unbound exosomes, the bead bound exosomes showed expression of insulin on Western blot (FIG. 57). Exosomes from the in vitro islet culture supernatants were incubated with FXYD2 γA antibody conjugated beads, and the bound and unbound fractions were analyzed on Western blot for insulin protein enrichment. In exosomes from both type 1 diabetic donor and normal control subject, insulin containing exosomes were enriched in the bound fraction compared to the unbound fraction. Increased insulin was seen in the normal subject bound fraction. Flotillin is a canonical exosome marker protein. Islet graft tissue was used as a positive control.

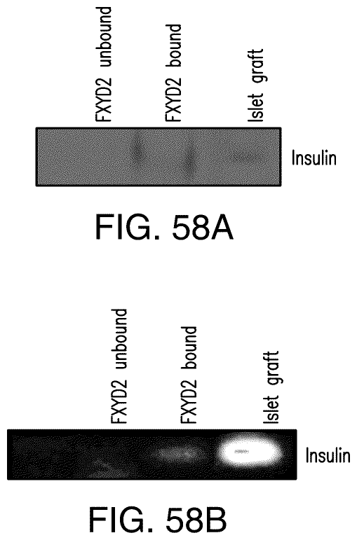

Given these findings in the in vitro setting, it was analyzed whether FXYD2 γA antibody bound beads could enrich insulin containing exosomes from human plasma. First, it was studied whether FXYD2 γA bead bound exosomes show expression of FXYD2 on their surface, as compared to the unbound exosomes, to validate enrichment of FXYD2 expressing exosomes. In another pancrease donor, islet exosomes were prepared from culture supernatant and then incubated with FXYD2 γA antibody beads. The bead bound and unbound fractions were analyzed on Western blot (FIG. 58A) and RT-PCR (FIG. 58B) for enrichment of insulin containing exosomes.

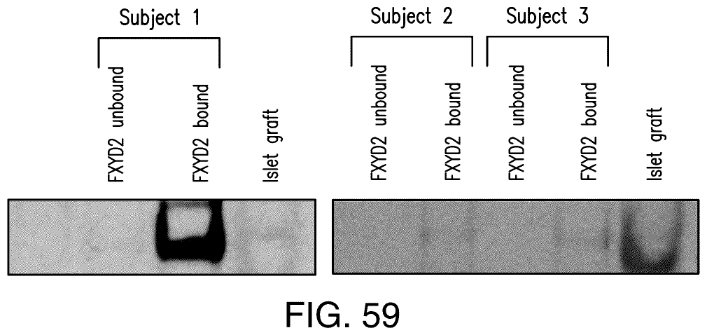

Given this validation, it was tested whether FXYD2 γA bead bound exosomes were enriched for insulin expression as part of its intra-exosomal cargo. In three normal subjects, the bead bound exosomes showed enrichment of insulin protein on Western blot (FIG. 59). Here plasma exosomes were isolated and incubated with FXYD2 YA antibody beads. The bead bound and unbound exosome fractions were analyzed on Western blot for insulin enrichment. In all three cases, the FXYD2 γA bead bound exosomes showed enrichment of exosomes, demonstrating the enrichment of islet beta cell exosomes from human plasma. Islet graft tissue served as positive control.

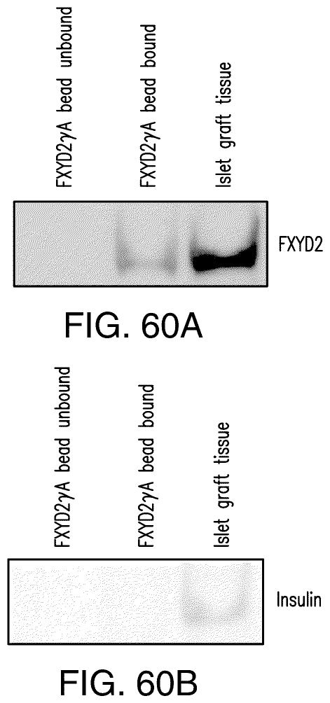

A similar analysis was conducted in a subject with type 1 diabetes, where it was found that the FXYD2 bound exosomes did not enrich for insulin expression, similar to the unbound exosomes. FIG. 60 presents a type 1 diabetic adult patient in early stage of the disease, it was assessed whether FXYD2γA antibody bead bound exosomes showed decreased expression of insulin as compared to normal subjects. Islet graft tissue served as positive control. FIG. 60A shows that FXYD2γA antibody beads do enrich for FXYD2γA protein as part of its protein cargo as demonstrated by the fact that as compared to the unbound fraction, the FXYD2γA bound fraction eluted exosomes showed high expression of FXYD2γA. In FIG. 60B, FXYD2γA bound and unbound exosomes were analyzed for insulin expression. Neither the bound nor the unbound exosome fractions enriched for insulin expression. This shows that type 1 diabetic patients express lower levels of beta cell exosomes containing insulin even at the early stages of the disease.

It was also noted that FXYD2 γA positive exosome profiles change with glucose stimulation, as evidenced by proteomic profiles that were recently performed looking at the proteomic cargo in FXYD2 γA bead bound exosomes under conditions of fasting versus stimulation with a carbohydrate rich diet (Table 9). Changes were seen in the proteomic cargo with carbohydrate stimulation.

TABLE 9
Proteomic profiles of fasting versus carbohydrate rich diet

| | | | 60 |
|---|---|---|---|
| | Identified Proteins (259) | Fasting | minutes |
| 1 | Apolipoprotein C-III OS = Homo sapiens GN = APOC3 PE = 1 | 159 | 109 |
| | SV = 1 | | |
| 2 | Ig gamma-1 chain C region OS = Homo sapiens GN = IGHG1 | 84 | 84 |
| | PE = 1 SV = 1 | | |
| 3 | Apolipoprotein B-100 OS = Homo sapiens GN = APOB PE = 1 | 97 | 81 |
| | SV = 2 | | |
| 4 | Isoform 2 of Filamin-A OS = Homo sapiens GN = FLNA | 75 | 76 |
| 7 | von Willebrand factor OS = Homo sapiens GN = VWF PE = 1 | 77 | 52 |
| | SV = 4 | | |
| 5 | Talin-1 OS = Homo sapiens GN = TLN1 PE = 1 SV = 3 | 79 | 68 |
| 16 | Ig mu chain C region OS = Homo sapiens GN = IGHM PE = 1 | 42 | 36 |
| | SV = 3 | | |
| 10 | Hemoglobin subunit beta OS = Homo sapiens GN = HBB PE = 1 | 52 | 48 |
| | SV = 2 | | |
| 9 | Ig kappa chain C region OS = Homo sapiens GN = IGKC PE = 1 | 47 | 49 |
| | SV = 1 | | |
| 8 | Isoform 2 of Fibrinogen alpha chain OS = Homo sapiens | 21 | 50 |
| | GN = FGA | | |
| 12 | Actin, cytoplasmic 1 OS = Homo sapiens GN = ACTB PE = 1 | 43 | 39 |
| | SV = 1 | | |
| 6 | Alpha-2-macroglobulin OS = Homo sapiens GN = A2M PE = 1 | 28 | 54 |
| | SV = 3 | | |
| 14 | Myosin-9 OS = Homo sapiens GN = MYH9 PE = 1 SV = 4 | 41 | 38 |
| 20 | Hemoglobin subunit alpha OS = Homo sapiens GN = HBA1 | 35 | 32 |
| | PE = 1 SV = 2 | | |
| 15 | Ugl-Y3 OS = Homo sapiens GN = FN1 PE = 2 SV = 1 | 31 | 38 |
| 17 | Protein APOC4-APOC2 OS = Homo sapiens GN = APOC4- | 46 | 36 |
| | APOC2 PE = 4 SV = 1 | | |
| 23 | Ig gamma-3 chain C region OS = Homo sapiens GN = IGHG3 | 30 | 28 |
| | PE = 1 SV = 2 | | |
| 18 | Apolipoprotein C-I OS = Homo sapiens GN = APOC1 PE = 1 | 21 | 34 |
| | SV = 1 | | |
| 19 | Apolipoprotein E OS = Homo sapiens GN = APOE PE = 1 SV = 1 | 32 | 33 |
| 11 | Fibrinogen beta chain OS = Homo sapiens GN = FGB PE = 1 | 24 | 41 |
| | SV = 2 | | |
| 13 | Ig lambda-2 chain C regions OS = Homo sapiens GN = IGLC2 | 33 | 38 |
| | PE = 1 SV = 1 | | |
| 26 | Vinculin OS = Homo sapiens GN = VCL PE = 1 SV = 4 | 33 | 22 |
| 22 | Apolipoprotein A-I OS = Homo sapiens GN = APOA1 PE = 1 | 16 | 29 |
| | SV = 1 | | |
| 25 | Keratin, type II cytoskeletal 1 OS = Homo sapiens GN = KRT1 | 29 | 24 |
| | PE = 1 SV = 6 | | |
| 21 | Serum albumin OS = Homo sapiens GN = ALB PE = 1 SV = 2 | 18 | 30 |
| 24 | Fibrinogen gamma chain OS = Homo sapiens GN = FGG PE = 2 | 17 | 25 |
| | SV = 1 | | |
| 34 | Serum deprivation-response protein OS = Homo sapiens | 15 | 16 |
| | GN = SDPR PE = 1 SV = 3 | | |
| 27 | Immunoglobulin lambda-like polypeptide 5 OS = Homo sapiens | 17 | 21 |
| | GN = IGLL5 PE = 2 SV = 2 | | |
| 30 | Thrombospondin-1 OS = Homo sapiens GN = THBS1 PE = 1 | 24 | 18 |
| | SV = 2 | | |
| 49 | Keratin, type I cytoskeletal 9 OS = Homo sapiens GN = KRT9 | 25 | 11 |
| | PE = 1 SV = 3 | | |
| 42 | Glyceraldehyde-3-phosphate dehydrogenase OS = Homo | 15 | 12 |
| | sapiens GN = GAPDH PE = 1 SV = 3 | | |
| 48 | Ig gamma-2 chain C region OS = Homo sapiens GN = IGHG2 | 10 | 11 |
| | PE = 1 SV = 2 | | |
| 28 | Myosin light polypeptide 6 OS = Homo sapiens GN = MYL6 | 18 | 19 |
| | PE = 2 SV = 1 | | |
| 70 | Complement C1r subcomponent OS = Homo sapiens GN = C1R | 8 | 7 |
| | PE = 1 SV = 2 | | |
| 33 | Isoform 2 of Clusterin OS = Homo sapiens GN = CLU | 13 | 16 |
| 29 | Myosin regulatory light chain 12A OS = Homo sapiens | 20 | 18 |
| | GN = MYL12A PE = 4 SV = 1 | | |
| 47 | Gelsolin OS = Homo sapiens GN = GSN PE = 2 SV = 1 | 19 | 11 |
| 46 | Tubulin beta-1 chain OS = Homo sapiens GN = TUBB1 PE = 1 | 17 | 12 |
| | SV = 1 | | |
| 37 | Vitamin K-dependent protein S OS = Homo sapiens | 13 | 15 |
| | GN = PROS1 PE = 1 SV = 1 | | |
| 32 | Integrin-linked protein kinase OS = Homo sapiens GN = ILK | 10 | 17 |
| | PE = 1 SV = 2 | | |
| 81 | C4b-binding protein alpha chain OS = Homo sapiens | 9 | 6 |
| | GN = C4BPA PE = 1 SV = 2 | | |
| 40 | Ras suppressor protein 1 OS = Homo sapiens GN = RSU1 PE = 1 | 10 | 14 |
| | SV = 3 | | |
| 98 | Protein S100-A9 OS = Homo sapiens GN = S100A9 PE = 1 SV = 1 | 17 | 5 |
| 44 | Integrin alpha-IIb OS = Homo sapiens GN = ITGA2B PE = 1 | 18 | 12 |
| | SV = 3 | | |
| 41 | Heat shock cognate 71 kDa protein OS = Homo sapiens | 16 | 13 |
| | GN = HSPA8 PE = 2 SV = 1 | | |
| 43 | Ig kappa chain V-III region HIC OS = Homo sapiens PE = 2 | 16 | 12 |
| | SV = 2 | | |
| 69 | Collagen alpha-1(I) chain OS = Homo sapiens GN = COL1A1 | 5 | 7 |
| | PE = 1 SV = 5 | | |
| 53 | Galectin-3-binding protein OS = Homo sapiens | 15 | 10 |
| | GN = LGALS3BP PE = 1 SV = 1 | | |
| 35 | Isoform 2 of Haptoglobin-related protein OS = Homo sapiens | 12 | 15 |
| | GN = HPR | | |
| 50 | Tropomyosin alpha-4 chain OS = Homo sapiens GN = TPM4 | 10 | 11 |
| | PE = 1 SV = 3 | | |
| 38 | Alpha-actinin-1 OS = Homo sapiens GN = ACTN1 PE = 2 SV = 1 | 14 | 14 |
| 31 | ATP synthase subunit beta, mitochondrial OS = Homo sapiens | 11 | 17 |
| | GN = ATP5B PE = 1 SV = 3 | | |
| 45 | Moesin OS = Homo sapiens GN = MSN PE = 1 SV = 3 | 11 | 12 |
| 90 | Ficolin-3 OS = Homo sapiens GN = FCN3 PE = 1 SV = 2 | 11 | 5 |
| 39 | Complement C4-B OS = Homo sapiens GN = C4B PE = 1 SV = 2 | 11 | 14 |
| 84 | Isoform 2 of Fermitin family homolog 3 OS = Homo sapiens | 12 | 6 |
| | GN = FERMT3 | | |
| 56 | Ig kappa chain V-IV region JI OS = Homo sapiens PE = 4 SV = 1 | 8 | 9 |
| 36 | Transgelin-2 OS = Homo sapiens GN = TAGLN2 PE = 1 SV = 3 | 6 | 15 |
| 76 | Tubulin alpha-1C chain OS = Homo sapiens GN = TUBA1C | 8 | 7 |
| | PE = 2 SV = 1 | | |
| 82 | Fructose-bisphosphate aldolase OS = Homo sapiens | 12 | 6 |
| | GN = ALDOA PE = 3 SV = 1 | | |
| 65 | Keratin, type I cytoskeletal 10 OS = Homo sapiens GN = KRT10 | 8 | 8 |
| | PE = 1 SV = 6 | | |
| 54 | Heat shock protein beta-1 OS = Homo sapiens GN = HSPB1 | 4 | 10 |
| | PE = 1 SV = 2 | | |
| 62 | Ig heavy chain V-III region VH26 OS = Homo sapiens PE = 1 | 8 | 8 |
| | SV = 1 | | |
| 95 | Isoform 2 of Apolipoprotein L1 OS = Homo sapiens | 7 | 5 |
| | GN = APOL1 | | |
| 102 | Apolipoprotein D OS = Homo sapiens GN = APOD PE = 1 SV = 1 | 4 | 5 |
| 68 | ATP synthase subunit O, mitochondrial OS = Homo sapiens | 5 | 7 |
| | GN = ATP5O PE = 1 SV = 1 | | |
| 57 | Isoform 3 of Ras-related protein Rap-1b OS = Homo sapiens | 5 | 9 |
| | GN = RAP1B | | |
| 79 | Ig heavy chain V-III region BRO OS = Homo sapiens PE = 1 | 8 | 7 |
| | SV = 1 | | |
| 61 | Ig heavy chain V-III region GAL OS = Homo sapiens PE = 1 | 8 | 8 |
| | SV = 1 | | |
| 101 | Ig kappa chain V-I region HK101 (Fragment) OS = Homo | 1 | 5 |
| | sapiens PE = 4 SV = 1 | | |
| 71 | HLA class I histocompatibility antigen, A-25 alpha chain | 8 | 7 |
| | OS = Homo sapiens GN = HLA-A PE = 1 SV = 1 | | |
| 51 | Alpha-synuclein OS = Homo sapiens GN = SNCA PE = 2 SV = 1 | 4 | 10 |
| 72 | Ig alpha-1 chain C region OS = Homo sapiens GN = IGHA1 | 7 | 7 |
| | PE = 1 SV = 2 | | |
| 63 | Ig kappa chain V-III region VG (Fragment) OS = Homo sapiens | 7 | 8 |
| | PE = 1 SV = 1 | | |
| 58 | Malate dehydrogenase, mitochondrial OS = Homo sapiens | 6 | 9 |
| | GN = MDH2 PE = 1 SV = 3 | | |
| 96 | Isoform 2 of Spectrin alpha chain, erythrocytic 1 OS = Homo | 6 | 5 |
| | sapiens GN = SPTA1 | | |
| 128 | Isoform Er11 of Ankyrin-1 OS = Homo sapiens GN = ANK1 | 8 | 3 |
| 64 | Isoform Long of Erythrocyte membrane protein band 4.2 | 6 | 8 |
| | OS = Homo sapiens GN = EPB42 | | |
| 94 | Isoform 2 of Adenylyl cyclase-associated protein 1 OS = Homo | 6 | 5 |
| | sapiens GN = CAP1 | | |
| 104 | Apolipoprotein C-IV OS = Homo sapiens GN = APOC4 PE = 1 | 5 | 5 |
| | SV = 1 | | |
| 59 | 60 kDa heat shock protein, mitochondrial OS = Homo sapiens | 3 | 8 |
| | GN = HSPD1 PE = 1 SV = 2 | | |
| 52 | Complement C3 OS = Homo sapiens GN = C3 PE = 1 SV = 2 | 3 | 10 |
| 85 | Syntaxin-binding protein 2 OS = Homo sapiens GN = STXBP2 | 4 | 6 |
| | PE = 2 SV = 1 | | |
| 86 | Beta-2-microglobulin form pI 5.3 (Fragment) OS = Homo | 9 | 6 |
| | sapiens GN = B2M PE = 2 SV = 1 | | |
| 92 | Isocitrate dehydrogenase [NADP], mitochondrial OS = Homo | 3 | 5 |
| | sapiens GN = IDH2 PE = 1 SV = 2 | | |
| 74 | Profilin-1 OS = Homo sapiens GN = PFN1 PE = 1 SV = 2 | 7 | 7 |
| 77 | Tubulin beta chain OS = Homo sapiens GN = TUBB PE = 4 | 6 | 7 |
| | SV = 1 | | |
| 114 | Isoform 4 of Superoxide dismutase [Mn], mitochondrial | 5 | 4 |
| | OS = Homo sapiens GN = SOD2 | | |
| 78 | Ig lambda chain V-IV region Hil OS = Homo sapiens PE = 1 | 6 | 7 |
| | SV = 1 | | |
| 106 | ATP synthase subunit d, mitochondrial OS = Homo sapiens | 4 | 4 |
| | GN = ATP5H PE = 1 SV = 3 | | |
| 110 | Coagulation factor VIII OS = Homo sapiens GN = F8 PE = 1 | 8 | 4 |
| | SV = 1 | | |
| 80 | ATP synthase subunit alpha, mitochondrial OS = Homo sapiens | 5 | 6 |
| | GN = ATP5A1 PE = 1 SV = 1 | | |
| 122 | Ig kappa chain V-I region HK102 (Fragment) OS = Homo | 5 | 4 |
| | sapiens GN = IGKV1-5 PE = 4 SV = 1 | | |
| 130 | IgGFc-binding protein OS = Homo sapiens GN = FCGBP PE = 1 | 7 | 3 |
| | SV = 3 | | |
| 66 | Keratin, type II cytoskeletal 2 epidermal OS = Homo sapiens | 5 | 8 |
| | GN = KRT2 PE = 1 SV = 2 | | |
| 118 | Isoform 2 of Heparanase OS = Homo sapiens GN = HPSE | 4 | 4 |
| 152 | Isoform 2 of Tropomyosin alpha-3 chain OS = Homo sapiens | 4 | 2 |
| | GN = TPM3 | | |
| 87 | Histone H2A type 1-D OS = Homo sapiens GN = HIST1H2AD | 7 | 6 |
| | PE = 1 SV = 2 | | |
| 112 | Ig lambda chain V-III region LOI OS = Homo sapiens PE = 1 | 2 | 4 |
| | SV = 1 | | |
| 55 | Complement factor H OS = Homo sapiens GN = CFH PE = 1 | 0 | 9 |
| | SV = 4 | | |
| 73 | Pleckstrin OS = Homo sapiens GN = PLEK PE = 1 SV = 3 | 6 | 7 |
| 182 | Ig heavy chain V-I region V35 OS = Homo sapiens PE = 1 SV = 1 | 1 | 2 |
| 105 | 14-3-3 protein zeta/delta (Fragment) OS = Homo sapiens | 4 | 4 |
| | GN = YWHAZ PE = 2 SV = 1 | | |
| 91 | Hemoglobin subunit delta OS = Homo sapiens GN = HBD PE = 1 | 5 | 5 |
| | SV = 2 | | |
| 113 | Integrin beta-3 OS = Homo sapiens GN = ITGB3 PE = 1 SV = 2 | 8 | 4 |
| 100 | Lipopolysaccharide-binding protein OS = Homo sapiens | 6 | 5 |
| | GN = LBP PE = 1 SV = 3 | | |
| 120 | Serum amyloid A-4 protein OS = Homo sapiens GN = SAA4 | 4 | 4 |
| | PE = 1 SV = 2 | | |
| 103 | Platelet basic protein OS = Homo sapiens GN = PPBP PE = 1 | 5 | 5 |
| | SV = 3 | | |
| 121 | Cytochrome c oxidase subunit 5A, mitochondrial OS = Homo | 4 | 4 |
| | sapiens GN = COX5A PE = 1 SV = 2 | | |
| 75 | Protein disulfide isomerase family A, member 3, isoform | 0 | 7 |
| | CRA_b OS = Homo sapiens GN = PDIA3 PE = 3 SV = 1 | | |
| 217 | Trypsin-1 OS = Homo sapiens GN = PRSS1 PE = 2 SV = 1 | 0 | 1 |
| 107 | Actin-related protein 2/3 complex subunit 3 OS = Homo | 6 | 4 |
| | sapiens GN = ARPC3 PE = 1 SV = 3 | | |
| 117 | Trypsin-3 (Fragment) OS = Homo sapiens GN = PRSS3 PE = 2 | 3 | 4 |
| | SV = 1 | | |
| 141 | Cytochrome c oxidase subunit 5B, mitochondrial OS = Homo | 3 | 3 |
| | sapiens GN = COX5B PE = 1 SV = 2 | | |
| 195 | Peptidyl-prolyl cis-trans isomerase A OS = Homo sapiens | 8 | 1 |
| | GN = PPIA PE = 1 SV = 2 | | |
| 145 | Ig heavy chain V-III region WEA OS = Homo sapiens PE = 1 | 2 | 3 |
| | SV = 1 | | |
| 125 | Ig kappa chain V-I region Wes OS = Homo sapiens PE = 1 SV = 1 | 2 | 4 |
| 207 | Coagulation factor V OS = Homo sapiens GN = F5 PE = 1 SV = 4 | 5 | 1 |
| 83 | Isoform 2 of Beta-parvin OS = Homo sapiens GN = PARVB | 2 | 6 |
| 178 | Immunoglobulin J chain (Fragment) OS = Homo sapiens | 2 | 2 |
| | GN = IGJ PE = 2 SV = 1 | | |
| 108 | Band 3 anion transport protein OS = Homo sapiens | 4 | 4 |
| | GN = SLC4A1 PE = 1 SV = 3 | | |
| 97 | Phosphatidylinositol 5-phosphate 4-kinase type-2 alpha | 3 | 5 |
| | OS = Homo sapiens GN = PIP4K2A PE = 1 SV = 2 | | |
| 163 | Coagulation factor XIII A chain OS = Homo sapiens | 6 | 2 |
| | GN = F13A1 PE = 1 SV = 4 | | |
| 60 | Histidine-rich glycoprotein OS = Homo sapiens GN = HRG | 2 | 8 |
| | PE = 1 SV = 1 | | |
| 123 | Myosin regulatory light polypeptide 9 OS = Homo sapiens | 5 | 4 |
| | GN = MYL9 PE = 1 SV = 4 | | |
| 135 | 10 kDa heat shock protein, mitochondrial OS = Homo sapiens | 2 | 3 |
| | GN = HSPE1 PE = 1 SV = 2 | | |
| 177 | Apolipoprotein A-II OS = Homo sapiens GN = APOA2 PE = 1 | 1 | 2 |
| | SV = 1 | | |
| 67 | 78 kDa glucose-regulated protein OS = Homo sapiens | 2 | 7 |
| | GN = HSPA5 PE = 1 SV = 2 | | |
| 189 | Apolipoprotein M OS = Homo sapiens GN = APOM PE = 2 SV = 1 | 2 | 2 |
| 191 | Ig kappa chain V-I region AG OS = Homo sapiens PE = 1 SV = 1 | 2 | 2 |
| 88 | Alpha-1-antitrypsin OS = Homo sapiens GN = SERPINA1 PE = 1 | 1 | 5 |
| | SV = 3 | | |
| 172 | Tropomodulin-3 OS = Homo sapiens GN = TMOD3 PE = 1 SV = 1 | 6 | 2 |
| 173 | Glutathione peroxidase 1 OS = Homo sapiens GN = GPX1 PE = 1 | 3 | 2 |
| | SV = 4 | | |
| 126 | Isoform 2 of Bridging integrator 2 OS = Homo sapiens | 2 | 3 |
| | GN = BIN2 | | |
| 119 | Ig kappa chain V-II region GM607 (Fragment) OS = Homo | 2 | 4 |
| | sapiens PE = 4 SV = 1 | | |
| 215 | Protein S100-A8 OS = Homo sapiens GN = S100A8 PE = 1 SV = 1 | 2 | 1 |
| 111 | Cofilin-1 OS = Homo sapiens GN = CFL1 PE = 1 SV = 3 | 3 | 4 |
| 131 | Multimerin-1 OS = Homo sapiens GN = MMRN1 PE = 2 SV = 1 | 4 | 3 |
| 116 | Stress-70 protein, mitochondrial OS = Homo sapiens | 4 | 4 |
| | GN = HSPA9 PE = 1 SV = 2 | | |
| 93 | Isoform 2 of ATP synthase-coupling factor 6, mitochondrial | 3 | 5 |
| | OS = Homo sapiens GN = ATP5J | | |
| 136 | Hydroxyacyl-coenzyme A dehydrogenase, mitochondrial | 3 | 3 |
| | OS = Homo sapiens GN = HADH PE = 4 SV = 1 | | |
| 137 | Complement C1q subcomponent subunit B OS = Homo sapiens | 3 | 3 |
| | GN = C1QB PE = 1 SV = 3 | | |
| 133 | Vasodilator-stimulated phosphoprotein OS = Homo sapiens | 4 | 3 |
| | GN = VASP PE = 1 SV = 3 | | |
| 115 | NADH dehydrogenase [ubiquinone] 1 beta subcomplex | 3 | 4 |
| | subunit 10 OS = Homo sapiens GN = NDUFB10 PE = 1 SV = 3 | | |
| 99 | Zyxin (Fragment) OS = Homo sapiens GN = ZYX PE = 2 SV = 1 | 2 | 5 |
| 124 | Ig gamma-4 chain C region OS = Homo sapiens GN = IGHG4 | 4 | 4 |
| | PE = 1 SV = 1 | | |
| 196 | Citrate synthase OS = Homo sapiens GN = CS PE = 2 SV = 1 | 2 | 1 |
| 89 | Apolipoprotein A-IV OS = Homo sapiens GN = APOA4 PE = 1 | 1 | 5 |
| | SV = 3 | | |
| 194 | Ig kappa chain V-I region WEA OS = Homo sapiens PE = 1 | 3 | 2 |
| | SV = 1 | | |
| 240 | Collagen alpha-3(VI) chain OS = Homo sapiens GN = COL6A3 | 5 | 0 |
| | PE = 2 SV = 1 | | |
| 109 | Brain acid soluble protein 1 OS = Homo sapiens GN = BASP1 | 3 | 4 |
| | PE = 1 SV = 2 | | |
| 161 | ATP synthase subunit gamma, mitochondrial OS = Homo | 1 | 2 |
| | sapiens GN = ATP5C1 PE = 1 SV = 1 | | |
| 143 | Single-stranded DNA-binding protein, mitochondrial | 2 | 3 |
| | OS = Homo sapiens GN = SSBP1 PE = 1 SV = 1 | | |
| 146 | Peroxiredoxin 2, isoform CRA_a OS = Homo sapiens | 3 | 3 |
| | GN = PRDX2 PE = 4 SV = 2 | | |
| 138 | Protein NipSnap homolog 3A OS = Homo sapiens | 1 | 3 |
| | GN = NIPSNAP3A PE = 1 SV = 2 | | |
| 183 | Peptidyl-prolyl cis-trans isomerase F, mitochondrial | 2 | 2 |
| | OS = Homo sapiens GN = PPIF PE = 1 SV = 1 | | |
| 147 | Thymosin beta-4 OS = Homo sapiens GN = TMSB4X PE = 1 | 2 | 3 |
| | SV = 2 | | |
| 148 | Protein disulfide-isomerase A6 OS = Homo sapiens | 4 | 3 |
| | GN = PDIA6 PE = 2 SV = 1 | | |
| 201 | Beta-2-glycoprotein 1 OS = Homo sapiens GN = APOH PE = 1 | 2 | 1 |
| | SV = 3 | | |
| 213 | F-actin-capping protein subunit alpha-1 OS = Homo sapiens | 3 | 1 |
| | GN = CAPZA1 PE = 1 SV = 3 | | |
| 157 | Inter-alpha-trypsin inhibitor heavy chain H2 OS = Homo | 1 | 2 |
| | sapiens GN = ITIH2 PE = 1 SV = 2 | | |
| 254 | Protein S100-A7 OS = Homo sapiens GN = S100A7 PE = 1 SV = 4 | 5 | 0 |
| 154 | Lactadherin short form OS = Homo sapiens GN = MFGE8 PE = 2 | 2 | 2 |
| | SV = 1 | | |
| 169 | Tripeptidyl-peptidase 1 OS = Homo sapiens GN = TPP1 PE = 1 | 3 | 2 |
| | SV = 2 | | |
| 139 | Haptoglobin OS = Homo sapiens GN = HP PE = 1 SV = 1 | 2 | 3 |
| 166 | Peroxiredoxin-6 OS = Homo sapiens GN = PRDX6 PE = 1 SV = 3 | 2 | 2 |
| 132 | Serotransferrin OS = Homo sapiens GN = TF PE = 1 SV = 3 | 2 | 3 |
| 181 | Tubulin alpha-4A chain OS = Homo sapiens GN = TUBA4A | 1 | 2 |
| | PE = 2 SV = 1 | | |
| 185 | Actin-related protein 2/3 complex subunit 2 OS = Homo | 1 | 2 |
| | sapiens GN = ARPC2 PE = 1 SV = 1 | | |
| 153 | Prohibitin-2 OS = Homo sapiens GN = PHB2 PE = 2 SV = 1 | 1 | 2 |
| 202 | L-lactate dehydrogenase B chain OS = Homo sapiens | 3 | 1 |
| | GN = LDHB PE = 1 SV = 2 | | |
| 158 | Coiled-coil-helix-coiled-coil-helix domain-containing protein | 1 | 2 |
| | 3, mitochondrial OS = Homo sapiens GN = CHCHD3 PE = 2 | | |
| | SV = 1 | | |
| 193 | Ig lambda chain V-III region SH OS = Homo sapiens PE = 1 | 1 | 2 |
| | SV = 1 | | |
| 190 | Plasminogen OS = Homo sapiens GN = PLG PE = 1 SV = 2 | 2 | 2 |
| 225 | Alpha-enolase OS = Homo sapiens GN = ENO1 PE = 1 SV = 2 | 3 | 1 |
| 224 | Ig kappa chain V-III region NG9 (Fragment) OS = Homo | 1 | 1 |
| | sapiens PE = 1 SV = 1 | | |
| 149 | Ig heavy chain V-III region BUT OS = Homo sapiens PE = 1 | 1 | 3 |
| | SV = 1 | | |
| 236 | Catalase OS = Homo sapiens GN = CAT PE = 1 SV = 3 | 4 | 0 |
| 233 | Ig lambda chain V-I region WAH OS = Homo sapiens PE = 1 | 2 | 1 |
| | SV = 1 | | |
| 243 | Histone H4 OS = Homo sapiens GN = HIST1H4A PE = 1 SV = 2 | 3 | 0 |
| 250 | Keratin, type I cytoskeletal 14 OS = Homo sapiens GN = KRT14 | 5 | 0 |
| | PE = 1 SV = 4 | | |
| 156 | Myristoylated alanine-rich C-kinase substrate OS = Homo | 2 | 2 |
| | sapiens GN = MARCKS PE = 1 SV = 4 | | |
| 144 | Histidine triad nucleotide-binding protein 2, mitochondrial | 2 | 3 |
| | OS = Homo sapiens GN = HINT2 PE = 1 SV = 1 | | |
| 140 | 2,4-dienoyl-CoA reductase, mitochondrial OS = Homo sapiens | 1 | 3 |
| | GN = DECR1 PE = 2 SV = 1 | | |
| 187 | Ras-related protein Rab-10 OS = Homo sapiens GN = RAB10 | 3 | 2 |
| | PE = 1 SV = 1 | | |
| 203 | Protein 4.1 OS = Homo sapiens GN = EPB41 PE = 2 SV = 1 | 1 | 1 |
| 176 | Charged multivesicular body protein 4b OS = Homo sapiens | 1 | 2 |
| | GN = CHMP4B PE = 1 SV = 1 | | |
| 192 | ATP synthase subunit e, mitochondrial OS = Homo sapiens | 1 | 2 |
| | GN = ATP5I PE = 1 SV = 2 | | |
| 218 | Serum paraoxonase/arylesterase 1 OS = Homo sapiens | 2 | 1 |
| | GN = PON1 PE = 2 SV = 1 | | |
| 219 | Ig kappa chain V-I region Mev OS = Homo sapiens PE = 1 | 1 | 1 |
| | SV = 1 | | |
| 210 | Alpha-1-acid glycoprotein 2 OS = Homo sapiens GN = ORM2 | 1 | 1 |
| | PE = 1 SV = 2 | | |
| 223 | Collagen alpha-1(III) chain OS = Homo sapiens GN = COL3A1 | 2 | 1 |
| | PE = 1 SV = 4 | | |
| 127 | Isoform 2 of Thioredoxin-dependent peroxide reductase, | 1 | 3 |
| | mitochondrial OS = Homo sapiens GN = PRDX3 | | |
| 150 | Ig kappa chain V-I region Ni OS = Homo sapiens PE = 1 SV = 1 | 1 | 3 |
| 234 | Ig heavy chain V-III region TRO OS = Homo sapiens PE = 1 | 1 | 1 |
| | SV = 1 | | |
| 242 | Platelet glycoprotein Ib alpha chain OS = Homo sapiens | 1 | 0 |
| | GN = GP1BA PE = 1 SV = 2 | | |
| 256 | Transitional endoplasmic reticulum ATPase OS = Homo | 4 | 0 |
| | sapiens GN = VCP PE = 1 SV = 4 | | |
| 186 | Cytochrome c oxidase subunit 7A2, mitochondrial OS = Homo | 2 | 2 |
| | sapiens GN = COX7A2 PE = 1 SV = 1 | | |
| 208 | Tropomyosin alpha-1 chain OS = Homo sapiens GN = TPM1 | 2 | 1 |
| | PE = 2 SV = 1 | | |
| 171 | Latent-transforming growth factor beta-binding protein 1 | 1 | 2 |
| | OS = Homo sapiens GN = LTBP1 PE = 2 SV = 1 | | |
| 205 | Cytochrome c oxidase subunit 6C OS = Homo sapiens | 1 | 1 |
| | GN = COX6C PE = 1 SV = 2 | | |
| 184 | Isoform 2 of Clathrin heavy chain 1 OS = Homo sapiens | 1 | 2 |
| | GN = CLTC | | |
| 214 | Cytochrome c oxidase subunit 4 isoform 1, mitochondrial | 2 | 1 |
| | OS = Homo sapiens GN = COX4I1 PE = 1 SV = 1 | | |
| 226 | HLA class I histocompatibility antigen, B-35 alpha chain | 1 | 1 |
| | OS = Homo sapiens GN = HLA-B PE = 1 SV = 1 | | |
| 229 | Cytochrome b-c1 complex subunit 7 OS = Homo sapiens | 3 | 1 |
| | GN = UQCRB PE = 1 SV = 2 | | |
| 227 | Actin-related protein 2/3 complex subunit 5 OS = Homo | 1 | 1 |
| | sapiens GN = ARPC5 PE = 1 SV = 3 | | |
| 228 | Chloride intracellular channel protein 1 OS = Homo sapiens | 1 | 1 |
| | GN = CLIC1 PE = 1 SV = 4 | | |
| 160 | Thiosulfate sulfurtransferase OS = Homo sapiens GN = TST | 1 | 2 |
| | PE = 1 SV = 4 | | |
| 170 | Golgi-associated plant pathogenesis-related protein 1 | 1 | 2 |
| | OS = Homo sapiens GN = GLIPR2 PE = 1 SV = 3 | | |
| 175 | Alpha-2-HS-glycoprotein OS = Homo sapiens GN = AHSG | 1 | 2 |
| | PE = 2 SV = 1 | | |
| 134 | Cysteine and glycine-rich protein 1 OS = Homo sapiens | 1 | 3 |
| | GN = CSRP1 PE = 1 SV = 3 | | |
| 220 | Ras-related protein Rab-8A OS = Homo sapiens GN = RAB8A | 0 | 1 |
| | PE = 1 SV = 1 | | |
| 151 | Glutathione S-transferase kappa 1 OS = Homo sapiens | 0 | 2 |
| | GN = GSTK1 PE = 2 SV = 1 | | |
| 129 | Rho GDP-dissociation inhibitor 2 OS = Homo sapiens | 0 | 3 |
| | GN = ARHGDIB PE = 1 SV = 3 | | |
| 257 | Trifunctional enzyme subunit alpha, mitochondrial OS = Homo | 3 | 0 |
| | sapiens GN = HADHA PE = 1 SV = 2 | | |
| 165 | Carbonic anhydrase 1 OS = Homo sapiens GN = CA1 PE = 1 | 1 | 2 |
| | SV = 2 | | |
| 167 | Serum amyloid A protein OS = Homo sapiens GN = SAA1 PE = 3 | 1 | 2 |
| | SV = 1 | | |
| 168 | Transthyretin OS = Homo sapiens GN = TTR PE = 1 SV = 1 | 1 | 2 |
| 231 | Pyruvate kinase PKM OS = Homo sapiens GN = PKM PE = 1 | 2 | 1 |
| | SV = 4 | | |
| 198 | Coronin OS = Homo sapiens GN = CORO1C PE = 2 SV = 1 | 1 | 1 |
| 232 | Tubulin beta-4B chain OS = Homo sapiens GN = TUBB4B | 2 | 1 |
| | PE = 1 SV = 1 | | |
| 212 | Coronin-1A OS = Homo sapiens GN = CORO1A PE = 1 SV = 4 | 1 | 1 |
| 230 | NAD-dependent malic enzyme, mitochondrial OS = Homo | 1 | 1 |
| | sapiens GN = ME2 PE = 1 SV = 1 | | |
| 209 | Peroxisomal multifunctional enzyme type 2 OS = Homo | 1 | 1 |
| | sapiens GN = HSD17B4 PE = 2 SV = 3 | | |
| 199 | ADP/ATP translocase 2 OS = Homo sapiens GN = SLC25A5 | 1 | 1 |
| | PE = 1 SV = 7 | | |
| 235 | Isoform 2 of Triosephosphate isomerase OS = Homo sapiens | 2 | 1 |
| | GN = TPI1 | | |
| 211 | Ig kappa chain V-II region FR OS = Homo sapiens PE = 1 SV = 1 | 1 | 1 |
| 237 | HLA class I histocompatibility antigen, A-69 alpha chain | 2 | 0 |
| | OS = Homo sapiens GN = HLA-A PE = 1 SV = 2 | | |
| 238 | Clathrin light chain A OS = Homo sapiens GN = CLTA PE = 2 | 1 | 0 |
| | SV = 1 | | |
| 239 | Elongation factor 1-alpha 1 OS = Homo sapiens GN = EEF1A1 | 2 | 0 |
| | PE = 1 SV = 1 | | |
| 188 | Prohibitin OS = Homo sapiens GN = PHB PE = 1 SV = 1 | 0 | 2 |
| 216 | Ig lambda chain V-V region DEL OS = Homo sapiens PE = 1 | 0 | 1 |
| | SV = 1 | | |
| 241 | Ig heavy chain V-III region KOL OS = Homo sapiens PE = 1 | 1 | 0 |
| | SV = 1 | | |
| 222 | Ras-related protein Rab-27B OS = Homo sapiens | 0 | 1 |
| | GN = RAB27B PE = 1 SV = 4 | | |
| 245 | Erythrocyte band 7 integral membrane protein OS = Homo | 2 | 0 |
| | sapiens GN = STOM PE = 2 SV = 1 | | |
| 155 | Protein disulfide-isomerase A5 OS = Homo sapiens | 0 | 2 |
| | GN = PDIA5 PE = 1 SV = 1 | | |
| 159 | Isoform 3 of GTP: AMP phosphotransferase AK3, | 0 | 2 |
| | mitochondrial OS = Homo sapiens GN = AK3 | | |
| 162 | Isoform 2 of Tumor protein D54 OS = Homo sapiens | 0 | 2 |
| | GN = TPD52L2 | | |
| 142 | PDZ and LIM domain protein 1 OS = Homo sapiens | 0 | 3 |
| | GN = PDLIM1 PE = 1 SV = 4 | | |
| 253 | Oncoprotein-induced transcript 3 protein OS = Homo sapiens | 2 | 0 |
| | GN = OIT3 PE = 1 SV = 2 | | |
| 197 | Peptidyl-prolyl cis-trans isomerase B OS = Homo sapiens | 0 | 1 |
| | GN = PPIB PE = 1 SV = 2 | | |
| 251 | Keratin, type II cytoskeletal 5 OS = Homo sapiens GN = KRT5 | 1 | 0 |
| | PE = 1 SV = 3 | | |
| 206 | C6orf25 protein OS = Homo sapiens GN = C6orf25 PE = 2 SV = 1 | 0 | 1 |
| 252 | Keratin, type II cytoskeletal 6A OS = Homo sapiens | 2 | 0 |
| | GN = KRT6A PE = 1 SV = 3 | | |
| 164 | Aconitate hydratase, mitochondrial OS = Homo sapiens | 0 | 2 |
| | GN = ACO2 PE = 2 SV = 1 | | |
| 174 | Isoform 2 of Ficolin-2 OS = Homo sapiens GN = FCN2 | 0 | 2 |
| 244 | Actin-related protein 3 OS = Homo sapiens GN = ACTR3 PE = 1 | 2 | 0 |
| | SV = 3 | | |
| 258 | Ubiquitin OS = Homo sapiens GN = UBB PE = 2 SV = 1 | 2 | 0 |
| 179 | Adenylate kinase 2, mitochondrial OS = Homo sapiens | 0 | 2 |
| | GN = AK2 PE = 2 SV = 1 | | |
| 180 | Cytochrome c (Fragment) OS = Homo sapiens GN = CYCS | 0 | 2 |
| | PE = 2 SV = 1 | | |
| 200 | Isoform 2 of Prostaglandin G/H synthase 1 OS = Homo sapiens | 0 | 1 |
| | GN = PTGS1 | | |
| 204 | Caldesmon OS = Homo sapiens GN = CALD1 PE = 2 SV = 1 | 0 | 1 |
| 249 | Isoform 2 of Utrophin OS = Homo sapiens GN = UTRN | 1 | 0 |
| 246 | Isoform 2 of Electron transfer flavoprotein subunit beta | 1 | 0 |
| | OS = Homo sapiens GN = ETFB | | |
| 247 | Isoform 2 of Mannan-binding lectin serine protease 1 | 1 | 0 |
| | OS = Homo sapiens GN = MASP1 | | |
| 248 | Isoform 2 of Platelet glycoprotein Ib beta chain OS = Homo | 1 | 0 |
| | sapiens GN = GP1BB | | |
| 259 | WD repeat-containing protein 1 OS = Homo sapiens | 1 | 0 |
| | GN = WDR1 PE = 1 SV = 4 | | |
| 255 | Transferrin receptor protein 1, serum form OS = Homo sapiens | 1 | 0 |
| | GN = TFRC PE = 2 SV = 1 | | |
| 221 | T-complex protein 1 subunit epsilon OS = Homo sapiens | 0 | 1 |
| | GN = CCT5 PE = 2 SV = 1 | | |

### Example 12: FXYD2 Enriched Exosome Platform as a Diagnostic Assay for Monitoring Transplanted Islet Cells in a Type 1 Diabetic Patient Undergoing Single Donor Islet Cell Transplantation

To understand if the transplant islet specific exosomes platform can be translated to the human clinical setting, plasma EVs were analyzed from a type I diabetic patient undergoing islet transplantation via portal vein infusion of single donor allogeneic islets. This example demonstrates that islet exosomes show differences in exosome profiles in type I diabetic patients as compared to normal controls. FXYD2 has two isoforms, γA and γB, which are expressed on the surface of islet beta cell exosomes. FXYD2 γA antibody was used as a marker to enrich islet beta cell exosomes from patient blood. It was found that insulin containing exosomes were enriched as compared to non-FXYD2 exosomes.

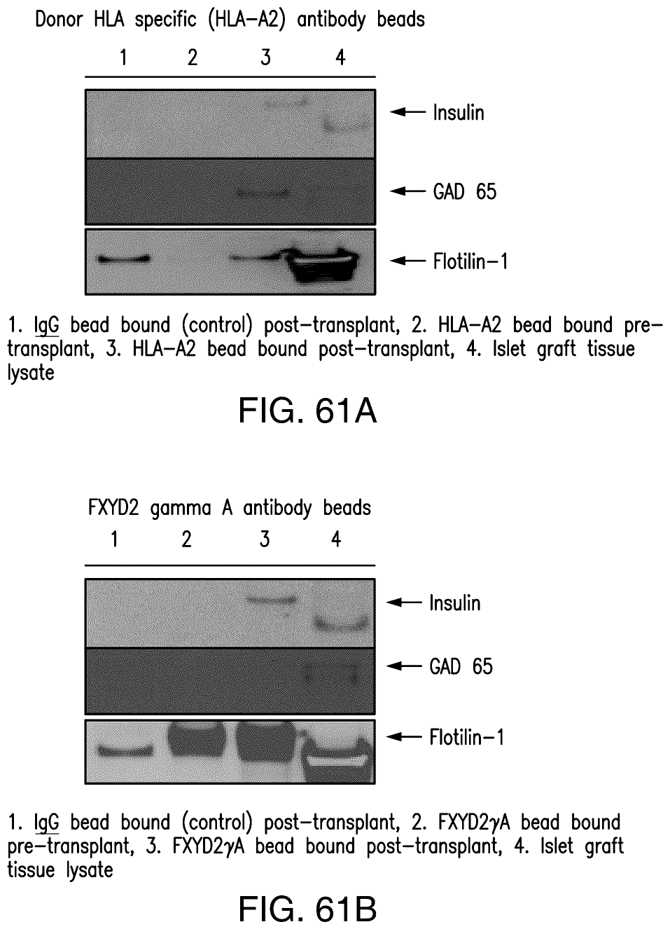

FIG. 61 evaluations transplant islet exosomes in a type 1 diabetic patient undergoing single donor islet cell transplantation. A recipient diabetic patient (i.e., without native insulin production) underwent islet cell transplantation (donor HLA-A2 positive, recipient HLA-A2 negative) and maintained normoglycemia post-transplantation. Pre-transplant and post-transplant plasma sample from the recipient was analyzed first using anti-HLA-A2 antibody beads to purify transplant islet exosomes (FIG. 61A). Compared to the pre-transplant sample, the post-transplant HLA-A2 bound exosomes contained insulin protein as part of its cargo, along with GAD65 (islet beta cell protein). IgG antibody (mouse) beads in the post-transplant sample were used as negative control, and therefore did not show insulin or GAD65 expression. Flotillin-1 is a canonical exosome marker protein. In the same recipient patient, it was studied whether FXYD2γA antibody beads would also enrich for insulin expressing exosomes, validating that this protein surface marker can be used as a methodology to enrich for islet beta cell exosomes. FIG. 61B demonstrates that FXYD2γA can be used as a marker to enrich for beta cell exosomes, as the bead bound pre-transplant recipient sample did not show insulin expression while the post-transplant did show insulin expression. IgG antibody (rabbit) beads in the post-transplant plasma sample were used as a control. This experiment demonstrates that compared to whole plasma exosome analysis or non-specific exosome binding (IgG control), FXYD2γA antibody can serve to enrich for islet beta cell exosomes.

### Example 13: Quantitative Changes in Transplant Tissue Specific Exosome Number as a Biomarker to Monitor Kidney Transplant Rejection

Donor kidney specific exosomes can be assessed from urine samples in patients undergoing living donor renal transplantation. A donor HLA profile can be compared to a recipient HLA profile, and any HLA proteins specific for the donor or recipient can be used to identify or purify exosomes specific to either the donor or recipient.

Donor renal specific exosomes can be assessed utilizing antibodies directed to HLA proteins specific for the donor. Recipient T-cell specific exosome fraction can be assessed, for example, using anti-CD3 antibodies. Unbound donor-HLA and unbound CD urine exosomes positive for B-cell surface markers can also be assessed.

The donor renal specific exosomes and recipient T-cell and B-cell specific exosomes can be assessed to determine if there is a change in the size and/or number of the separate pools of exosomes. The results can be compared to a normal subject; a reference as detected before clinical signs of the return of the disease treated by the transplantation; or a reference as detected before the clinical onset of rejection.

### Example 14: Plasma Transplant Islet Exosome Signal Heralds Acute Rejection Prior to Detection of Hyperglycemia

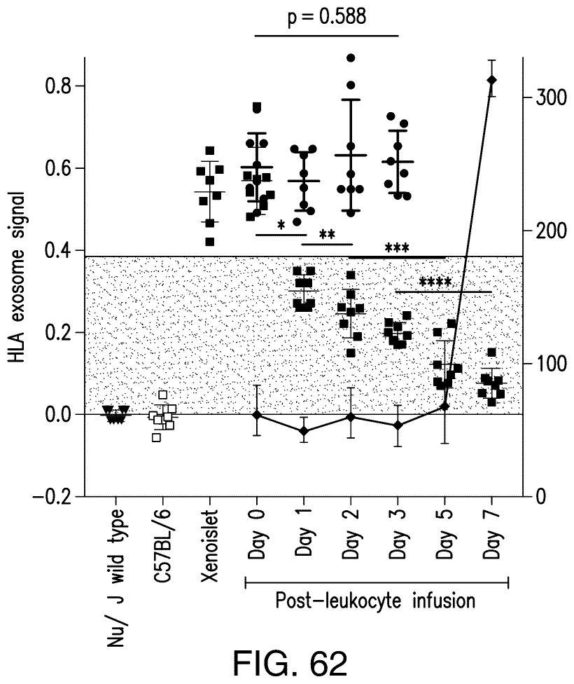

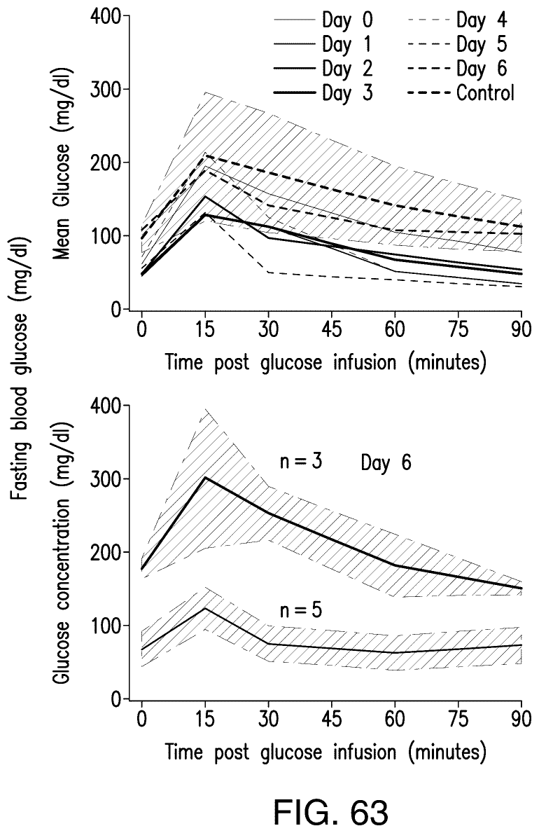

Biomarker use of the transplant exosome platform was investigated by performing a comparative analysis of the kinetics of the HLA exosome signal versus blood glucose monitoring during the evolution of rejection in the xenoislet model. In two independent experiments, donor sensitized leukocytes from syngeneic wild type animals were infused into normoglycemic xenoislet recipients, and recipient plasma and islet graft were procured and analyzed at the following time points after leukocyte infusion (n=8 animals per time point): day 0 (4 hours), 1, 2, 3, 5, and 7. Whereas the fasting blood glucose remained normal during the first 6 days after infusion of donor sensitized leukocytes, the HLA exosome signal significantly decreased by day 1 (FIG. 62), compared to the signal from day 0, xenoislet, and placebo infused time point control recipients. Further, over the next 5 days of the follow-up, the HLA exosome signal continued to decrease (FIG. 62), whereas the total plasma exosome quantity was unchanged (p=0.62 by one way ANOVA; data not shown). The HLA exosome signal change was significantly different between the time points tested, and suggested a pattern of exponential decay in HLA exosome quantity (FIG. 62). In the xenoislet animals receiving placebo infusion, there was no difference in the HLA exosome signal between time points. Daily intraperitoneal glucose tolerance tests were also performed on all animals and measured recipient plasma human specific C-peptide levels for the time points tested to compare to the HLA exosome signal. Glucose tolerance tests remained normal through day 5, and showed only mild impairment in glucose disposal on day 6 (FIG. 63), one day before the fasting blood glucose became elevated. Stimulated human C-peptide levels also remained within normal range through day 5 (data not shown).

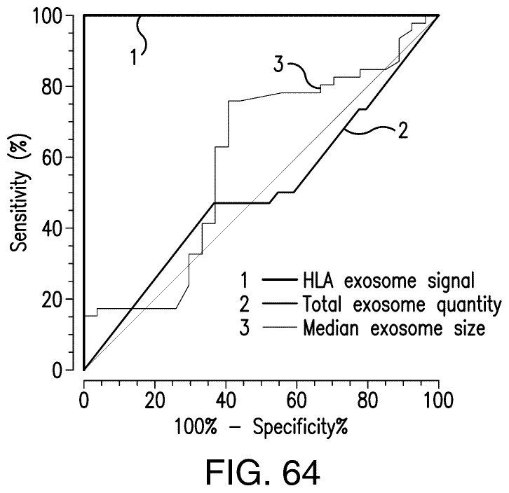

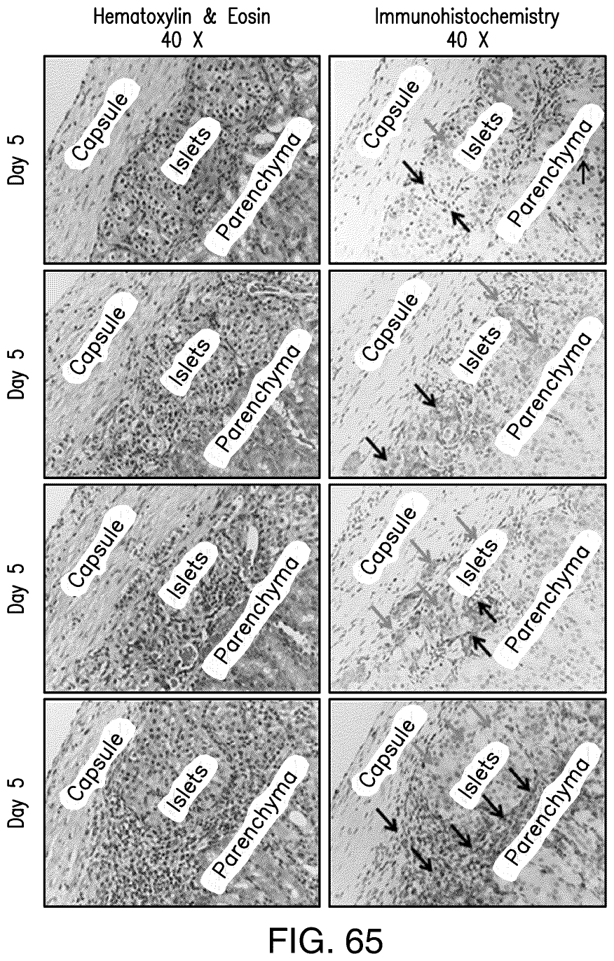

Given the significant drop in HLA exosome signal by day 1 time point, a receiver operating characteristic (ROC) curve was generated to understand its diagnostic potential to discriminate between no rejection (xenoislet, N-xeno, and day 0 time points, n=64) versus rejection (day 1 or greater time points, n=40). This showed an area under curve of 0.963, with sensitivity and specificity of 100% for signal threshold cut-off of 0.36 (FIG. 64). To further correlate these findings with the acute rejection process at the tissue level, islet graft histology was performed (FIG. 65). Day 1 histology showed few infiltrating CD3 positive T cells around viable islet clusters, although the HLA exosome signal was already significantly lower by this time point. By day 5, progressive T cell infiltration with islet destruction was evident on histology, when fasting glucose, glucose tolerance test, and human C-peptide were still within normal range. Without being bound by theory, these findings support that unlike glucose monitoring, the decay in plasma HLA exosome signal correlates with early changes in the acute rejection process, when there is minimal T cell infiltrate without islet destruction. Collectively, these data demonstrate that in the xenoislet model, transplant islet exosome profiling is a more accurate and time sensitive non-invasive biomarker of acute rejection than current standards such as fasting glucose, glucose tolerance test, and human C-peptide analysis.

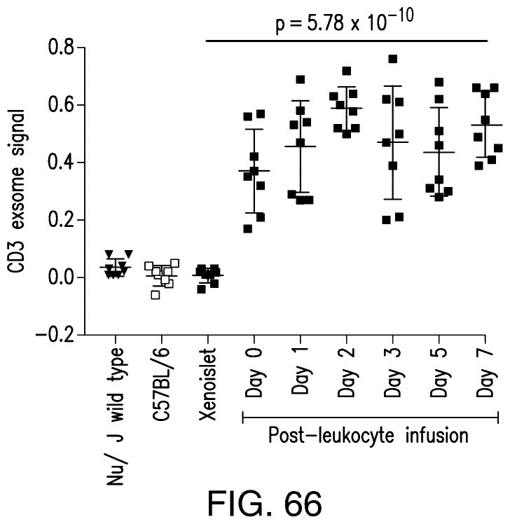

Next, it was hypothesized that acute rejection of islets could also lead to sustained elevation in recipient plasma T cell exosome signal, as stimulated xenoreactive T cells could release a steady pool of exosomes into the plasma. A combined analysis of donor MHC specific exosome signal and recipient T cell exosome characterization may further increase the accuracy of the proposed biomarker platform. Compared to naive wild type strain matched (Nu/J), 3^{rd} party C57/B16, and xenoislet controls, acute rejection animals from day 0 showed a quantifiable CD3 exosome signal in the recipient plasma, representing T cell contribution into the recipient plasma exosome pool (FIG. 66). The CD3 exosome signal remained elevated through day 7, during the entire rejection process. Taken together, this is evidence that a combined plasma tissue specific exosome profiling (donor HLA, recipient T cell and B cell) can provide a time sensitive non-invasive window into the rejection specific interactions between the transplant tissue and the recipient immune system.

### REFERENCES

1. Gieser, G., Harigaya, H., Colangelo, P. M. & Burckart, G. Biomarkers in solid organ transplantation. Clin Pharmacol Ther. 90, 217-220 (2011).
2. Mas, V. R., Mueller, T. F., Archer, K. J. & Maluf, D. G. Identifying biomarkers as diagnostic tools in kidney transplantation. Expert Rev Mol Diagn. 11(2), 183-196 (2011).
3. Fleissner, F., Goerzig, Y., Haverich, A. & Thum, T. Microvesicles as novel biomarkers and therapeutic targets in transplantation medicine. Am J Transplant. 12(2), 289-297 (2012).
4. Farid, W. R. M. et al. Hepatocyte-derived microRNAs as serum biomarkers of hepatic injury and rejection after liver transplantation. Liver Transpl. 18, 290-297 (2012).
5. Shan, J., Feng, L., Luo, L., Wu, W., Li, C., Li, S. & Li, Y. MicroRNAs: potential biomarker in organ transplantation. Transpl. Immunol. 24, 210 (2011).
6. Roedder, S., Vitalone, M., Khatri, P. & Sarwal, M. M. Biomarkers in solid organ transplantation: establishing personalized transplantation medicine. Genome Med. 3(6), 37 (2011).
7. Denise, J., Lo, B. K. & Allan, D. K. Biomarkers for kidney transplant rejection. Nature Reviews Nephrology. 10, 215-225 (2014).
8. Matsumoto, S. et al. Circulating p53-responsive microRNAs are predictive indicators of heart failure after acute myocardial infarction. Circ Res. 113(3):322-326 (2013).
9. Mathivanan, S., Ji, H. & Simpson, R. J. Exosomes: Extracellular organelles important in intercellular communication. J Proteomics. 73, 1907-1920 (2010).
10. Valadi, H. et al. Exosome-mediated transfer of mRNAs and microRNAs is a novel mechanism of genetic exchange between cells. Nat. Cell. Biol. 9, 654-659 (2007).
11. Akkaraju, A. & Rodriguez-Boulan, E. Itinerant exosomes: emerging roles in cell and tissue polarity. Trends in cell biology. 18(5), 199-209 (2008).
12. Turchinovich, A., Samatov, T. R., Tonevitsky, A. G. & Burwinkel, B. Circulating miRNAs: cell-cell communication function? Front Genet. 4,119 (2013).
13. Julich, H., Willms, A., Lukacs-Kornek, V. & Kornek, M. Extracellular vesicle profiling and their use as potential disease specific biomarker. Front Immunol. 5, 413 (2014).
14. Donald, J. J. & Josip, B. Biomarker discovery: tissues versus fluids versus both. Expert Rev Mol Diagn. 7(5), 473-475 (2007).
15. Hald, J. et al. Pancreatic islet and progenitor cell surface markers with cell sorting potential. Diabetologia. 55(1), 154-65 (2012).
16. Arystarkhova, E. et al. Hyperplasia of pancreatic beta cells and improved glucose tolerance in mice deficient in the FXYD2 subunit of Na,K-ATPase. J Biol Chem. 288(10), 7077-85 (2013).
17. Wang, F. et al. FXYD2, a γ subunit of Na⁺, K⁺-ATPase, maintains persistent mechanical allodynia induced by inflammation. Cell Res. 25(3), 318-34 (2015.
18. Flamez, D. et al. A genomic-based approach identifies FXYD domain containing ion transport regulator 2 (FXYD2) gamma a as a pancreatic beta cell-specific biomarker. Diabetologia. 53(7), 1372-83(2010).
19. Jopling, C. Liver-specific microRNA-122: Biogenesis and function. RNA Biology. 9(2), 137-142 (2012).
20. Thakral, S. & Ghoshal, K. miR-122 is a unique molecule with great potential in diagnosis, prognosis of liver disease, and therapy both as miRNA mimic and antimir. Curr Gene Ther. 15(2), 142-150 (2015).
21. Tsai, W. C. et al. microRNA-122 plays a critical role in liver homeostasis and hepatocarcinogenesis. J Clin Invest. 122(8), 2884-97(2012).
22. Shu-hao, H. et al. Essential metabolic, anti-inflammatory, and anti-tumorigenic functions of miR-122 in liver. J Clin Invest. 122(8), 2871-2883(2012).
23. Lewis, A. P. & Jopling, C. L. Regulation and biological function of the liver-specific miR-122. Biochem Soc Trans. 38(6), 1553-7(2010).
24. Wang, K. et al. Circulating microRNAs, potential biomarkers for drug-induced liver injury. Proc Natl Acad Sci USA. 106 (11), 4402-4407 (2009).
25. Poy, M. N. et al. A pancreatic islet-specific microRNA regulates insulin secretion. Nature 432, 226-230 (2004).
26. Poy, M. N. et al. miR-375 maintains normal pancreatic α- and β-cell mass. Proc Natl Acad Sci USA 106, 5813-5818 (2009).
27. Ho, T. H. et al. Muscleblind proteins regulate alternative splicing. EMBO J 23, 3103-3112 (2004).
28. Andres, S. F. et al. Insulin receptor isoform switching in intestinal stem cells, progenitors, differentiated lineages and tumors: evidence that IR-B limits proliferation. J Cell Sci 126, 5645-5656 (2013).
29. Suthanthiran, M. et al. Urinary-cell mRNA profile and acute cellular rejection in kidney allografts. N Engl J Med. 369, 20-31 (2013).
30. Li, B. et al. Noninvasive diagnosis of renal-allograft rejection by measurement of messenger RNA for perforin and granzyme B in urine. N Engl J Med. 344, 947-954 (2001).
31. Kotsch, K. et al. Enhanced granulysin mRNA expression in urinary sediment in early and delayed acute renal allograft rejection. Transplantation. 77, 1866-1875 (2004).
32. Tatapudi, R. R. et al. Noninvasive detection of renal allograft inflammation by measurements of mRNA for IP-10 and CXCR3 in urine. Kidney Int. 65, 2390-2397 (2004).
33. Aquino-Dias, E. C. et al. Non-invasive diagnosis of acute rejection in kidney transplants with delayed graft function. Kidney Int. 73, 877-884 (2008).
34. Rood, I. M. et al. Comparison of three methods for isolation of urinary microvesicles to identify biomarkers of nephrotic syndrome. Kidney Int. 78(8), 810-6 (2010).
35. Pisitkun, T. et al. Identification and proteomic profiling of exosomes in human urine. Proc Natl Acad Sci USA. 101(36), 13368-13373 (2004).
36. Gardiner, C. et al. Extracellular vesicle sizing and enumeration by nanoparticle tracking analysis. J Extracell Vesicles. 2, 19671 (2013).
37. Dragovic, R. A. et al. Sizing and phenotyping of cellular vesicles using Nanoparticle Tracking Analysis. Nanomedicine. 7(6), 780-788 (2011).
38. Yusen R. D., et al. The Registry of the International Society for Heart and Lung Transplantation: Thirty-second Official Adult Lung and Heart-Lung Transplantation Report-2015; Focus Theme: Early Graft Failure. J Heart Lung Transplant, 2015. 34 (10): p. 1264-77.
39. Lund L. H., et al. The Registry of the International Society for Heart and Lung Transplantation: Thirty-second Official Adult Heart Transplantation Report—2015; Focus Theme: Early Graft Failure. J Heart Lung Transplant, 2015. 34(10): p. 1244-54.
40. Goldfarb S. B., et al. The Registry of the International Society for Heart and Lung Transplantation: Eighteenth Official Pediatric Lung and Heart-Lung Transplantation Report—2015; Focus Theme: Early Graft Failure. J Heart Lung Transplant, 2015. 34(10): p. 1255-63.
41. Dipchand A. I. et al. International Society for, and T. Lung, The Registry of the International Society for Heart and Lung Transplantation: Eighteenth Official Pediatric Heart Transplantation Report—2015; Focus Theme: Early Graft Failure. J Heart Lung Transplant, 2015. 34(10): p. 1233-43.
42. Josephson M. A., Monitoring and managing graft health in the kidney transplant recipient. Clin J Am Soc Nephrol, 2011. 6(7): p. 1774-80.
43. Johann D. J., Jr. and J. Blonder, Biomarker discovery: tissues versus fluids versus both. Expert Rev Mol Diagn, 2007. 7(5): p. 473-5.
44. Lakkaraju A. and E. Rodriguez-Boulan, Itinerant exosomes: emerging roles in cell and tissue polarity. Trends Cell Biol, 2008. 18(5): p. 199-209.
45. Lo D. J., B. Kaplan, and A. D. Kirk, Biomarkers for kidney transplant rejection. Nat Rev Nephrol, 2014. 10(4): p. 215-25.
46. Hsu S. H., B. Wang, J. Kota, J. Yu, S. Costinean, H. Kutay, L. Yu, S. Bai, K. La Perle, R. R. Chivukula, H. et al. Essential metabolic, anti-inflammatory, and anti-tumorigenic functions of miR-122 in liver. J Clin Invest, 2012. 122(8): p. 2871-83.
47. Li B., C. et al. Noninvasive diagnosis of renal-allograft rejection by measurement of messenger RNA for perforin and granzyme B in urine. N Engl J Med, 2001. 344(13): p. 947-54.
48. Suthanthiran M. et al. Clinical Trials in Organ Transplantation 04 Study, Urinary-cell mRNA profile and acute cellular rejection in kidney allografts. N Engl J Med, 2013. 369(1): p. 20-31.
49. Su D., et al. Angiopoietin-1 production in islets improves islet engraftment and protects islets from cytokine-induced apoptosis. Diabetes, 2007. 56(9): p. 2274-83.
50. Pratt J. R., et al. Local synthesis of complement component C3 regulates acute renal transplant rejection. Nat Med, 2002. 8(6): p. 582-7.
51. Xu M., et al. Expression of hemopexin in acute rejection of rat liver allograft identified by serum proteomic analysis. Shock, 2014. 42(1): p. 65-74.
52. Krikken J. A. et al. High plasma hemopexin activity is an independent risk factor for late graft failure in renal transplant recipients. Transpl Int, 2010. 23(8): p. 805-12.
53. Deng M. C. et al. Investigators, Noninvasive discrimination of rejection in cardiac allograft recipients using gene expression profiling. Am J Transplant, 2006. 6(1): p. 150-60.
54. Gielis E. M. et al. Cell-Free DNA: An Upcoming Biomarker in Transplantation. Am J Transplant, 2015. 15(10): p. 2541-51.
55. Gregson A. L. et al. Altered Exosomal RNA Profiles in Bronchoalveolar Lavage from Lung Transplants with Acute Rejection. Am J Respir Crit Care Med, 2015. 192(12): p. 1490-503.
56. Snyder T. M. et al. Proc Natl Acad Sci USA, 2011. 108(15): p. 6229-34.
57. Garcia Moreira V. et al. Cell-free DNA as a noninvasive acute rejection marker in renal transplantation. Clin Chem, 2009. 55(11): p. 1958-66.
58. Macher H. C., et al., Monitoring of transplanted liver health by quantification of organ-specific genomic marker in circulating DNA from receptor. PLOS One, 2014. 9(12): p. e113987.
59. Sigdel T. K. et al. A rapid noninvasive assay for the detection of renal transplant injury. Transplantation, 2013. 96(1): p. 97-101.
60. Zhang J., et al. Presence of donor- and recipient-derived DNA in cell-free urine samples of renal transplantation recipients: urinary DNA chimerism. Clin Chem, 1999. 45(10): p. 1741-6.
61. Beck J. et al. Digital droplet PCR for rapid quantification of donor DNA in the circulation of transplant recipients as a potential universal biomarker of graft injury. Clin Chem, 2013. 59(12): p. 1732-41.
62. Botezatu I. et al. Genetic analysis of DNA excreted in urine: a new approach for detecting specific genomic DNA sequences from cells dying in an organism. Clin Chem, 2000. 46(8 Pt 1): p. 1078-84.
63. De Vlaminck I. et al. Circulating cell-free DNA enables noninvasive diagnosis of heart transplant rejection. Sci Transl Med, 2014. 6(241): p. 241ra77.
64. Thakur B. K. et al. Double-stranded DNA in exosomes: a novel biomarker in cancer detection. Cell Res, 2014. 24(6): p. 766-9.
65. Zhang L. et al. Microenvironment-induced PTEN loss by exosomal microRNA primes brain metastasis outgrowth. Nature, 2015. 527(7576): p. 100-4.
66. Ho T. H. et al. Muscleblind proteins regulate alternative splicing. EMBO J, 2004. 23(15): p. 3103-12.
67. Pisitkun T., R. F. Shen, and M. A. Knepper, Identification and proteomic profiling of exosomes in human urine. Proc Natl Acad Sci USA, 2004. 101(36): p. 13368-73.
68. Rood I. M. et al. Comparison of three methods for isolation of urinary microvesicles to identify biomarkers of nephrotic syndrome. Kidney Int, 2010. 78(8): p. 810-6.
69. Gardiner C. et al. Extracellular vesicle sizing and enumeration by nanoparticle tracking analysis. J Extracell Vesicles, 2013. 2.

Various publications, patents and patent applications are cited herein, the contents of which are hereby incorporated by reference herein in their entireties.

We claim:

1. A method for enriching donor-derived microvesicles comprising:
(a) obtaining a biological sample from a subject receiving a transplant; and
(b) isolating donor-specific microvesicles from the biological sample, wherein the isolating comprises:
(i) obtaining a fraction of the biological sample by size exclusion chromatography,
(ii) contacting the fraction with a donor-specific anti-HLA antibody, and
(iii) isolating the HLA antibody-bound donor-specific microvesicles in the fraction, thereby obtaining an enriched fraction of donor-specific microvesicles.

2. The method of claim 1, wherein the fraction obtained by size exclusion chromatography comprises recipient-specific microvesicles and the donor-specific microvesicles.

3. The method of claim 1, further comprising contacting the isolated donor-specific microvesicles with an antibody binding a biomarker.

4. The method of claim 3, wherein the biomarker is a protein.

5. The method of claim 4, wherein the protein is a surface protein.

6. The method of claim 3, wherein the biomarker is selected from heat shock cognate protein 71 (Hsc-70), angiopoietin-1, hemopexin, complement C3, ZnT8, GAD65, or a combination thereof.

7. The method of claim 1, wherein the donor-specific microvesicles comprise insulin.

8. The method of claim 1, wherein the subject is a human.

9. The method of claim 1, wherein the transplant is a pancreatic islet transplant, a kidney transplant, a beta cell islet transplant, a pancreatic tissue transplant, or an organ transplant.

10. The method of claim 3, wherein the biomarker is specific to the organ- or tissue-derived microvesicle transplanted to the transplant recipient.

11. The method of claim 3, wherein biomarker is FXYD2.

12. The method of claim 1, wherein the biological sample is a blood or urine sample.

13. The method of claim 1, wherein the donor-specific anti-HLA antibody binds to a major histocompatibility complex protein selected from HLA-A, HLA-B, HLA-C, HLA-DP, HLA-DQ, HLA-DR, or a combination thereof.

14. The method of claim 1, wherein the anti-HLA antibody is conjugated with magnetic beads.


SEQUENCE LISTING (11 sequences)

SEQ ID NO: 1 - DNA, 19 bp, synthetic construct
  misc_feature 1..19: note = Description of Artificial Sequence: Synthetic primer
  source 1..19: mol_type = other DNA
ccttgtgaaccaacacctg

SEQ ID NO: 2 - DNA, 20 bp, synthetic construct
  misc_feature 1..20: note = Description of Artificial Sequence: Synthetic primer
  source 1..20: mol_type = other DNA
gtagaagaagcctcgttccc

SEQ ID NO: 3 - DNA, 22 bp, synthetic construct
  misc_feature 1..22: note = Description of Artificial Sequence: Synthetic primer
  source 1..22: mol_type = other DNA
cccaagattttgtgcagtggtt

SEQ ID NO: 4 - DNA, 24 bp, synthetic construct
  misc_feature 1..24: note = Description of Artificial Sequence: Synthetic primer
  source 1..24: mol_type = other DNA
cagcatgtctctcaaattcatcgt

SEQ ID NO: 5 - DNA, 20 bp, synthetic construct
  misc_feature 1..20: note = Description of Artificial Sequence: Synthetic primer
  source 1..20: mol_type = other DNA
gatgccctggaacctgaaga

SEQ ID NO: 6 - DNA, 22 bp, synthetic construct
  misc_feature 1..22: note = Description of Artificial Sequence: Synthetic primer
  source 1..22: mol_type = other DNA
ccgggtttgagttagcagatct

SEQ ID NO: 7 - DNA, 21 bp, synthetic construct
  misc_feature 1..21: note = Description of Artificial Sequence: Synthetic primer
  source 1..21: mol_type = other DNA
actgggttgtcgatggacggt

SEQ ID NO: 8 - DNA, 21 bp, synthetic construct
  misc_feature 1..21: note = Description of Artificial Sequence: Synthetic primer
  source 1..21: mol_type = other DNA
cggctcatcttcattgatttg

SEQ ID NO: 9 - DNA, 20 bp, synthetic construct
  misc_feature 1..20: note = Description of Artificial Sequence: Synthetic primer
  source 1..20: mol_type = other DNA
ctgtacgccaacacagtgct

SEQ ID NO: 10 - DNA, 20 bp, synthetic construct
  misc_feature 1..20: note = Description of Artificial Sequence: Synthetic primer
  source 1..20: mol_type = other DNA
gctcaggaggagcaatgatc

SEQ ID NO: 11 - DNA, 15 bp, synthetic construct
  misc_feature 1..15: note = Description of Artificial Sequence: Synthetic primer
  source 1..15: mol_type = other DNA
gacaggtggtacctg